(19) United States
(12) Patent Application Publication
(10) Pub. No.: US 2023/0068326 A1
(43) Pub. Date: Mar. 2, 2023

(54) INTRODUCER HAVING CONTROLLABLE OCCLUSION WITH PERFUSION CAPABILITIES

(71) Applicant: RENALPRO MEDICAL, INC., Santa Clara, CA (US)

(72) Inventors: Nicholas C. DeBEER, Montara, CA (US); Daniel J. LEONG, San Mateo, CA (US); James E. TWITCHELL, Redwood City, CA (US)

(21) Appl. No.: 17/760,238

(22) PCT Filed: Mar. 2, 2021
(86) PCT No.: PCT/US21/20550

§ 371 Date: Aug. 5, 2022

Related U.S. Application Data
(60) Provisional application No. 62/984,189, filed on Mar. 2, 2020.

(51) Int. Cl. A61B 17/12; A61M 25/09

(52) U.S. Cl. CPC A61B 17/12036; A61B 17/12109; A61B 17/12145; A61B 17/1215; A61B 17/12045; A61M 25/09; A61B 2017/00477

(57) ABSTRACT

Temporary vascular occlusion devices and methods for use thereof are described which provide temporary vascular occlusion while maintaining distal perfusion along with vascular access. The temporary vascular occlusion device may include a multiple layer scaffold covering having proximal and distal attachment zones separated by an unattached scaffold covering zone where the scaffold covering is adjacent to but not attached directly to the scaffold frame. Devices for a vascular procedure may access the vasculature using a guide catheter in the shaft of the occlusion device. The occlusion device may then be used to provide protection from contrast media used during the vascular procedure conducted using the access provided by the occlusion device.

### CROSS REFERENCE TO RELATED APPLICATIONS

This application claims priority to U.S. Provisional Patent Application No. 62/984,189 filed Mar. 2, 2020, titled “INTRODUCER HAVING CONTROLLABLE OCCLUSION WITH PERFUSION CAPABILITIES,” which is herein incorporated by reference in its entirety.

### INCORPORATION BY REFERENCE

All publications and patent applications mentioned in this specification are herein incorporated by reference to the same extent as if each individual publication or patent application was specifically and individually indicated to be incorporated by reference.

### FIELD

This application relates to various methods and devices for at least partially occluding peripheral blood flow from a blood vessel while maintaining perfusion to blood vessels and structures distal to the occlusion site. Additionally, the occlusion devices may also enable a single vascular access point for simultaneous use of a therapeutic device in conjunction with use of the occlusion device. Still further, embodiments of the present invention relates generally to medical interventions conducted through vessels of the vasculature such as the major arteries, veins and more particularly to access and deployment configurations for conducting percutaneous procedures such as percutaneous valve replacement wherein an introducer sheath combined with a protective device may be utilized to provide minimally-invasive vascular access for passing instruments, prostheses, and other structures along with protection or reduction of harm to exposure to imaging contrast agents used during the above mentioned procedures.

### BACKGROUND

Contrast Induced Acute kidney injury (CI-AKI), also called acute renal failure (ARF), is a rapid loss of kidney function. Its causes are numerous and include low blood volume from any cause, exposure to substances harmful to the kidney, and obstruction of the urinary tract. CI-AKI is diagnosed on the basis of characteristic laboratory findings, such as elevated blood creatinine, or inability of the kidneys to produce sufficient amounts of urine.

Contrast Induced Acute Kidney Injury is diagnosed on the basis of clinical history and laboratory data. A diagnosis is made when there is rapid reduction in kidney function, as measured by serum creatinine, or based on a rapid reduction in urine output, termed oliguria.

For example, the use of intravascular iodinated contrast agents may cause acute kidney injury. In patients receiving intravascular iodine-containing contrast media for angiography, contrast-induced AKI (CI-AKI) is a common problem and is associated with excessive hospitalization cost, morbidity, and mortality. Clinical procedures involving intravascular iodine-containing contrast media injection include for example, percutaneous coronary intervention (PCI), peripheral vascular angiography and intervention, neurological angiography and intervention. Solutions have been suggested for occluding at least partially the blood flow into the renal arteries during procedures where a patient is exposed to intravascular contrast.

Gaining access to the heart and other parts of the cardiovascular anatomy is a continued challenge in cardiovascular medicine. For example, conventional open-surgical procedures for accomplishing tasks such as valve replacement generally involve a thoracotomy and/or creation of one or more access ports across the wall of the heart itself, which is relatively highly invasive and therefore undesirable. Recent progress has been made in the area of catheter-based percutaneous intervention, wherein instrumentation, such as catheters, guidewires, and prostheses, are brought to the heart, brain, or other tissue structures associated with the cardiovascular system through the vessels connected to such structures. These vascular pathways may be quite tortuous and geometrically small, and thus one of the challenges with percutaneous procedures lies in gaining access, conducting the desired interventional and/or diagnostic procedures, and removing the pertinent instrumentation, without damaging the vasculature or associated anatomy.

Conventionally with percutaneous procedures, introducer and dilator sets have been utilized to provide a usable access conduit through an arteriotomy or other surgical access to the vasculature. For procedures on large, relatively straight, and relatively undiseased vessels, such configurations may be adequate, but frequently cardiovascular diagnostic and/or interventional procedures are conducted on diseased cardiovascular systems and in tortuous anatomy. There is a need for better access tools and procedures, which may be utilized to establish vascular access in a relatively efficient geometric package (i.e., in a collapsed state), be expanded in situ as necessary to pass instrumentation, prostheses, or other structures (for example, the un-expanded delivery size of a commercially available aortic valve prosthesis may be up to 18 French or more such as other valves having an un-expanded delivery size of a between 18 and 24 French, depending upon which size is utilized) and to be re-collapsed before or during withdrawal so that the associated anatomy is not undesirably loaded or damaged during such withdrawal. Moreover, the increased availability of such devices and the accompanying use of imaging contrast agents to assist in their proper implantation is leading to an increased risk of patient over exposure to contrast. As such, there remains a need for improvements in introducer sheaths as well as in protection for damage to collateral structures such as the kidneys from exposure to imaging contrast or other agents.

There is also a continuing need for reducing complexity in coordination of device use in vascular procedures. Additionally, it is clinically desirous to reduce where possible the number of access points into the patient's vasculature.

While some solutions have been proposed for vascular occlusion and access, the need for improved methods and especially combination devices remain.

### SUMMARY OF THE DISCLOSURE

In general, in one embodiment, a vascular occlusion device includes a handle having a first part and a second part, an inner shaft coupled to the handle first part, an outer shaft over the inner shaft and coupled to the handle second part, a scaffold structure having a distal end, a scaffold transition zone and a proximal end having one or a plurality of legs wherein the one leg or each leg of the plurality legs is coupled to a distal portion of the inner shaft, wherein the scaffold structure moves from a stowed configuration when the outer shaft is extended over the scaffold structure and a deployed configuration when the outer shaft is retracted from covering the scaffold structure by relative movement of the handle first part and the handle second part; and a scaffold covering over at least a portion of the scaffold structure, the multiple layer scaffold covering having a distal scaffold attachment zone where a portion of the scaffold covering is attached to a distal portion of the scaffold, a proximal scaffold attachment zone where a portion of the scaffold covering is attached to a proximal portion of the scaffold and an unattached zone between the distal attachment zone and the proximal attachment zone wherein the scaffold covering is unattached to an adjacent portion of the scaffold.

This and other embodiments can include one or more of the following features. The plurality of legs can be two legs, three legs or four legs. The scaffold covering can extend from the distal end of the scaffold structure to the one leg or to each of the two legs, three legs or the four legs. The scaffold covering can extend from the distal end of the scaffold structure proximally to cover approximately 20%, 50%, 80% or 100% of the overall length of the scaffold structure. The scaffold covering can extend completely circumferentially about the scaffold structure from the distal attachment zone to the proximal attachment zone. The vascular occlusion device can further include one or more pressure relief features within the scaffold covering. The one or more pressure relief features can be a slit or an opening in the scaffold covering. A distal end portion of the outer sheath can further include an expansion zone. The expansion zone of the outer sheath can include a plurality of sections joined by one or more flexible couplings. Each section of the plurality of sections can include two or three segments. When outer sheath is advanced over the scaffold structure in a deployed configuration, the expansion zone of the outer sheath can transition into a larger diameter to accommodate the scaffold structure of the perfusion device. A distal end portion of the outer sheath can further include an expansion zone having one or a combination of slits, zig-zag cuts, braids or expansion features.

In general, in one embodiment, a combination vascular occlusion and vascular access device includes a handle, an inner shaft coupled to the handle, the inner shaft having a lumen accessible via a hemostasis valve in the handle, an outer shaft over the inner shaft and coupled to the handle, an occlusion with perfusion device having a scaffold structure coupled to the inner shaft, and a scaffold covering over at least a portion of the scaffold structure, the scaffold covering having a distal scaffold attachment zone where a portion of the scaffold covering is attached to a distal portion of the scaffold, a proximal scaffold attachment zone where a portion of the scaffold covering is attached to a proximal portion of the scaffold and an unattached zone between the distal attachment zone and the proximal attachment zone wherein the scaffold covering is unattached to an adjacent portion of the scaffold, and a dilator having an occlusion device pocket proximal to a distal end of the dilator, the occlusion device pocket is sized to hold the occlusion with perfusion device.

This and other embodiments can include one or more of the following features. The occlusion device pocket can be formed by dilator shaft that joins the dilator tip to the dilator body. The length of the occlusion device pocket can be 5 cm, 10 cm, 20 cm or 40 cm. The occlusion device pocket can have a recessed outer diameter of about 0.035 inches or from 0.035 to 0.050 inches and a recessed portion inner diameter of about 0.021 inches or from 0.021 inches to 0.040 inches. The length of the occlusion device pocket can be sufficient to hold an occlusion device having a therapeutic length 1, a therapeutic length 2 or a therapeutic length 3. The scaffold structure having a distal end, a scaffold transition zone and a proximal end having one or a plurality of legs wherein the one leg or each leg of the plurality legs can be coupled to a distal portion of the inner shaft, wherein the scaffold structure can move from a stowed configuration when the outer shaft is extended over the scaffold structure and a deployed configuration when the outer shaft is retracted from covering the scaffold structure. The lumen of the inner shaft sized to allow access for a guide catheter adapted for passing an intravascular device that is one of a diagnostic instrument, or an instrument selected from the group consisting of: an angiography catheter, an intravascular ultrasound testing instrument, or an intravascular optical coherence tomography instrument, and the therapeutic instrument can be preferably a balloon catheter, a drug-eluting balloon catheter, a bare metal stent, a drug-eluting stent, a drug-eluting biodegradable stent, a rotablator, a thrombus suction catheter, a drug administration catheter, a guiding catheter, a support catheter, or a device or a prosthesis delivered as part of a TAVR, TMVR, or TTVR procedure or system. The scaffold covering can extend partially circumferentially about the scaffold structure from the distal attachment zone to the proximal attachment zone with an uncovered scaffold structure. The scaffold covering can extend partially circumferentially about 270 degrees of the scaffold structure from the distal attachment zone to the proximal attachment zone. A first scaffold covering can extend partially circumferentially about 45 degrees of the scaffold structure from the distal attachment zone to the proximal attachment zone and a second scaffold covering extends partially circumferentially about 45 degrees of the scaffold structure from the distal attachment zone to the proximal attachment zone, wherein the first scaffold covering and the second scaffold covering can be on opposite sides of the longitudinal axis of the scaffold structure. The scaffold structure can be formed from slots cut into a tube. Scaffold covering can be formed from multiple layers. The layers of the multiple layer scaffold covering can be selected from ePFTE, PTFE, FEP, polyurethane or silicone. The scaffold covering or the more than one layers of a multiple layer scaffold covering can be applied to a scaffold structure external surface, to a scaffold structure internal surface, to encapsulate the distal scaffold attachment zone and the proximal scaffold attachment zone, as a series of spray coats, dip coats or electron spin coatings to the scaffold structure. The multiple layer scaffold covering can have a thickness of 5-100 microns. The multiple layer scaffold covering can have a thickness of about 0.001 inches in an unattached zone and a thickness of about 0.002 inches in an attached zone.

In general, in one embodiment, a method of providing selective occlusion with distal perfusion using a vascular occlusion device includes advancing the vascular occlusion device in a stowed condition along a blood vessel to a position adjacent to one or more peripheral blood vessels in the portion of the vasculature of the patient selected for occlusion while the vascular occlusion device is tethered to a handle outside of the patient, transitioning the vascular occlusion device from the stowed condition to a deployed condition using the handle wherein the vascular occlusion device at least partially occludes blood flow into the one or more peripheral blood vessels selected for occlusion wherein the position of the vascular occlusion device engages with a superior aspect of the vasculature to direct blood flow into and along a lumen defined by a covered scaffold structure of the vascular occlusion device, deflecting a portion of an unattached zone of the covered scaffold in response to the blood flow through the lumen of the covered scaffold into an adjacent opening of the one or more peripheral blood vessels in the portion of the vasculature of the patient selected for occlusion, transitioning the vascular occlusion device from the deployed condition to the stowed condition using the handle; and withdrawing the vascular occlusion device in the stowed condition from the patient.

This and other embodiments can include one or more of the following features. The one or more peripheral blood vessels in the portion of the vasculature of the patient selected for occlusion can be selected from the group consisting of a hepatic artery, a gastric artery, a celiac trunk, a splenic artery, an adrenal artery, a renal artery, a superior mesenteric artery, an ileocolic artery, a gonadal artery and an inferior mesenteric artery. The covered scaffold unattached zone can further include a position of a portion of the unattached zone to deflect into a portion of at least one of a hepatic artery, a gastric artery, a celiac trunk, a splenic artery, an adrenal artery, a renal artery, a superior mesenteric artery, an ileocolic artery, a gonadal artery and an inferior mesenteric artery when the vascular occlusion device is positioned within a portion of the aorta.

In general, in one embodiment, a method of temporarily occluding a blood vessel, includes advancing a vascular occlusion device in a stowed condition along a blood vessel to a position adjacent to one or more peripheral blood vessels selected for temporary occlusion, transitioning the vascular occlusion device from the stowed condition to a deployed condition wherein the vascular occlusion at least partially occludes blood flow into the one or more peripheral blood vessels selected for temporary occlusion while directing the blood flow through and along a lumen of a covered scaffold of the vascular occlusion device, and transitioning the vascular occlusion device out of the deployed condition to restore blood flow into the one or more peripheral blood vessels selected for temporary occlusion when a period of temporary occlusion is elapsed.

This and other embodiments can include one or more of the following features. Directing the blood flow through and along the lumen of the vascular occlusion device can maintain blood flow to components and vessels distal to the vascular occlusion device while at least partially occluding the blood flow to the one or more peripheral blood vessels. The one or more peripheral blood vessels can be the vasculature of a liver, a kidney, a stomach, a spleen, an intestine, a stomach, an esophagus, or a gonad. The blood vessel can be an aorta and the peripheral blood vessels can be one or more or a combination of: a hepatic artery, a gastric artery, a celiac trunk, a splenic artery, an adrenal artery, a renal artery, a superior mesenteric artery, an ileocolic artery, a gonadal artery and an inferior mesenteric artery.

In general, in one embodiment, a method of providing vascular access and reversibly and temporarily occluding a blood vessel includes advancing an at least partially covered scaffold structure of a tethered vascular occlusion device to a portion of an aorta to be occluded, using a handle of the vascular occlusion device to deploy the at least partially covered scaffold structure within the aorta to occlude partially or completely one or more or a combination of: a hepatic artery, a gastric artery, a celiac trunk, a splenic artery, an adrenal artery, a renal artery, a superior mesenteric artery, an ileocolic artery, a gonadal artery and an inferior mesenteric artery using a portion of a multiple layer scaffold covering while simultaneously allowing perfusion flow through a lumen of the at least partially covered scaffold structure to distal vessels and structures, and using the handle to transition the at least partially covered scaffold structure to a stowed condition between an inner wall of an introducer sheath and an outer wall of a guide catheter within the vascular occlusion device.

This and other embodiments can include one or more of the following features. The insertion of the vascular occlusion device or of the at least partially covered scaffold device to a blood vessel which is the aorta can be introduced by transfemoral artery approach or by trans-brachial artery approach or by trans-radial artery approach. The method can further include advancing the vascular occlusion device over a guidewire into a position adjacent to a landmark of the skeletal anatomy.

In general, in one embodiment, a method of providing vascular occlusion with distal perfusion during an interventional vascular procedure includes accessing an artery of the arterial vasculature with an introducer sheath having an outer wall and an inner wall and a central lumen which is concentric and coaxial with an occlusion with perfusion device in a stowed condition against the introducer sheath inner wall, advancing the introducer sheath with the stowed occlusion with perfusion device into an occlusion position within an aorta with the occlusion with perfusion device adjacent to one or more branch vessels and a distal end of the introducer sheath superior to the one or more branch vessels, withdrawing the introducer sheath to transition the occlusion with perfusion device into a deployed condition within the aorta and in position to reversibly occlude the one or more branch vessels, advancing a guide catheter through a lumen of a shaft of the occlusion with perfusion device, accessing the vasculature with an interventional therapy device via the guide catheter, and performing a catheter based therapy at a vascular access therapy site more than 2 cm distal to the occlusion with perfusion device.

This and other embodiments can include one or more of the following features. The method can further include transitioning the occlusion with perfusion device to a stowed configuration between the inner wall of the introducer sheath and an outer wall of the guide catheter. The method can further include withdrawing a dilator from the lumen of the occlusion with perfusion shaft before performing the step of advancing a guide catheter through a lumen of a shaft of the occlusion with perfusion device. During the step of withdrawing the introducer sheath to transition the occlusion with perfusion device into a deployed condition, the occlusion with perfusion device can move out of contact with an occlusion device pocket of a dilator within the lumen of the shaft of the occlusion with perfusion device. The method can further include transitioning the occlusion with perfusion device to a deployed condition to temporarily and reversibly occlude the one or more branch vessels before performing a step of injecting a contrast solution in support of the catheter based therapy performed using access from the guide catheter in the occlusion with perfusion device. The catheter based therapy site can be at least 8 cm, 10 cm, 20 cm or more distal to the one or more renal ostia. The method can further include transitioning the occlusion with perfusion device from the stowed condition in contact with the outer wall of the guide catheter into a position to at least partially occlude at least one ostia of a renal artery and back to the stowed condition at least once during the step of performing a catheter based therapy. The catheter based therapy site can be at least 8 cm, 10 cm, 20 cm or more distal to the one or more renal ostia. The catheter based therapy site can be at least 8 cm, 10 cm, 20 cm or more distal to the location of the occlusion with perfusion device. The catheter based therapy device can be a prosthetic heart valve or component used as part of a TAVR, TMVR or TTVR procedure or system. The outer diameter of the introducer sheath can be from 7 Fr to 21 Fr. After performing the catheter based therapy, the catheter based therapy device can have a diameter of 15-31 mm. The step of performing a catheter based therapy can further include injecting an amount of contrast agent into the vasculature of the patient. The method can further include transitioning the occlusion with perfusion device from the stowed condition into a position to at least partially occlude at least one ostia of a renal artery for a contrast protection time period and when the contract protection time period has elapsed transitioning the occlusion with perfusion device back to the stowed condition. After the completing of the performing a catheter based therapy and withdrawing all instruments used in the therapy, withdrawing the introducer and occlusion with perfusion device from the artery. The step of transitioning the occlusion with perfusion device between stowed and the position to at least partially occlude one or more ostia of the renal arteries can be performed without adjusting position or introducer or interfering with the working channel used for the distal cardiovascular procedure.

In general, in one embodiment, a vascular occlusion device includes a handle having a slider knob, an inner shaft coupled to the handle, an outer shaft over the inner shaft and coupled within the handle to the slider knob, a scaffold structure having at least two legs and a multiple layer scaffold covering, the at least two legs of the scaffold structure attached to an inner shaft coupler in a distal portion of the inner shaft, and the multiple layer scaffold covering positioned over at least a portion of the scaffold structure. The scaffold structure moves from a stowed condition when the outer shaft is extended over the scaffold structure and a deployed condition when the outer shaft is retracted from covering the scaffold structure.

This and other embodiments can include one or more of the following features. The scaffold structure can be formed from slots cut into a tube. The covering can be applied to nearly all, 80%, 70%, 60%, 50%, 30% or 20% of the scaffold structure. The multiple layer scaffold covering can be made of ePFTE, PTFE, polyurethane, FEP or silicone. The multiple layer scaffold covering can be folded over a proximal portion and a distal portion of the scaffold. After the multiple layer scaffold covering is attached to the scaffold, the scaffold can further include a distal attachment zone, a proximal attachment zone and an unattached zone. The multiple layer scaffold covering can further include a proximal attachment zone, a distal attachment zone and an unattached zone wherein a thickness of the multiple layer covering in the proximal attachment zone and the distal attachment zone is greater than the thickness of the multiple layer scaffold covering in the unattached zone. The multiple layer scaffold covering on the scaffold structure can have a thickness of 5-100 microns. Scaffold structure can have a cylindrical portion and a conical portion wherein the terminal ends of the conical portion are coupled to the inner shaft. The inner shaft can further include one or more spiral cut sections to increase flexibility of the inner shaft. The one or more spiral cut sections can be positioned proximally or distally or both proximal and distal to an inner shaft coupler where the scaffold structure is attached to the inner shaft. The scaffold structure can further include two or more legs. Each of the two or more legs can terminate with a connection tab that is joined to a corresponding key feature on an inner shaft coupler. The multiple layer scaffold covering can include one or more or a pattern of apertures that are shaped, sized or positioned relative to the scaffold structure to modify the amount of distal perfusion provided by the vascular occlusion device in use within the vasculature. The multiple layer scaffold covering can include one or more regular or irregular geometric shapes arranged in a continuous or discontinuous pattern which is selected to adapt the distal perfusion flow profile of the vascular occlusion device in use within the vasculature. When in a stowed configuration within the outer shaft, the overall diameter can be between 0.100 inches and 0.104 inches and when in a deployed configuration the covered scaffold has an outer diameter from 19 to 35 mm. The covered scaffold can have an occlusive length of 40 mm to 100 mm measured from a distal end of the scaffold to a scaffold transition zone. An introducer with an occlusion and perfusion device can be adapted for or used to perform an intravascular procedure in a radial artery, an ulnar artery, a coronary artery, a posterior tibial artery, a fibular artery, an anterior tibial artery, a popliteal artery, a vein, a femoral artery or a portion of an aorta. An introducer with an occlusion and perfusion device can be adapted for or used to perform an intravascular procedure wherein the intravascular device is at least one of a diagnostic instrument, an angiography catheter, a balloon catheter, a drug-eluting balloon catheter, a bare metal stent, a drug-eluting stent, a drug-eluting biodegradable stent, an intravascular ultrasound testing instrument, a rotablator, a thrombus suction catheter, a drug administration catheter, a prosthesis for a portion of the vasculature, a prosthesis for a portion of an organ, a prosthesis for a portion of a heart, a prosthetic heart valve, or a device described in Appendix A or used in TMVR, TTVR, TAVR or other transcatheter coronary repair or replacement component, device, system of procedure. The introducer can further include an expansion capability along all or a portion of the length of the introducer wherein the expansion capability is provided by one or more of a selection of flexible biocompatible polymers alone or in any combination with a braided portion. A portion of an unattached zone of a multiple layer scaffold covering can distend in response to blood flow along a lumen of the scaffold of the vascular occlusion device to occlude an opening of any of a hepatic artery, a gastric artery, a celiac trunk, a splenic artery, an adrenal artery, a renal artery, a superior mesenteric artery, an ileocolic artery, a gonadal artery and an inferior mesenteric artery.

One embodiment is directed to a system for deploying a device to a distal location across a vessel, comprising an elongate introducer sheath and an occlusion with perfusion device stowed within the introducer sheath and proximal to the distal end of the sheath. The elongate introducer sheath tubing may be adapted and configured to expand or be expanded temporarily when capturing a deployed occlusion device, especially when a guide catheter is within the shaft of the occlusion device. Once stowed in this condition, the occlusion device is between an inner wall of the introducer sheath and an outer wall of the guide catheter. Additionally, the expandable section of the outer sheath may be distended when intravascular device is advanced along the lumen or working channel of the introducer.

Upon positioning the introducer into a desired position relative to the renal artery ostia or into a position where the occlusion with perfusion device is positioned to provide partial or substantially complete or complete occlusion of the renal artery ostia to prevent blood flow to the kidneys, the introducer may be configured to be expanded selectively or temporarily into an expanded configuration to facilitate passage of one or more relatively large diameter structures through the lumen of the occlusion with perfusion device that is within the outer sheath. In such a condition, the occlusion with perfusion device may be spaced apart from the outer wall of the introducer to allow for introducer expansion during transit of large diameter devices where the expanded condition of the introducer is beneficially employed. Upon completion of passage of the one or more relatively large diameter structures, the occlusion with perfusion device and/or the outer sheath may be configured to be collapsed back to the collapsed configuration and the occlusion with perfusion device return to the stowed condition to reduce the size of the introducer and occlusion device presented to the blood flow and lumen cross section.

One or both of the introducer or the occlusion with perfusion device may comprise one or more radio-opaque markers coupled to the sheath and configured to assist an operator observing fluoroscopy with positioning of the introducer and occlusion with perfusion device combination relative to the vessel. The introducer may be expandable partially, in sections or substantially expandable by incorporation of one or more of: an open-cell fibrous wall material having a matrix of fibers; a matrix of fibers in a braided pattern; fibers or layers of the introducer comprising a polymeric material selected from one or more of a combination of: polyester, polyamide, polypropylene, and copolymers thereof.

Some portion of the introducer and occlusion with perfusion device may be provided from a substantially non-porous expandable layer may comprise a flexible polymeric material selected from the group consisting of: silicone rubber, olefin block copolymers, and copolymers thereof. Embodiments of the combined introducer and occlusion with perfusion device may be employed to reduce exposure, substantially eliminate exposure or otherwise protect the patient from contrast damage or exposure during intravascular procedures performed using the working channel or lumen of the shaft connected to the occlusion with perfusion device within the introducer. Any of a number of different vascular procedures may be performed by the single point of access provided by the outer sheath and occlusion device combination, such as, for delivery of an implantable prosthesis selected to be passed through the sheath occlusion device combination to a distal location across the vessel or in a vascular procedure where the implantable prosthesis may comprise a cardiac valve prosthesis.

In one aspect provides a device for treating or reduce the risk of acute kidney injury or to provide temporary partial or total occlusion of a blood vessel, comprising: an at least partially covered scaffold on a distal portion of a catheter. The covering or membrane or coating on the scaffold structure provides a functional aspect similar to the disturbing means examples described herein which are associated with a balloon embodiment. In use, the at least partially covered scaffold structure may be positioned to allow some flow, occlude all flow or modulate between flow, no flow or partial flow conditions based on the position of the scaffold structure relative to the blood vessel interior wall.

In another aspect provides a temporary occlusion device for at least partially occluding some or all peripheral vessels from a blood vessel while allowing perfusion to distal vessels and structures. In use when the blood vessel is an aorta, the temporary occlusion device is a partially covered scaffold with an optional position indicator wherein the partially covered scaffold is deployed to occlude completely or partially one or more of a blood vessel in the aorta, the suprarenal aorta or the infrarenal aorta. In another aspect, the at least partially covered scaffold structure is deployed within an aorta to occlude partially or completely one or more or a combination of: a hepatic artery, a gastric artery, a celiac trunk, a splenic artery, an adrenal artery, a renal artery, a superior mesenteric artery, an ileocolic artery, a gonadal artery and an inferior mesenteric artery while simultaneously allowing perfusion flow through or around the at least partially covered scaffold structure to distal vessels and structures.

In some embodiments, the insertion of the at least partially covered scaffold device to an aorta is applied either by transfemoral artery approach or by trans-brachial artery approach or by trans-radial artery approach. In certain embodiments, the catheter further includes an inner shaft adapted for use with a guidewire. In certain embodiments, the method further comprises initially inserting a guidewire into a vessel leading to an aorta.

In general, in one embodiment, a vascular occlusion device includes a handle having a slider, an inner shaft coupled to the handle, an outer shaft over the inner shaft and coupled to the slider, a scaffold structure having a distal end, a scaffold transition zone and a proximal end having a plurality of legs wherein each leg of the plurality legs is coupled to a distal portion of the inner shaft. The scaffold structure moves from a stowed configuration when the outer shaft is extended over the scaffold structure and a deployed configuration when the outer shaft is retracted from covering the scaffold structure. There may be a multiple layer scaffold covering over at least a portion of the scaffold structure. The multiple layer scaffold covering has a distal scaffold attachment zone where a portion of the scaffold covering is attached to a distal portion of the scaffold, a proximal scaffold attachment zone where a portion of the scaffold covering is attached to a proximal portion of the scaffold. There is also an unattached zone between the distal attachment zone and the proximal attachment zone where the scaffold covering is unattached to an adjacent portion of the scaffold.

This and other embodiments include one or more of the following features. The plurality of legs can be two legs or three legs. The scaffold covering can extend from the distal end of the scaffold structure to each of the two legs or the three legs. The scaffold covering can extend from the distal end of the scaffold structure proximally to cover approximately 20%, 50%, 80% or 100% of the overall length of the scaffold structure. The scaffold covering can extend completely circumferentially about the scaffold structure from the distal attachment zone to the proximal attachment zone. The scaffold covering can extend partially circumferentially about the scaffold structure from the distal attachment zone to the proximal attachment zone with an uncovered scaffold structure. The scaffold covering can extend partially circumferentially about 270 degrees of the scaffold structure from the distal attachment zone to the proximal attachment zone. A first scaffold covering can extend partially circumferentially about 45 degrees of the scaffold structure from the distal attachment zone to the proximal attachment zone and a second scaffold covering can extend partially circumferentially about 45 degrees of the scaffold structure from the distal attachment zone to the proximal attachment zone. The first scaffold covering and the second scaffold covering can be on opposite sides of the longitudinal axis of the scaffold structure. The multiple layer scaffold covering can be attached to the scaffold in the distal scaffold attachment zone and in the proximal scaffold attachment zone by encapsulating a portion of the scaffold, by folding over a portion of the multiple layer scaffold covering and encapsulating a portion of the scaffold, by stitching the multiple layer scaffold covering to a portion of the scaffold, or by electrospinning the multiple layer scaffold to a portion of the scaffold. The scaffold structure can be formed from slots cut into a tube. The covering can be applied to nearly all, 80%, 70%, 60%, 50%, 30% or 20% of the scaffold structure. Scaffold covering can be formed from multiple layers. The layers of the multiple layer scaffold covering can be selected from ePFTE, PTFE, FEP, polyurethane or silicone. The scaffold covering or the more than one layers of a multiple layer scaffold covering can be applied to a scaffold structure external surface, to a scaffold structure internal surface, to encapsulate the distal scaffold attachment zone and the proximal scaffold attachment zone, as a series of spray coats, dip coats or electron spin coatings to the scaffold structure. The multiple layer scaffold covering can have a thickness of 5-100 microns. The multiple layer scaffold covering can have a thickness of about 0.001 inches in an unattached zone and a thickness of about 0.002 inches in an attached zone. The vascular occlusion can further include a double gear pinion within the handle that couples the outer shaft to the slider.

In general, in one embodiment, a method of providing selective occlusion with distal perfusion using a vascular occlusion device includes: (1) advancing the vascular occlusion device in a stowed condition along a blood vessel to a position adjacent to one or more peripheral blood vessels in the portion of the vasculature of the patient selected for occlusion while the vascular occlusion device is tethered to a handle outside of the patient; (2) transitioning the vascular occlusion device from the stowed condition to a deployed condition using the handle wherein the vascular occlusion device at least partially occludes blood flow into the one or more peripheral blood vessels selected for occlusion wherein the position of the vascular occlusion device engages with a superior aspect of the vasculature to direct blood flow into and along a lumen defined by a covered scaffold structure of the vascular occlusion device; (3) deflecting a portion of an unattached zone of the covered scaffold in response to the blood flow through the lumen of the covered scaffold into an adjacent opening of the one or more peripheral blood vessels in the portion of the vasculature of the patient selected for occlusion; (4) transitioning the vascular occlusion device from the deployed condition to the stowed condition using the handle; and (5) withdrawing the vascular occlusion device in the stowed condition from the patient.

This and other embodiments can include one or more of the following features. The one or more peripheral blood vessels in the portion of the vasculature of the patient selected for occlusion can be selected from the group consisting of a hepatic artery, a gastric artery, a celiac trunk, a splenic artery, an adrenal artery, a renal artery, a superior mesenteric artery, an ileocolic artery, a gonadal artery and an inferior mesenteric artery. The covered scaffold unattached zone can further include a position of a portion of the unattached zone to deflect into a portion of at least one of a hepatic artery, a gastric artery, a celiac trunk, a splenic artery, an adrenal artery, a renal artery, a superior mesenteric artery, an ileocolic artery, a gonadal artery and an inferior mesenteric artery when the vascular occlusion device is positioned within a portion of the aorta.

In general, in one embodiment, a method of temporarily occluding a blood vessel includes: (1) advancing a vascular occlusion device in a stowed condition along a blood vessel to a position adjacent to one or more peripheral blood vessels selected for temporary occlusion; (2) transitioning the vascular occlusion device from the stowed condition to a deployed condition wherein the vascular occlusion at least partially occludes blood flow into the one or more peripheral blood vessels selected for temporary occlusion while directing the blood flow through and along a lumen of a covered scaffold of the vascular occlusion device; and (3) transitioning the vascular occlusion device out of the deployed condition to restore blood flow into the one or more peripheral blood vessels selected for temporary occlusion when a period of temporary occlusion is elapsed.

This and other embodiments can include one or more of the following features. Directing the blood flow through and along the lumen of the vascular occlusion device can maintain blood flow to components and vessels distal to the vascular occlusion device while at least partially occluding the blood flow to the one or more peripheral blood vessels. The one or more peripheral blood vessels can be the vasculature of a liver, a kidney, a stomach, a spleen, an intestine, a stomach, an esophagus, or a gonad. The blood vessel can be an aorta and the peripheral blood vessels are one or more or a combination of: a hepatic artery, a gastric artery, a celiac trunk, a splenic artery, an adrenal artery, a renal artery, a superior mesenteric artery, an ileocolic artery, a gonadal artery and an inferior mesenteric artery.

In general, in one embodiment, a method of reversibly and temporarily occluding a blood vessel includes: (1) advancing an at least partially covered scaffold structure of a tethered vascular occlusion device to a portion of an aorta to be occluded; and (2) using a handle of the vascular occlusion device to deploy the at least partially covered scaffold structure within the aorta to occlude partially or completely one or more or a combination of: a hepatic artery, a gastric artery, a celiac trunk, a splenic artery, an adrenal artery, a renal artery, a superior mesenteric artery, an ileocolic artery, a gonadal artery and an inferior mesenteric artery using a portion of a multiple layer scaffold covering while simultaneously allowing perfusion flow through a lumen of the at least partially covered scaffold structure to distal vessels and structures.

This and other embodiments can include one or more of the following features. The insertion of the vascular occlusion device or of the at least partially covered scaffold device to a blood vessel which is the aorta can be introduced by transfemoral artery approach or by trans-brachial artery approach or by trans-radial artery approach. The method can further include advancing the vascular occlusion device over a guidewire into a position adjacent to a landmark of the skeletal anatomy. A portion of an unattached zone of a multiple layer scaffold covering can distend in response to blood flow along a lumen of the scaffold of the vascular occlusion device to occlude an opening of any of a hepatic artery, a gastric artery, a celiac trunk, a splenic artery, an adrenal artery, a renal artery, a superior mesenteric artery, an ileocolic artery, a gonadal artery and an inferior mesenteric artery.

In general, in one embodiment, a vascular occlusion device includes a handle having a slider knob, an inner shaft coupled to the handle, an outer shaft over the inner shaft and coupled within the handle to the slider knob, a scaffold structure having at least two legs and a multiple layer scaffold covering, and the multiple layer scaffold covering positioned over at least a portion of the scaffold structure. The at least two legs of the scaffold structure are attached to an inner shaft coupler in a distal portion of the inner shaft. The scaffold structure moves from a stowed condition when the outer shaft is extended over the scaffold structure and a deployed condition when the outer shaft is retracted from covering the scaffold structure.

This and other embodiments can include one or more of the following features. The scaffold structure can be formed from slots cut into a tube. The covering can be applied to nearly all, 80%, 70%, 60%, 50%, 30% or 20% of the scaffold structure. The multiple layer scaffold covering can be made of ePFTE, PTFE, polyurethane, FEP or silicone. The multiple layer scaffold covering can be folded over a proximal portion and a distal portion of the scaffold. After the multiple layer scaffold covering is attached to the scaffold, the scaffold can further include a distal attachment zone, a proximal attachment zone and an unattached zone. The multiple layer scaffold covering can further include a proximal attachment zone, a distal attachment zone and an unattached zone wherein a thickness of the multiple layer covering in the proximal attachment zone and the distal attachment zone is greater than the thickness of the multiple layer scaffold covering in the unattached zone. The multiple layer scaffold covering on the scaffold structure can have a thickness of 5-100 microns. Scaffold structure can have a cylindrical portion and a conical portion. The terminal ends of the conical portion can be coupled to the inner shaft. The inner shaft can further include one or more spiral cut sections to increase flexibility of the inner shaft. The one or more spiral cut sections can be positioned proximally or distally or both proximal and distal to an inner shaft coupler where the scaffold structure is attached to the inner shaft. The scaffold structure can further include two or more legs. Each of the two or more legs can terminate with a connection tab that is joined to a corresponding key feature on an inner shaft coupler. The multiple layer scaffold covering can include one or more or a pattern of apertures that are shaped, sized or positioned relative to the scaffold structure to modify the amount of distal perfusion provided by the vascular occlusion device in use within the vasculature. The multiple layer scaffold covering can include one or more regular or irregular geometric shapes arranged in a continuous or discontinuous pattern which is selected to adapt the distal perfusion flow profile of the vascular occlusion device in use within the vasculature. When in a stowed configuration within the outer shaft the overall diameter can be between 0.100 inches and 0.104 inches and when in a deployed configuration the covered scaffold has an outer diameter from 19 to 35 mm. The covered scaffold can have an occlusive length of 40 mm to 100 mm measured from a distal end of the scaffold to a scaffold transition zone.

### BRIEF DESCRIPTION OF THE DRAWINGS

A better understanding of the features and advantages of the present invention will be obtained by reference to the following detailed description that sets forth illustrative embodiments, in which the principles of the invention are used, and the accompanying drawings of which:

FIG. 1 illustrates a diagram of an exemplary invention device comprises a balloon catheter having a first balloon positioned at the supra-renal aorta position near orifices of bilateral renal arteries for treating acute kidney injury.

FIG. 2 illustrates a diagram of an exemplary invention device for treating acute kidney injury where first balloon is inflated to occlude the orifice of both sides of renal arteries.

FIGS. 3A to 3D are perspective views of first balloon of the invention device. FIG. 3A shows a cylinder-like inflated balloon. FIG. 3C shows the morphology of an exemplary inflated first balloon which is “butter-fly like.” FIG. 3B shows a cross-section view of the cylinder-like inflated balloon of FIG. 3A. FIG. 3D shows a cross-section view of the cylinder-like inflated balloon of FIG. 3B.

FIG. 4 illustrates a diagram showing deflated first balloon 402 and a second balloon 403 is inflated at the location of infra-renal aorta near the orifice of renal arteries.

FIG. 5 illustrates a diagram showing the vortex blood flow caused by 2^{nd} balloon distension.

FIG. 6 shows that a normal saline can be infused from a control box, through the catheter pore 606 into the supra-renal aorta while a second balloon remain inflated.

FIG. 7 shows another aspect of the invention where the first balloon exerts renal artery blood flow augmentation by periodic inflation and deflation of the first balloon.

FIG. 8 shows at the end of PCI, both the first and second balloons are deflated and normal saline as postprocedural hydration continuous infusion.

FIG. 9 shows another aspect of the present invention, where a guidewire is used to guide the device for insertion into the renal artery.

FIG. 10 shows that a spinning propeller is inserted to renal artery and then spins around the central guide wire to augment renal artery blood flow toward the kidney.

FIGS. 11A-11B show variation embodiments of a spinning propeller.

FIGS. 12A-12C shows another embodiment of invention disturbing means where a cone shaped wire reinforced device 1702 partially covered with tunnel membrane 1703 which is deployed from catheter 1701. FIG. 12A shows a side cross section view of an exemplary wire device 1702. FIG. 12B shows the specification of the exemplary wire device 1702 in aorta. FIG. 12C shows that a normal saline or other suitable medicine can be applied via an injection hole (or holes) 1708 via an infusion tube 1707 at the distal opening 1704 or the proximal opening 1705, or combination thereof.

FIGS. 13A-13D illustrate a variation of the embodiment of FIGS. 12A-12C where a cone-cylinder shaped wire device 1802 partially covered with tunnel membrane 1803 is shown. FIG. 13A show a side cross section view of the wire device 1802. FIG. 13B shows a top view of the wire device 1802. FIG. 13C shows a bottom view of the wire device 1802. FIG. 13D provides an isometric view of the wire device 1802.

FIGS. 14A-14C show yet another embodiment of the present disclosure. FIG. 14A shows a catheter shaft comprising an outer shaft, an inner shaft disposed therein. FIG. 14B shows the catheter shaft device with expandable mesh braid coupled to the inner and outer shafts in a low-profile configuration. FIG. 14C shows the catheter shaft device with expandable mesh braid in an expanded configuration.

FIGS. 14D-14G show further embodiments of the present disclosure. FIG. 14D shows a prototype of a catheter shaft device with expandable mesh braid. FIG. 14E shows a fully open mesh braid. FIG. 14F shows a partially collapsed mesh braid. FIG. 14G shows a fully collapsed mesh braid.

FIGS. 15A-15D show the deployment of the embodiment of FIGS. 14A-14G. FIG. 15A shows the insertion of the embodiment into the abdominal aorta. FIG. 15B shows the positioning of the device in the abdominal aorta. FIG. 15C shows the device deployed. FIG. 15D shows the device collapsed.

FIG. 16 is a distal end view of a bare scaffold showing three legs each terminating in a connection tab.

FIG. 17 is an isometric view of the bare scaffold of FIG. 16.

FIG. 18 is a side view of an exemplary scaffold structure having two legs only one visible in this view.

FIG. 19A is a side view of a bare scaffold with two legs for attachment to an inner shaft.

FIG. 19B is an enlarged view of the connection tab on the end of each of the two legs of the scaffold embodiment of FIG. 19A.

FIGS. 20A and 20B are side and perspective views, respectively, of the two key features of an inner shaft coupler that is attached to an inner shaft.

FIG. 20C is an enlarged view of the shaft coupler of FIGS. 21A and 21B showing the detail of a key feature shaped to engage with a connection tab of a scaffold leg.

FIG. 21 is a side view of the two connection tabs of the scaffold legs of the scaffold of FIGS. 19 and 20 engaged with the inner shaft coupler of FIGS. 21A-21C.

FIG. 22 is a perspective view of an occlusion device having a single leg connection to an inner shaft.

FIG. 23A is an exemplary scaffold attached to an inner shaft coupler of an inner shaft having a plurality of spiral cuts.

FIG. 23B is an enlarged view of the scaffold in FIG. 23A showing the spiral cut detail in the distal portion of the inner shaft.

FIG. 24A is an exemplary view of a covered scaffold in a deployed configuration connected to the inner shaft. Openings cut around the legs and the atraumatic tip of the inner shaft are also visible in this view.

FIG. 24B is an enlarged view of the proximal end of the covered scaffold in FIG. 24A showing the covering on the legs extends into the inner shaft coupler. This view also shows the cut outs formed in the covering between the covered legs of the scaffold.

FIG. 25A is a side view of a vascular occlusion device shown without any cover. In this view, the outer shaft is withdrawn using the slider on the handle to position the distal end of the outer shaft at the proximal end of the scaffold. In this embodiment, in the deployed configuration the outer shaft is withdrawn proximal to the scaffold transition zone with the inner shaft coupler remaining within and covered by the outer shaft.

FIG. 25B is a side view of a vascular occlusion device of FIG. 25A. The slider on the handle is in a proximal position to withdraw the outer shaft or sheath from the scaffold allowing the scaffold to transition from a stowed configuration to deployed configuration as illustrated. In this embodiment, in the deployed configuration the outer shaft is withdrawn proximal to the inner shaft coupler.

FIG. 26A is a side view of a vascular occlusion device in a stowed condition with the outer shaft withdrawn slightly to show the stowed distal end of the scaffold as best seen in the enlarged view of FIG. 26B. The slider on the handle is withdrawn slightly from the distal most position on the handle to only slightly withdraw the outer sheath to the illustrated position. Continued proximal movement of the slider will continue to withdraw the outer shaft or sheath from the scaffold allowing the scaffold to transition from a stowed configuration to deployed configuration.

FIG. 26B is an enlarged view of the distal end of the vascular occlusion device in FIG. 26A.

FIG. 27 is an isometric view of a covered scaffold in a deployed configuration. This scaffold embodiment has three legs to be attached to the inner shaft.

FIG. 28A is a side view of a scaffold in a deployed configuration with a transparent covering. This view shows the covering in relation to the scaffold distal end, along the longitudinal length and into the scaffold transition zone where the pattern of a plurality of cells changes to the legs.

FIG. 28B is a view of the covered scaffold in FIG. 28A where the covering is opaque and the scaffold cell pattern is not visible.

FIG. 29A is a side view of a covered scaffold embodiment having two legs for attachment to the central shaft. This covered scaffold embodiment includes proximal and distal scaffold attachment zones and a central covering portion that is unattached to the scaffold. The covering on the legs to the connection tabs and the distal openings are also seen in this view.

FIG. 29B is a perspective view of the proximal end of the covered scaffold of FIG. 29A. The proximal attachment zone is visible in this view through a distal opening.

FIG. 29C is a perspective view of the distal end of the covered scaffold in FIG. 29A. The proximal attachment zone, the distal attachment zone and the distal openings are visible in this view.

FIG. 30 is a side view of an embodiment of a vascular occlusion device in a deployed condition having a 20% scaffold covering. The slider on the handle is in a proximal position to withdraw the outer shaft or sheath from the scaffold allowing the scaffold to transition from a stowed configuration to deployed configuration as illustrated. The 20% scaffold covering distal end aligns with the scaffold distal end and extends proximally along the longitudinal length of the scaffold to cover approximately 20% of the overall length of the scaffold.

FIG. 31 is a side view of an embodiment of a vascular occlusion device in a deployed condition having a 50% scaffold covering. The slider on the handle is in a proximal position to withdraw the outer shaft or sheath from the scaffold allowing the scaffold to transition from a stowed configuration to deployed configuration as illustrated. The 50% scaffold covering distal end aligns proximal to the scaffold distal end and extends proximally along the longitudinal length of the scaffold to cover approximately 50% of the overall length of the scaffold.

FIG. 32 is a side view of an embodiment of a vascular occlusion device in a deployed condition having an 80% scaffold covering. The slider on the handle is in a proximal position to withdraw the outer shaft or sheath from the scaffold allowing the scaffold to transition from a stowed configuration to deployed configuration as illustrated. The 80% scaffold covering distal end aligns with the scaffold distal end and extends proximally along the longitudinal length of the scaffold to cover approximately 80% of the overall length of the scaffold.

FIG. 33A is a side view of an embodiment of a vascular occlusion device in a deployed condition having an 100% scaffold covering. The 100% scaffold covering distal end aligns with the scaffold distal end and extends proximally along the longitudinal length of the scaffold to cover approximately 100% of the overall length of the scaffold with the exception of a small portion of the end of the device as shown. The slider on the handle is in a proximal position to withdraw the outer shaft or sheath from the scaffold allowing the scaffold to transition from a stowed configuration to deployed configuration as illustrated.

FIG. 33B is a side view of an embodiment of a vascular occlusion device in a deployed condition having an 100% scaffold covering similar to FIG. 33A. The slider on the handle is in a proximal position to withdraw the outer shaft or sheath from the scaffold allowing the scaffold to transition from a stowed configuration to deployed configuration as illustrated. This embodiment illustrates a plurality of openings formed in the proximal end of the covering within the scaffold transition zone. The 100% scaffold covering distal end aligns with the scaffold distal end and extends proximally along the longitudinal length of the scaffold to cover approximately 100% of the overall length of the scaffold.

FIG. 34 is a side view of an embodiment of a vascular occlusion device in a deployed condition having a tapered scaffold covering of a partial cylindrical section. The slider on the handle is in a proximal position to withdraw the outer shaft or sheath from the scaffold allowing the scaffold to transition from a stowed configuration to deployed configuration as illustrated. The tapered scaffold covering distal end aligns with the scaffold distal end and extends proximally along the longitudinal length of the scaffold to various distal positions according to the overall covering shape. In this view the exemplary shaped covering extends over only a few cells of the scaffold in the top portion while covering most all of the cells and nearly reaching the scaffold transition zone in the bottom portion.

FIG. 35 is a perspective view of an embodiment of a vascular occlusion device in a deployed configuration having a scaffold covering extending from the distal end of the scaffold to the scaffold transition zone. The slider on the handle is in a proximal position to withdraw the outer shaft or sheath from the scaffold allowing the scaffold to transition from a stowed configuration to deployed configuration as illustrated. A portion of the distal attachment zone is visible in this view along with a section of the spiral cut inner shaft.

FIG. 36 is a perspective view of an embodiment of a vascular occlusion device in a deployed configuration having a scaffold covering extending from the distal end of the scaffold to the scaffold transition zone for about 270 degrees of the scaffold circumference. A portion of the scaffold along the bottom section remains uncovered as shown. The slider on the handle is in a proximal position to withdraw the outer shaft or sheath from the scaffold allowing the scaffold to transition from a stowed configuration to deployed configuration as illustrated. A portion of the distal attachment zone is visible in this view along with a section of the spiral cut inner shaft.

FIG. 37 is a perspective view of an embodiment of a vascular occlusion device in a deployed configuration having a pair of scaffold covering sections extending from the distal end of the scaffold to the scaffold transition zone for about 45 degrees of the scaffold circumference. A portion of the scaffold along the top and bottom section remains uncovered as shown. The slider on the handle is in a proximal position to withdraw the outer shaft or sheath from the scaffold allowing the scaffold to transition from a stowed configuration to deployed configuration as illustrated. A portion of the distal and proximal attachment zones of one of the scaffold covering sections is visible in this view along with a section of the spiral cut inner shaft.

FIG. 38 is a perspective view of an embodiment of a vascular occlusion device in a stowed configuration. The slider on the handle is in a distal position with the outer shaft or sheath over the covered scaffold and maintaining it in a stowed configuration.

FIG. 39A is an enlarged view of the distal end of the stowed vascular occlusion device of FIG. 38.

FIG. 39B is the enlarged view of FIG. 39A showing the proximal movement of the distal end of the outer shaft or sheath as the slider on the handle advances proximally. The distal end of the covered scaffold and a portion of the distal attachment zone is also shown in this view.

FIG. 39C is the view of FIG. 39B showing the result of continued proximal movement of the slider and corresponding proximal movement of the outer shaft allowing more of the covered scaffold to transition into the deployed configuration.

FIG. 40A is a perspective view of an occlusion device having a series of pressure release slits along an upper aspect of the device. The occlusion device is in a deployed configuration with arrows showing blood flow through the device with the release slits closed.

FIG. 40B is a perspective view of the occlusion device of FIG. 40A with an outer sheath moving over the proximal end of the device. The movement of the outer sheath prevents flow out of the proximal end of the device causing blood to urge open and flow through the slits.

FIG. 40C is a perspective view of an occlusion device having a pressure release feature along an upper aspect of the device. The pressure release feature is located under a flexible cover.

FIG. 40D is a perspective view of the occlusion device of FIG. 40C showing the operation of the flexible cover and the pressure release feature. Movement of an outer sheath as shown in FIG. 40B will produce the flow relief mode of FIG. 40D. The flexible cover is shown lifted away from the outer surface of the occlusion device allowing flow through a relief feature in an upper portion of the occlusion device.

FIG. 40E is a perspective view of an occlusion device having a series of pressure release features along an upper aspect of the device. Movement of an outer sheath as in FIG. 40B will produce flow through the relief features.

FIG. 40F is a perspective view of an occlusion device having a pressure release feature provided by the tapered shape of the cover that is wider along the inferior aspect than along the upper aspect. The result is that there more covered scaffold along the bottom portion and less covered scaffold along the upper portion of the device. Movement of an outer sheath as in FIG. 40B will produce flow through the upper portion via the open scaffold portion and around the narrowed cover portion.

FIG. 41A is a perspective view of the vascular occlusion device of FIG. 38 after the slider is moved into the proximal position to fully transition the covered scaffold into the deployed configuration. The slider on the handle is in a proximal position with the outer shaft or sheath withdrawn from the covered scaffold which is shown in a deployed configuration.

FIG. 41B is a perspective view of the vascular occlusion device of FIG. 40 with a section of the outer shaft removed to position the deployed covered scaffold adjacent the handle with the slider shown in the proximal position to fully transition the covered scaffold into the deployed configuration as shown.

FIG. 42 is an exploded view of the handle embodiment of FIG. 41.

FIG. 43 is a cross section view of the handle embodiment of FIG. 41.

FIG. 44A is a cross section of a vascular occlusion device positioned for occlusion of the renal arteries and perfusion of the arterial tree in the lower extremities.

FIG. 44B is an alternative of the embodiment of FIG. 29A-C within a portion of an aorta adjacent to a pair of openings to branch vessels. The cover is attached only at the proximal and distal ends of the device. Portions of the cover that are unattached to the scaffold move in response to blood flow through the device.

FIG. 44C is a view of the device of FIG. 44B showing how unattached portions of the cover have deflected away from the scaffold and at least partially occluded the branch vessels of the aorta.

FIG. 45 is a flow chart of an exemplary method of providing occlusion with perfusion using an embodiment of a vascular occlusion device according to the method 4500.

FIG. 46 is a flow chart of an exemplary method of providing occlusion with perfusion using an embodiment of a vascular occlusion device according to the method 4600.

FIG. 47 is a flow chart of an exemplary method of providing occlusion with perfusion using an embodiment of a vascular occlusion device according to the method 4700.

FIG. 48 is a side view of an exemplary covered scaffold according to one embodiment of the vascular occlusion device. The covered scaffold indicates the distal attachment zone, the proximal attachment zone and the unattached zone that indicate whether a portion of the scaffold covering is joined to the scaffold structure in that zone.

FIG. 49 is a partial exploded view of a portion of each of the individual layers that together form a multiple layer scaffold covering embodiment. Each one of the layers is shown with an arrow indicating an orientation of a characteristic or quality of that layer. Illustrated orientations are provided relative to the central axis of the scaffold structure as parallel (a), transverse (b) or oblique (c) or (d).

FIG. 50 is a plan view of an introducer assembly without the occlusion with perfusion device attached by way of example.

FIG. 51A is a plan view showing a condition in which a diagnostic instrument or a therapeutic instrument is inserted in the introducer sheath occlusion with perfusion device combination with the device in a stowed condition.

FIG. 51B is a plan view showing a condition in which a diagnostic instrument or a therapeutic instrument is inserted in the introducer sheath occlusion with perfusion device combination with the device in a deployed condition.

FIG. 51C is a partial perspective view of an alternative introducer with occlusion device combination in a deployed condition as in FIG. 51B.

FIGS. 52A to 52H are schematic views illustrating, in the order of 52A to 52H, a procedure of percutaneously inserting the introducer sheath into a blood vessel.

FIG. 53 is a schematic view illustrating a condition where the introducer sheath is set indwelling in a blood vessel.

FIGS. 54A to 54C are cross-sectional views, taken in a normal direction relative to an axial direction, illustrating sizes of three kinds of introducer sheaths.

FIG. 55 is a schematic illustration of three different access points where an embodiment of an introducer sheath is inserted into a predetermined blood vessel of a patient for radial access (R), femoral access (F) or lower limb (LL).

FIG. 56 is an exemplary method of using the introducer with an occlusion with perfusion device during an intravascular procedure.

FIG. 57A is a cross section view of an embodiment of a combination access device having an occlusion with perfusion device in a stowed configuration between an inner wall of an outer sheath and a pocket of a modified dilator. The device is shown within an aorta adjacent a pair of branch vessels.

FIG. 57B is a cross section view of FIG. 57A with arrows indicating that the outer sheath is being withdrawn proximally exposing the distal tip of the dilator.

FIG. 57C is a cross section view of FIG. 57B with arrows indicating the continued proximal withdrawal of the outer sheath. A distal portion of the occlusion with perfusion device is transitioned into a deployed configuration and is clear of the distal portion of the dilator pocket.

FIG. 57D is a cross section view of FIG. 57C with arrows indicating the continued proximal withdrawal of the outer sheath to a final position proximal to the scaffold coupling. The occlusion with perfusion device is transitioned into a deployed configuration and is clear of the dilator pocket. An unattached portion of the scaffold covering is shown deflecting into an occluding the branch vessels.

FIG. 57E is a cross section view of FIG. 57D with arrows indicating the proximal withdrawal of the dilator from the occlusion device. The occlusion with perfusion device, outer sheath and guide wire remain in position within the aorta as before.

FIG. 57F is a cross section view of FIG. 57E with arrows indicating the distal advancement of a guide catheter along the guidewire and within the inner shaft and the occlusion device.

FIG. 57G is a cross section view of FIG. 57F with arrows indicating the distal advancement of the outer sheath along occlusion with perfusion device. A proximal portion of the occlusion with perfusion device has transitioned into a stowed condition between an inner wall of the outer sheath and an outer wall of the guide catheter.

FIG. 57H is a cross section view of FIG. 57G with arrows indicating the end of the distal advancement of the outer sheath along occlusion with perfusion device. The occlusion with perfusion device is shown in a stowed condition between an inner wall of the outer sheath and an outer wall of the guide catheter. In this configuration, blood flows along the aorta around the guide catheter and the outer sheath.

FIG. 58 is a cross section view of an alternative embodiment of a combination access device of FIG. 57A having an occlusion with perfusion device in a stowed configuration between an inner wall of an outer sheath and a pocket of a modified dilator. The device is shown within an aorta adjacent a pair of branch vessels. The dilator is modified to form a pocket by using a dilator shaft to couple the dilator tip to the dilator body. The dilator shaft extends proximally into the outer sheath beyond the coupling of the occlusion device scaffold to the inner shaft.

FIG. 59 is a cross section view of an alternative embodiment of a combination access device of FIG. 58 having an occlusion with perfusion device in a stowed configuration between an inner wall of an outer sheath and a pocket of a modified dilator. The device is shown within an aorta adjacent a pair of branch vessels. In this view the dilator is modified to form a pocket by using a dilator shaft to couple the dilator tip to the dilator body. The dilator shaft extends only a small amount into the proximal end of the dilator tip and the distal end of the dilator body.

FIG. 60 is an overall view of an introducer sheath or outer shaft constructed according to an embodiment.

FIG. 61(a) is a side perspective view of an introducer sheath or outer shaft according to an embodiment of the present invention with a portion of an outer elastomeric layer removed.

FIG. 61(b) is an end view section taken from lines 61(b)-61(b) in FIG. 61(a).

FIG. 61(c) is a side perspective view of an introducer sheath or outer shaft according to an embodiment with a portion of an outer elastomeric layer removed.

FIG. 61(d) is an end view section taken from lines 61(d)-61(d) in FIG. 61(c).

FIG. 61(e) is a detail view of an introducer sheath or outer shaft constructed according to an embodiment illustrating a possible configuration with a device disposed in the distal end of the introducer sheath or outer shaft and a clear elastomeric material used as an outer layer.

FIG. 61(f) is a detail view of the tooth configuration taken from circle 61(f) in FIG. 61(e).

FIGS. 62(a), 62(c), 62(e) and 62(g) are detailed views of alternative embodiments of the distal end of an introducer sheath or outershaft.

FIGS. 62(b), 62(d), 62(f) and 62(h) are end views of the detail views of FIGS. 62(a), 62(c), 62(e) and 62(g), respectively with the entire outer elastomeric sleeve removed for clarity.

FIGS. 63(a) and 63(b) illustrate slices or cuts at various orientations.

FIGS. 64(a) and 64(b) are detailed views of an alternative embodiment of the distal end of the introducer or outer sheath using a braid.

FIG. 65A is a perspective view of three sections of an embodiment of an outer sheath expansion zone. Each section includes two segments. Flexible joints or couplings are visible between segments of adjacent sections.

FIG. 65B is an end view of the sheath expansion zone of FIG. 65A taken along section A-A. This view shows a cross section of two segments within a section of the outer sheath expansion zone.

FIG. 65C is an end view of the sheath expansion zone of FIG. 65A taken along section B-B. This view shows a cross section of two segments within a section of the outer sheath expansion zone where a flexible joint attaches within, on or within a portion of a segment.

FIG. 66A is a perspective view of three sections of an embodiment of an outer sheath expansion zone. Each section includes three segments. Flexible joints or couplings are visible between segments of adjacent sections.

FIG. 66B is an end view of the sheath expansion zone of FIG. 66A taken along section A-A. This view shows a cross section of three segments within a section of the outer sheath expansion zone.

FIG. 66C is an end view of the sheath expansion zone of FIG. 66A taken along section B-B. This view shows a cross section of three segments within a section of the outer sheath expansion zone where a flexible joint attaches within, on or within a portion of a segment.

FIG. 67A is a side view of a sheath expansion zone in a non-extended configuration against the outer wall of the inner shaft. The distal end of the distal most section of the expansion zone of the outer shaft is proximal to an occlusion with perfusion device. The occlusion with perfusion device is shown in a deployed configuration.

FIG. 67B is a side view of the sheath expansion zone of FIG. 67A with arrows indicating the distal advancement of the outer sheath. Also shown in this view are arrows indicating the relative displacement of segments and flexible joints in a section that has captured a proximal portion of the deployed occlusion with perfusion device.

FIG. 67C is a side view of the sheath expansion zone of FIG. 67B after the distal advancement of the outer sheath to capture the occlusion with perfusion device. Arrows indicate the relative displacement of segments and flexible joints in the section that have captured the occlusion with perfusion device of FIG. 65A.

FIG. 67D is an end view of the sheath expansion zone of FIG. 67C taken along section A-A where the expansion zone remains against the outer wall of the inner shaft (i.e., unexpanded condition). This view shows a cross section of three segments within a section of the outer sheath expansion zone against the outer wall of the inner shaft.

FIG. 67E is an end view of the sheath expansion zone of FIG. 67C taken along section B-B. This view shows a cross section of three segments within a section of the outer sheath expansion zone that has captured the occlusion with perfusion device. In this configuration the occlusion with perfusion device is in a stowed configuration between the outer wall of the guide catheter (not shown) and the inner wall of the outer sheath expansion zone.

FIG. 68A is a perspective view of a combination occlusion with perfusion device having an outer sheath with an expansion zone. There is an outer sheath handle and inner sheath handle at the proximal end. An occlusion with perfusion device is shown beyond the distal end of the outer sheath in a deployed configuration. A guide catheter is shown within the interior of the deployed occlusion with perfusion device.

FIG. 68B is a side view of a section of the expansion zone of FIG. 68A.

FIG. 68C is a perspective view of the combination occlusion with perfusion device having an outer sheath with an expansion zone of FIG. 68A. Arrows indicate the movement of the outer sheath handle relative to the inner sheath handle to advance the expansion zone along the occlusion with perfusion device. The occlusion with perfusion device is in a stowed condition within the expansion against the outer wall of the guide catheter. The guide catheter is shown extending from and beyond the occlusion device and the distal end of the outer sheath expansion zone.

FIG. 68D is an enlarged view of the final position of the outer sheath handle and inner sheath handle at the proximal end. When the handles are in this position, the occlusion with perfusion device is stowed and distal perfusion occurs while a procedure is performed using the guide catheter or other devices inserted via the lumen of the shaft coupled to the occlusion with perfusion device.

FIG. 69A is a perspective view of a distal end of an additional embodiment of an occlusion with perfusion device in a deployed configuration. The proximal end of the scaffold is attached to the outer shaft. The distal end of the scaffold is attached to the distal end of the inner tube or to an atraumatic tip.

FIG. 69B is a distal end view of the deployed occlusion with perfusion device of FIG. 69A.

FIG. 69C is a perspective view of the occlusion with perfusion device of FIG. 69A transitioned into a stowed configuration by proximal movement of the outer sheath as indicated by the arrow.

FIG. 70 a view of a diagrammatic portion of a torso of a patient. The aorta is illustrated from the aortic arch to the internal and external iliac arteries along with many of the branch vessels. Also visible in this view is a portion of the bony anatomy including the vertebra of the spine, the right and the left pelvic bones, the sacrum and a portion of the coccyx.

FIG. 71 is a table detailing the characteristics and other details of a number of different devices for transcatheter aortic valve replacement (TAVR) procedures.

FIG. 72 is a table detailing the various exemplary sizes of introducers and sheaths used in the delivery of a variety of different sized TAVR devices.

FIG. 73 is a flow chart of an exemplary method of providing occlusion with perfusion using an embodiment of a vascular occlusion device according to the method 7300.

FIG. 74 is a flow chart of an exemplary method of providing occlusion with perfusion using an embodiment of a vascular occlusion device according to the method 7400.

FIG. 75 is a flow chart of an exemplary method of providing occlusion with perfusion using an embodiment of a vascular occlusion device according to the method 7500.

FIG. 76 is a flow chart of an exemplary method of providing occlusion with perfusion using an embodiment of a vascular occlusion device according to the method 7600.

### DETAILED DESCRIPTION

Current treatments/managements for acute kidney injury (AKI), especially contrast-induced acute kidney injury are mainly supportive. They include for example, (1) evaluating and stratifying patients with Mehran risk score before performing percutaneous coronary intervention (PCI), (2) avoiding high-osmolar contrast media by using low-osmolar or iso-osmolar contrast media, (3) reducing the amount of contrast media during PCI, and (4) applying intravenously isotonic sodium chloride solution or sodium bicarbonate solution hours before and after PCI, (5) avoiding use of nephrotoxic drugs (such as nonsteroidal anti-inflammatory drugs, aminoglycosides antibiotics, etc.) See Stevens 1999, Schweiger 2007, Solomon 2010. However, none of them were proven with consistent effect in preventing CI-AKI.

Provided herein are devices and systems that specifically focus on solving the two main pathophysiological culprits of CI-AKI, which are renal outer medulla ischemia and/or prolonged transit of contrast media inside the kidneys.

In some embodiments, there are provided a device for treating acute kidney injury (e.g., CI-AKI) comprising a balloon catheter having at least one balloon, at least one sensor associated with the balloon and a position indication means wherein the balloon occludes the orifice of both sides of renal arteries after inflation while allowing blood flow going through the inflated balloon during application of the device inside abdominal aorta. In some embodiments, the position indication means is a radio-opaque marker, or the like.

Radio opaque markers are vital prerequisites on an increasing number of endovascular medical devices and are appropriately provided on the various embodiments to allow positioning of the temporary occlusion device. The value of radio opaque markers is clearly seen in visibility improvement during deployment of the device. Markers allow for improved tracking and positioning of an implantable device during a procedure using fluoroscopy or radiography.

While some embodiments have been described for use in mitigating CI-AKI, alternative non-balloon based occlusion or partial occlusion devices are also provided. Moreover, such alternative partial or complete peripheral occlusion devices simultaneously provide for distal perfusion blood flow into vessels and structures beyond the occlusion device.

As a result, various occlusion device embodiments may be provided that are adapted and configured to provide temporary occlusion of the peripheral vasculature of the suprarenal and infrarenal abdominal aortic area while maintaining distal perfusion.

Exemplary clinical applications include but are not limited to:

Total or nearly total vascular occlusion of blood flow during the surgical treatment of renal tumors through Retroperitoneoscopic Radical Nephrectomy (RRN), Open Radical Nephrectomy (ORN), Open Nephron-sparing Surgery (ONR), or other surgical interventions where it is beneficial to provide temporary vascular occlusion to peripheral organs.

Temporary vascular occlusion of target organs to prevent the influx of solutions (Contrast Medium, Chemotherapy agents) into sensitive organs.

In some embodiments, there is provided a device for treating acute kidney injury, comprising: a balloon catheter having at least one balloon, at least one sensor associated with the balloon and a position indication means wherein the balloon occlude the orifice of both sides of renal arteries after inflation while allowing blood flow goes through the inflated balloon during application of the device inside abdominal aorta.

The various balloon based device descriptions and associated methods may be modified to accomplish any of the above mentioned or other similar vascular occlusion procedures using an embodiment of a partial covered scaffold occlusion device. Additionally, in some embodiments, there is provided for radial expansion of a nitinol scaffold to allow apposition of the attached membrane to the wall of the aorta, to temporarily occlude the flow of blood to the peripheral vasculature. Importantly, embodiments of the radial occlusion device are designed to allow continued distal perfusion while occluding the entrance into the target arteries. In one embodiment, the catheter based radial occlusion system with simultaneous distal perfusion is advanced over a guidewire. In one aspect, a 0.035″ guidewire is used. In some embodiments, proper position of the occlusion device is obtained using one or more radio opaque marker bands or other suitable structures visible to medical imaging systems.

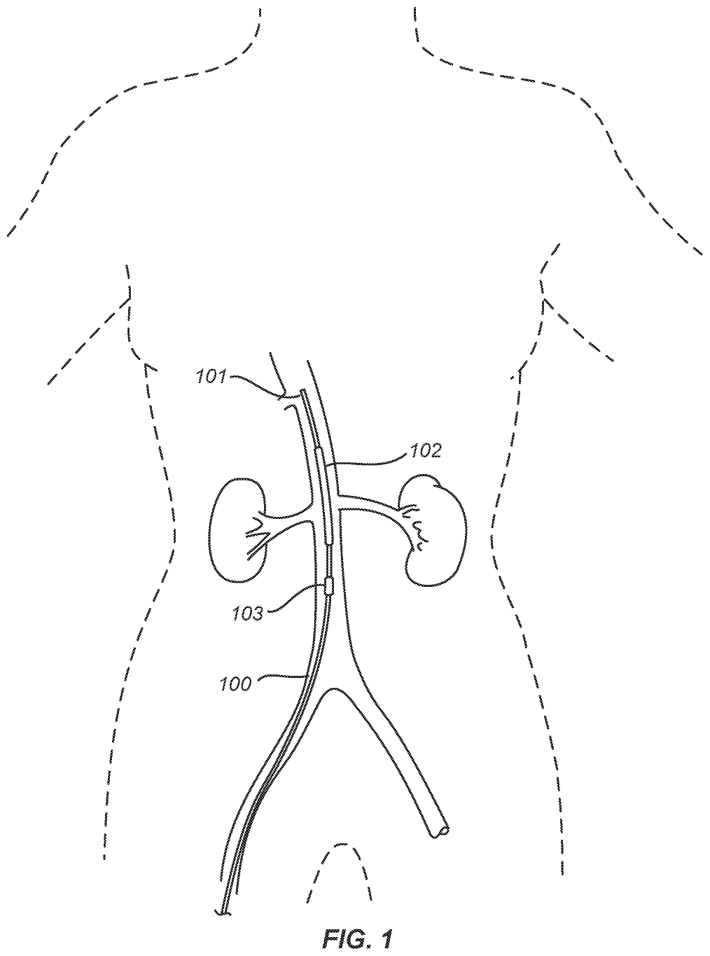

Referring to FIG. 1, an exemplary invention device 100 comprising a balloon catheter 101, a first balloon 102, a second balloon 103 and a radio opaque marker on the tip of the catheter 101 is shown. FIG. 1 shows that the device is inserted via femoral artery and the position of the device is monitored via a radio-opaque marker, or the like. The catheter of the device can be inserted into abdominal aorta by either transfemoral arterial approach or by trans-brachial artery approach or by trans-radial artery approach. The tip with radio-opaque marker is positioned to allow the first balloon at the supra-renal aorta position near orifices of bilateral renal arteries.

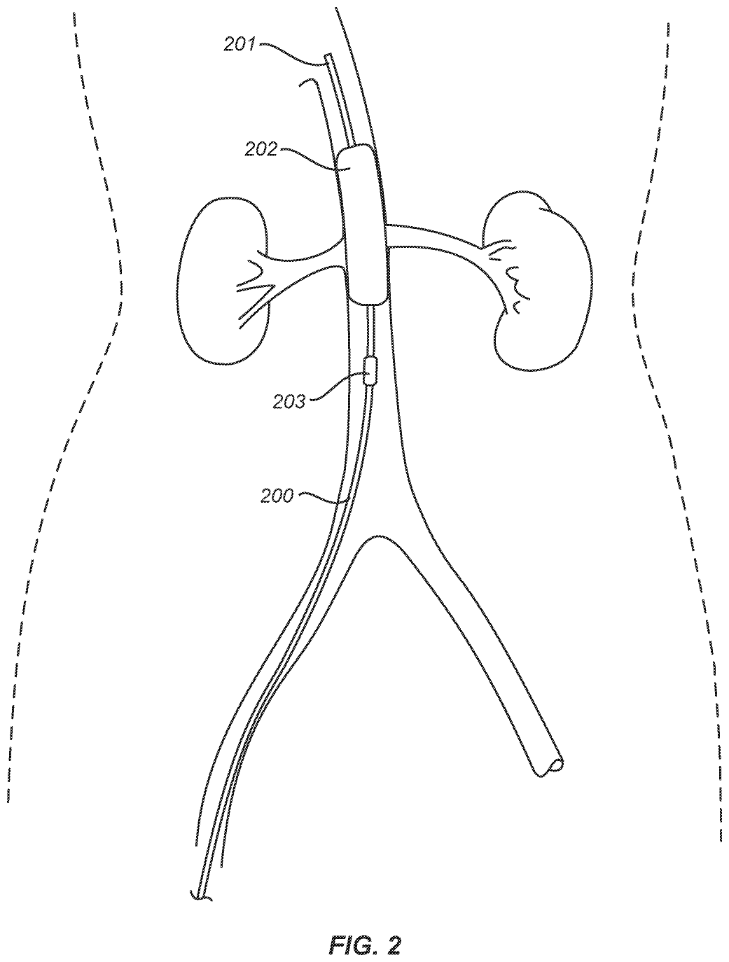

Referring to FIG. 2, a diagram is shown that the device 200 comprising a catheter 201 having a first balloon 202 positioned at the supra-renal aorta position near orifices of bilateral renal arteries and the first balloon 202 is inflated where the inflated first balloon occlude the orifice of both sides of renal arteries so that the bolus influx of contrast media (or any other harmful agents during the application of the invention device) flowing from supra-renal aorta is prevented from entering into renal arteries and cause subsequent toxic effect. The second balloon 203 remains un-inflated.

In certain embodiments, the device comprises a balloon catheter having a first balloon, a second balloon and at least one sensor associated with the second balloon. In some embodiments, the device comprises a balloon catheter having a first balloon, a second balloon and at least one sensor associated with the second balloon.

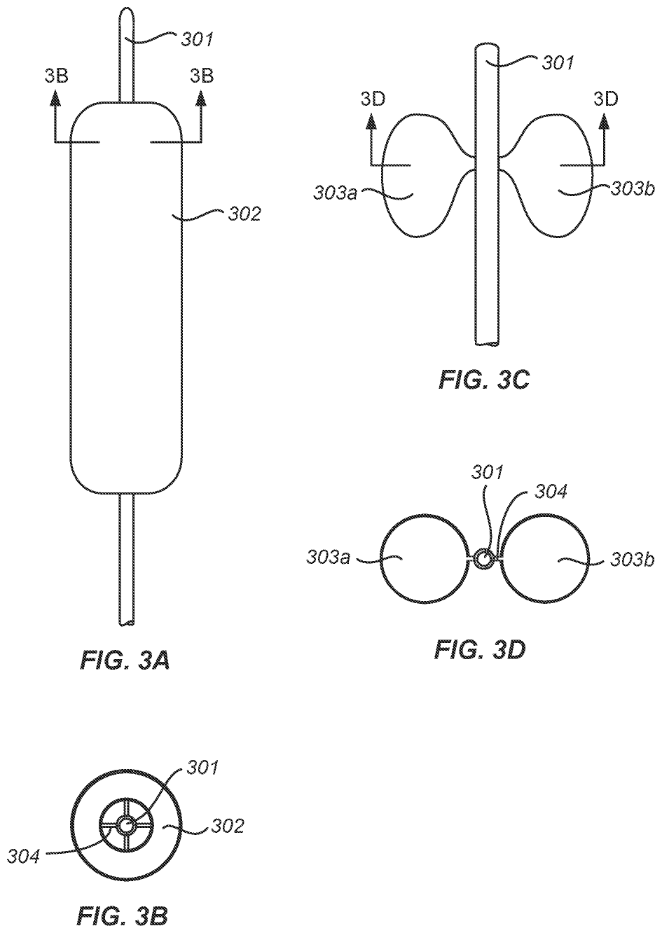

FIGS. 3A to 3D illustrate various embodiments of the first balloon. FIG. 3A shows an inflated first balloon 302 positions along with and circulates the catheter 301. The cross-section view of the inflatable first balloon of FIG. 3A shows a hollow area inside the balloon and outside the catheter 301 (a donut like balloon) allowing blood to flow along the catheter (FIG. 3B). The first balloon 302 is inflated via at least one connection tube 304 from the catheter 301 (four tubes shown in FIG. 3B). FIG. 3C shows other variation of the morphology of inflatable first balloon. A bilateral inflated balloon (303a and 303b) connected to each side of catheter 301 via connection tube 304 to occlude the orifices of both sides of renal arteries are shown in FIG. 3C, which also allows blood to flow along the catheter. FIG. 3D shows the cross-section view of the inflated first balloon of FIG. 3C (a butterfly like balloon). The butterfly like first balloon(s) are connected to the catheter via one or more connection tube 304 (shown one connection tube on each side of the catheter 301). In certain embodiments, the balloon has one, two, three, four or five connection tubes 304 for connection of the first balloon to the catheter and for inflation/deflation means.

In some embodiments, the first balloon is donut-like after inflation. In certain embodiment the first balloon is butterfly-like after inflation.

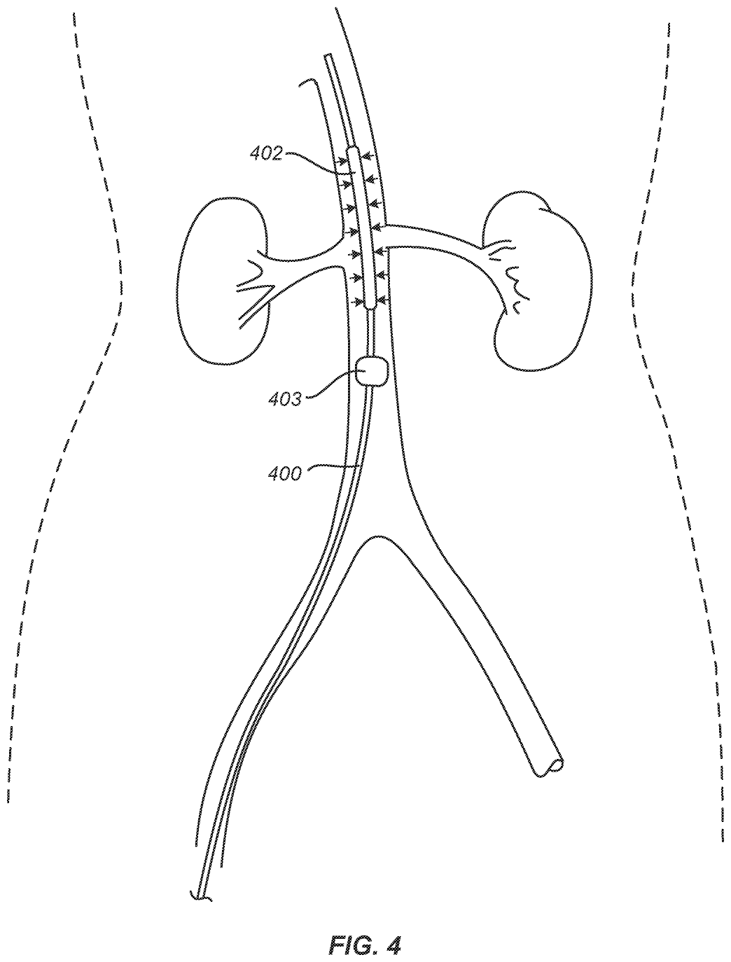

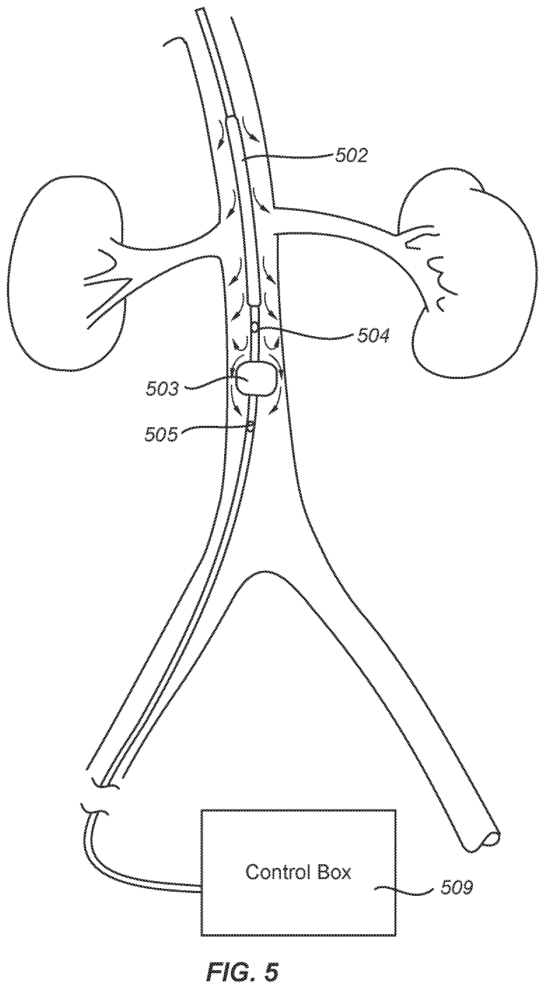

Referring to FIG. 4, it is shown an exemplary device 400 comprising a deflated first balloon 402 after contrast media containing blood passed by and then the second balloon 403 is inflated at the location of infra-renal aorta near the orifice of renal arteries.

The inflation of the second balloon 503 is to the extent not totally occludes the aorta blood flow. As shown in FIG. 5, in the aorta, the vortex blood flow caused by the inflated second balloon distension will facilitate (augment) renal artery blood flow. In some embodiments, there is at least one sensor associated with the first balloon or second balloon for the control of inflation/deflation of either the first and/or second balloon. In some embodiments, the sensor is a pressure sensor. In some embodiments, the sensor is a size measuring sensor related to the size of either the first balloon or the second balloon. As shown in FIG. 5 as a non-limited example, there are one pressure sensor 504 at lower side of the first balloon (or at the upper side of the second balloon) and another pressure sensor 505 at lower side of the second balloon.

The analysis of data from the pressure sensors can be used as instantaneous titration of distention degree of the second balloon to provide adequate pressure gradient, and hence adequate vortex flow into renal arteries. In addition, the altered aorta blood flow will increase the renal artery blood flow, due to the location proximity and the diameter of the distended the second balloon. In some embodiments, the diameter of the distended second balloon is adjustable such that the diameter of the distended balloon is not too large to totally obstruct aorta blood flow and the altered aorta blood flow will not cause inadequacy of aorta blood flow at distal aorta or branches of aorta, i.e. right and left common iliac artery. Furthermore, the aorta wall will not be injured by the balloon distension.

Also shown in FIG. 5, there is a control box 509 outside the patient body, in connection with the balloon catheter. The control box will serve several functions: inflation and deflation of the first and second balloons, pressure sensing and/or measurement of upper and lower pressure sensors, normal saline titration via an included infusion pump with titratable infusion rate.

In some embodiments, there are two sets of pressure sensors, one at the supra-renal aorta side of the balloon, the other at the infra-renal aorta side of the balloon. The two sensors can continuously measure the pressure and the measured data can be exhibited at the control box outside of the patient's body. The pressure difference between the two sensors will be exhibited on the control box. Physicians can read the pressure difference and adjust the size of balloon by way of a control box. Or the control box can do the adjustment of size of balloon automatically.

In some embodiments, the device for treating acute kidney injury further comprises a side aperture on the balloon catheter for application of normal saline or other medication infused from the control box, through the catheter into the supra-renal aorta. In some embodiments, normal saline (or other medication) is applied via a side aperture between the first and second balloon. In some embodiments, normal saline (or other medication) is applied via the tip of catheter.

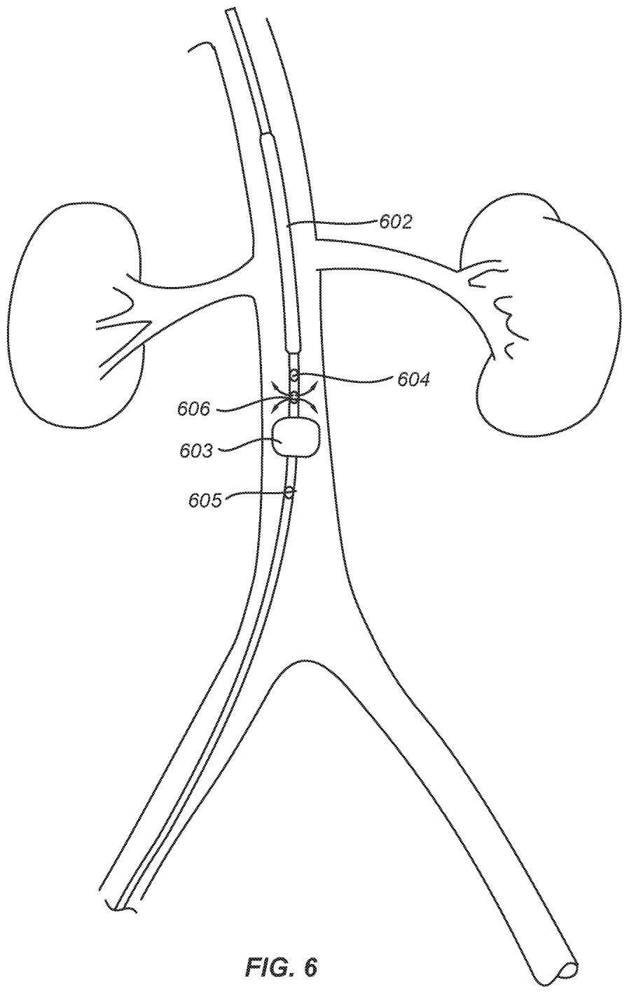

As illustrated in FIG. 6, an exemplary device for treating AKI comprising a first balloon 602, a second balloon 603 (shown inflated), a first sensor 604, a second sensor 605 and a side aperture 606 where normal saline can be infused into the supra-renal aorta via the side aperture 606. By infusion of normal saline into the supra-renal aorta, the renal artery blood flow can be further augmented. Furthermore, it avoids the direct fluid overload burden onto the heart, especially when patients already have congestive heart failure. For the treatment of CI-AKI, the infusion of normal saline into the supra-renal aorta also dilutes the concentration of contrast media in the supra-renal aorta, therefore reduces the concentration of contrast media and thus reduce the adverse effect of hyperviscosity caused by contrast media to the kidneys, after the contrast media flowing into the kidneys. In some embodiments, the infusion rate of normal saline through the side aperture into aorta can be controlled by the control box. In some embodiments, there is a control pump inside the control box to apply normal saline via the side aperture. In some embodiments, the control pump is in a separate unit. In some embodiments, the medication is a vasodilatory agent. In certain embodiments, the vasodilatory agent is Fenoldopam, or the like. In certain embodiments, the medication such as Fenoldopam, or the like is infused via the side aperture for prevention and/or treatment of CI-AKI.

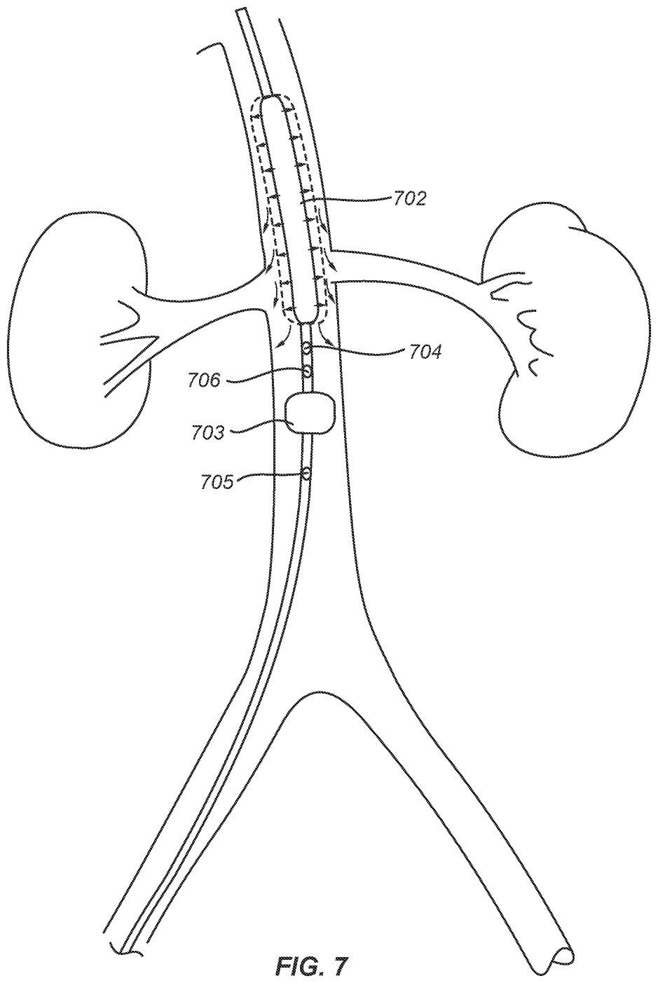

FIG. 7 demonstrates another variation of the invention device comprising a balloon catheter having a first balloon 702, a second balloon 703 (shown inflated), at least one sensor (shown two sensors 704 and 705) and a side aperture where the first balloon 702 can exert renal artery blood flow augmentation by periodic inflation and deflation. As shown in FIG. 7, when the first balloon is inflated, it will not be inflated to totally occlude the orifice of renal arteries as shown in FIG. 2. Such periodic balloon inflation/deflation will cause blood flow into renal arteries.

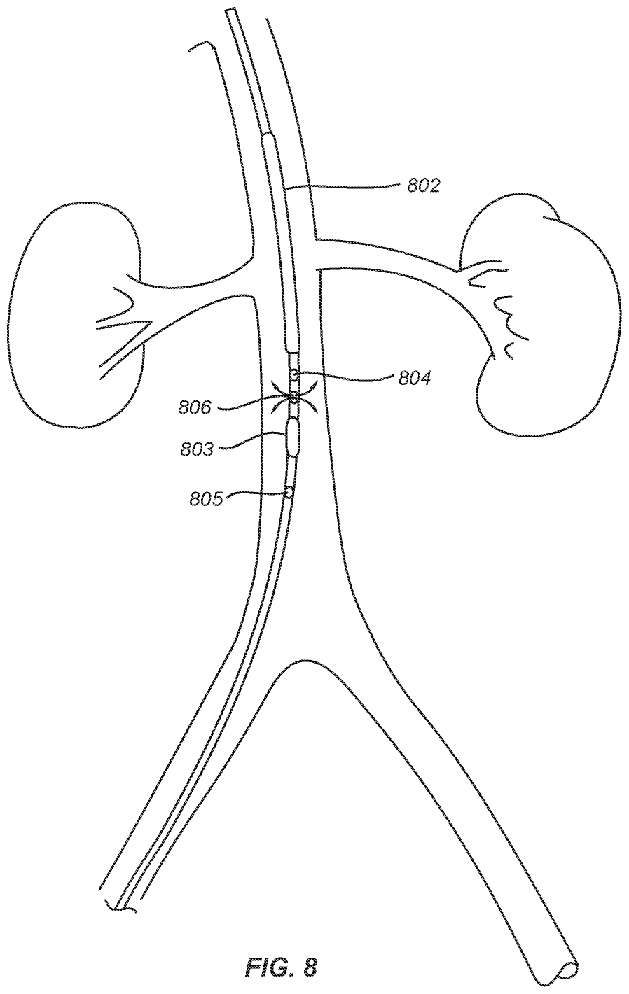

Referring to FIG. 8 at the end of percutaneous coronary intervention (PCI), both the first and second balloons will be deflated and either removed or remained inside aorta and normal saline will be continuously infused via a side aperture 806 as postprocedural hydration.

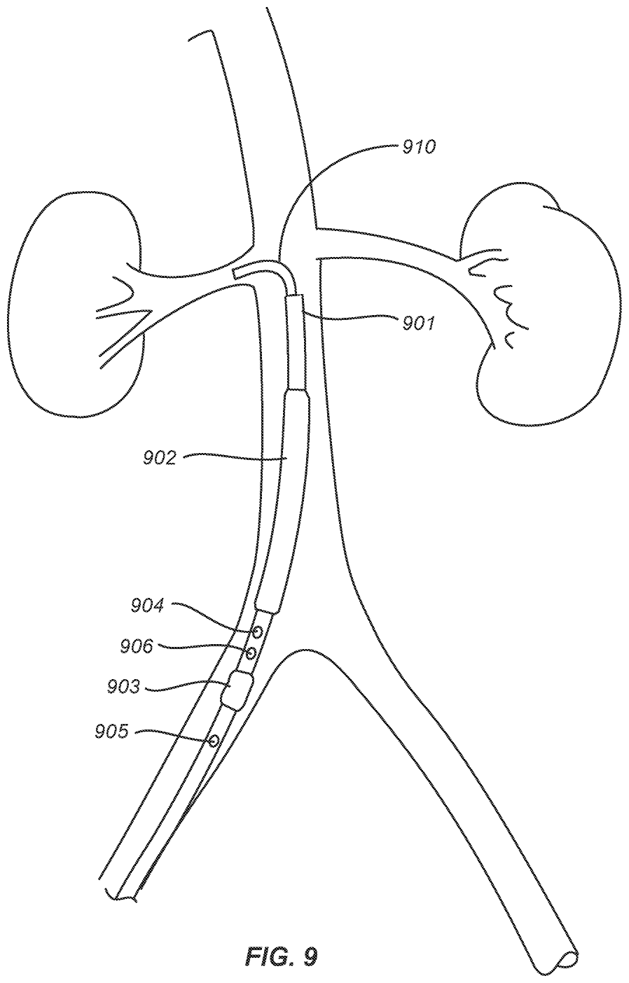

As illustrated in FIG. 9, an exemplary device for treating AKI comprising a catheter 901, a first balloon 902, a second balloon 903, a first sensor 904, a second sensor 905, a side aperture 906 further includes a guidewire 910. The guidewire is inserted into renal artery via a catheter. When guidewire is inside renal artery, the outer sheath catheter is also inserted into renal artery.

FIG. 10 shows that a spinning propeller 1011 is inserted from outer sheath catheter into renal artery through the guidewire 1010. The exemplary unidirectional flow pump such as a spinning propeller then spins around the central guidewire and generate directional augmented renal artery blood flow toward the kidney, hence achieves the goal of augmented renal artery flow.

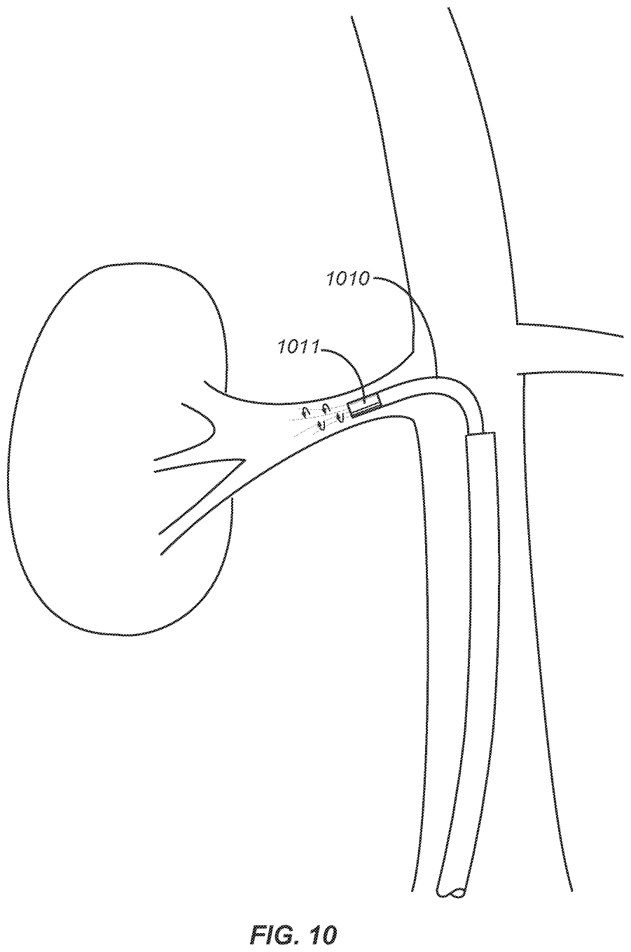

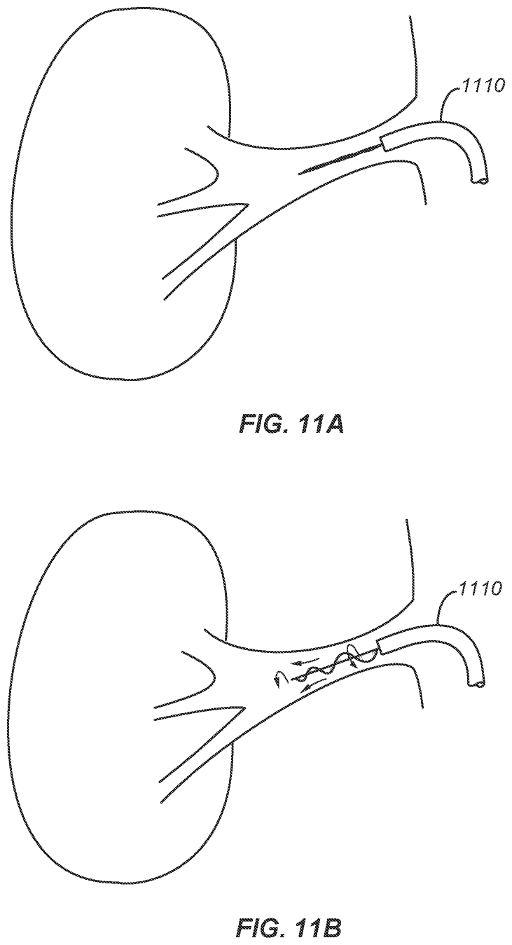

FIGS. 11A and 11B show variations of the spinning propeller. The spinning propeller in some embodiments is wing shape, fin shape, or the like.

In some embodiments, the balloon catheter further includes a guidewire and a spinning propeller. In certain embodiments, the spinning propeller spins around the central guidewire to generate directional augmented renal artery blood flow toward the kidney. In certain embodiments, the spinning propeller is wing shape or fin shape. In certain embodiments, the device further comprises another catheter comprising a guidewire and a spinning propeller to generate directional augmented blood flow to the other kidney. In certain embodiments, the additional catheter having a spinning propeller is functioned independently and simultaneously with the balloon catheter to generate directional augmented blood flow to each side of kidney.

In some embodiments, the infra-renal side of the vascular occlusion device or the disturbing means (such as infra-renal tunnel membrane) can inject saline via injection hole or using the inner shaft into the aorta to dilute the contrast media before it flows into the renal arteries. One or more injection holes may be located along the inner shaft proximal to the atraumatic tip or proximal or distal to the inner shaft coupler 1530.

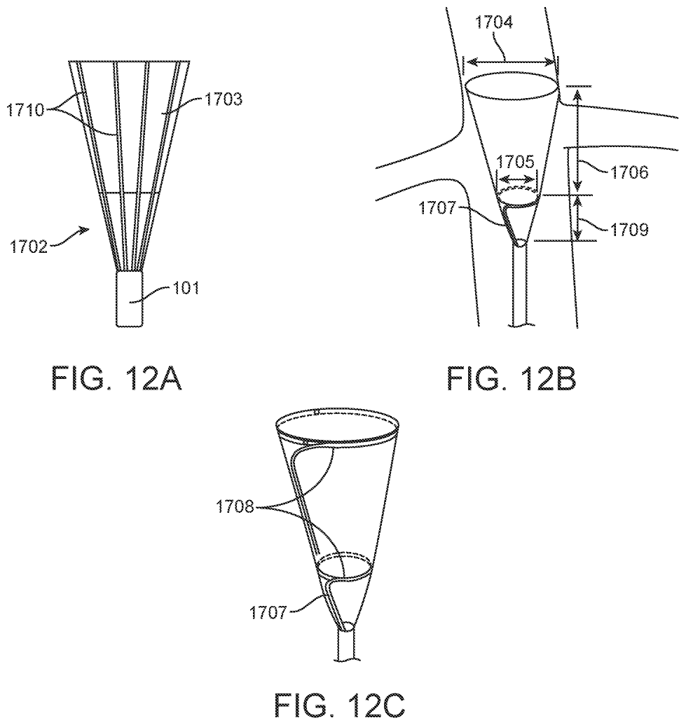

As illustrated in FIG. 12A, which provides yet another embodiment of the flow disturbing means, is a cone shaped wire device 1702 partially covered with tunnel membrane 1703 which is deployed from catheter 1701. FIG. 12B provides an exemplary specification of the cone shaped wire device 1702 of FIG. 12A where the diameter of the distal opening 1704 is about 3 to 3.2 cm or about 3.0 cm. Thus the outer rim of the wire device 1702 is either tightly fitted inside the aorta (of e.g., 3.0 to 3.2 cm diameter) or loosely situated with little space allowing blood seeping through. The diameter of the distal opening 1704 is based on various diameters of an aorta (typically from about 5 cm to about 2 cm) in the patients where the device is deployed. In some embodiments, the distal opening has a diameter of about 5 cm to about 1.5 cm; in some embodiments, the distal opening has a diameter of about 4.5 cm to about 1.7 cm; in some embodiments, the distal opening has a diameter of about 4 cm to about 1.8 cm; about 3.5 cm to about 1.8 cm; or about 3 cm to about 2.0 cm. A tunnel membrane 1703 is covered from the edge of the distal opening 1704 to the proximal opening 1705 of the wire device. The height (1706, see FIG. 12B, where is the distance of blood flowing through) of the tunnel membrane in some embodiments is about 1.5 cm to about 4 cm, about 2 cm to about 3.5 cm, about 2.5 cm to about 3.0 cm (as shown in FIG. 11B is 3 cm). In some embodiments, the height 1706 of the tunnel membrane is about 2 cm, about 3 cm, or about 4 cm. The proximal opening 1705 allows the blood flow through with restricted speed that creates a disturbing of blood flow allowing that the renal arteries intakes blood flow from the infra-renal aorta, where the contrast media has been diluted by the blood flow. To create such an effective blood flow disturbing caused by a disturbing means (e.g, the device 1702), in some embodiments, the diameter of the proximal opening is about one-fourth to about three-fourth of the diameter of the distal opening. In some embodiments, the diameter of the proximal opening is about one-third of the diameter of the distal opening. For example, as shown in FIG. 12B the diameter of the bottom opening 1705 is about 1.0 cm. Relative to where the blood flowing through from the proximal opening, blood releasing height 1709 is designed to be about one-half to about three times of the diameter of the proximal opening. The ratio relationship between blood releasing height 1709 and proximal opening 1705 is based on (1) how the wire device restricts blood flow which creates disturbance, (2) the structural strength of the wire device, and (3) the diameter relationship between the distal opening and the proximal opening.

To support such cone shaped structure, the wire device comprises wires 1710 with at least 3 wires. In some embodiments, there are 4 to 24 wires, 5 to 22 wires, 6 to 20 wires, 8 to 18 wires, or 10 to 16 wires. In some embodiments, there are 3, 4, 5, 6, 7, 8, 9, 10, 11, 12, 13, 14, 15, 16, 17, 18, 19, or 20 wires in the wire device partially covered with tunnel membrane. If needed, a skilled person in the art can prepare a wire device in accordance with the practice of the present invention to any number of wires suitable to provide a disturbing means. The wire may be any superelastic material such as nitinol.

Pseudoelasticity, sometimes called superelasticity, is an elastic (reversible) response to an applied stress, caused by a phase transformation between the austenitic and martensitic phases of a crystal. It is exhibited in shape-memory alloys. Pseudoelasticity is from the reversible motion of domain boundaries during the phase transformation, rather than just bond stretching or the introduction of defects in the crystal lattice (thus it is not true superelasticity but rather pseudoelasticity). Even if the domain boundaries do become pinned, they may be reversed through heating. Thus, a superelastic material may return to its previous shape (hence, shape memory) after the removal of even relatively high applied strains.

The shape memory effect was first observed in AuCd in 1951 and since then it has been observed in numerous other alloy systems. However, only the NiTi alloys and some copper-based alloys have so far been used commercially.

For example, Copper-Zinc-Aluminum (CuZnAl) was the first copper based superelastic material to be commercially exploited and the alloys typically contain 15-30 wt % Zn and 3-7 wt % Al. The Copper-Aluminum, a binary alloy, has a very high transformation temperature and a third element nickel is usually added to produce Copper-Aluminum-Nickel (CuAlNi). Nickel-Titanium Alloys are commercially available as superelastic material such as nitinol. In some embodiments, the superelastic material comprises copper, aluminum, nickel or titanium. In certain embodiments, the superelastic material comprises nickel or titanium, or combination thereof. In certain embodiments, the superelastic material is nitinol.

Specific structures can be formed by routing wires (bending one or a few wires and weaving into final shape) or cutting superelastic tube (laser cutting out the unwanted parts and leaving final wires in place) or cutting superelastic sheet (laser cutting out the unwanted parts and annealing the sheet into a cone shape.

Similarly, in some embodiments, the disturbing means (e.g., the wire device 1702) can inject saline from one or more injection hole 1708 via an infusion tube 1707 at the distal opening 1704 or the proximal opening 1705, or combination thereof into the aorta to dilute the contrast media further before it flows into the renal arteries. See FIG. 12C. In some embodiments, the injection hole(s) is on the catheter, for example at the position close to the tip of the catheter where the disturbing means is deployed.

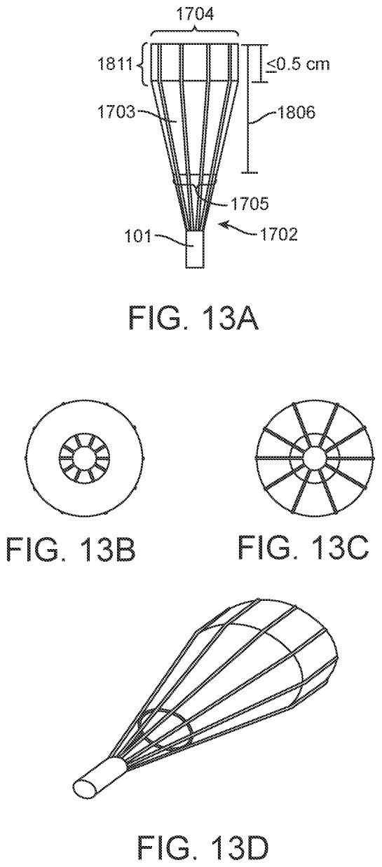

In some embodiments, the cone shaped wire device comprises an upper cylinder portion 1811 as illustrated in FIG. 13A. The upper cylinder portion 1811 is used to form tight contact of the device on the aorta wall. This tight contact supports the device against high pressure due to high blood flow rate. This tight contact prevents contrast media from leaking through the contact interface (without blood seeping through). To avoid occlusion of arteries branching from supra-renal aorta by upper cylinder portion, which is about 0.5 cm apart, the height of the upper cylinder portion should not be more than 0.5 cm to avoid blocking artery branches. The height 1806 of the distal opening to the proximal opening should be about 1.5 cm to about 4 cm, about 2 cm to about 3.5 cm, or about 2.5 cm to about 3.0 cm.

As illustrated in FIG. 13A (a side view), which provides yet a variation of the embodiment of FIGS. 12A-12C, a cone-cylinder shaped wire device 1802 partially covered with a coating, sheet or tunnel membrane 1803 from the rim of the distal opening 1804 to proximal opening of 1805, which is deployed from catheter 1801. FIG. 13B shows a top view of the wire device 1802. FIG. 13C shows a bottom view of the wire device 1802. FIG. 13D provide an isometric view of the wire device 1802.

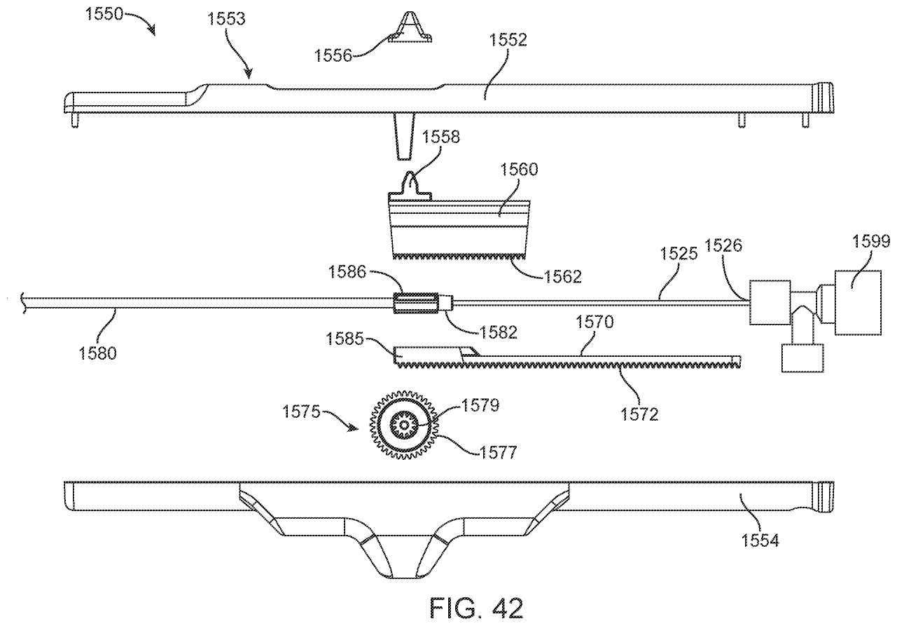

In yet another embodiment, first and second balloons 102, 103 may be replaced by an expanded foam or other biocompatible sealant structure that may be compressed against the vessel wall. The deployed sealant structure under radial force generated by the wire structure or other scaffold embodiment seals against the vessel wall sufficient to fully or at least substantially seal to the vessel wall such that all or substantially all of the blood flow within the vessel flows through the tunnel membrane. Additionally or optionally, the tunnel membrane may be solid or include apertures to allow for various amounts of localized perfusion (see for example FIGS. 42-47). In yet another aspect, balloons 102 and 103 are replaced by a sleeve. The sleeve may be formed from an ePTFE or other compressible biocompatible material. In yet another aspect, the proximal and distal structures about the tunnel membrane may be coated wires, or a hydrogel. In still further alternative structures, one or more of the wires 107 may extend to the ends of the structure, or optionally include a zig-zag pattern and formed from nitinol for self-expansion. It is to be appreciated that in some embodiments, no balloons are utilized but the amount of sealing for a particular embodiment is provided by an alternative radial force sealing structure as describe herein.

The position indication means 105 may for example be a radio-opaque marker. One or more position indication means 105 may be located on the tip of the catheter 101, on the proximal balloon 103, on the distal balloon 102, or any combination thereof. The position indication means 105 may be used to monitor the position of the device 100 upon insertion, during use, and during removal. The device 100 may be inserted into the abdominal aorta for example by using either a trans-femoral arterial approach, a trans-brachial artery approach, or a trans-radial artery approach.

In some embodiments, the aperture 106 and the surrounding wire 107 comprise at least one set of the aperture 106 and the surrounding wire 107 on the tunnel membrane. In some embodiments, there are one to four sets, two to six sets, three to nine sets, four to twelve sets, five to fifteen sets, or six to eighteen sets. In some embodiments, there may be 1, 2, 3, 4, 5, 6, 7, 8, 9, 10, 11, 12, 13, 14, 15, 16, 17, or 18 sets of the aperture and the surrounding wire on the tunnel membrane. If needed, a person skilled in the art can prepare a wire device in accordance with the practice of the present disclosure to any number sets of the aperture and the surrounding wire suitable to provide a flow passage means. The wire may be any superelastic material, for example nitinol. The wire may be made of any superelastic or pseudoelastic material, for example nitinol, alloys of nickel-titanium, or any combination thereof. In some embodiments, the superelastic material may comprise one or more of nickel, titanium, or any combination thereof. Alternatively, any of the above may be modified for use as a wire frame scaffold used with a covering, membrane, coating or tunnel membrane described herein without provision for an aperture 106. Additionally or optionally, the braid embodiments described herein may be include interleaved longitudinal wires to provide an adjustable stiffness. Additionally, the longitudinal wires are provided so as to remain aligned to the central axis of the catheter. Still further, aspects of the fabrication technique and weave patterns used in the braid structure are utilized to modify or adjust a foreshortening characteristic of the braid structure when used as an partially covered scaffold vascular occlusion device.

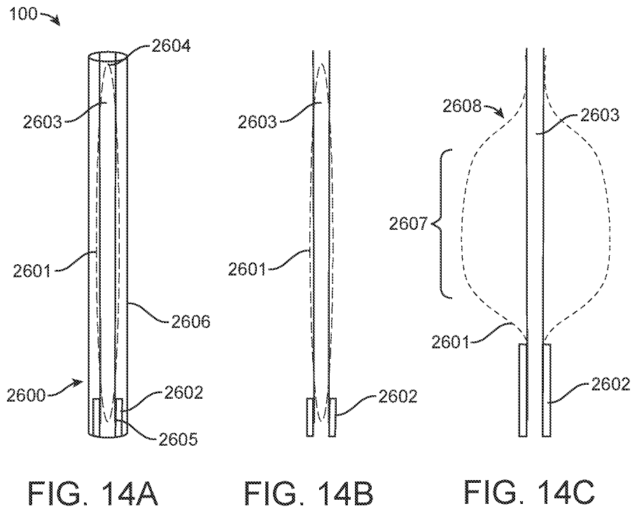

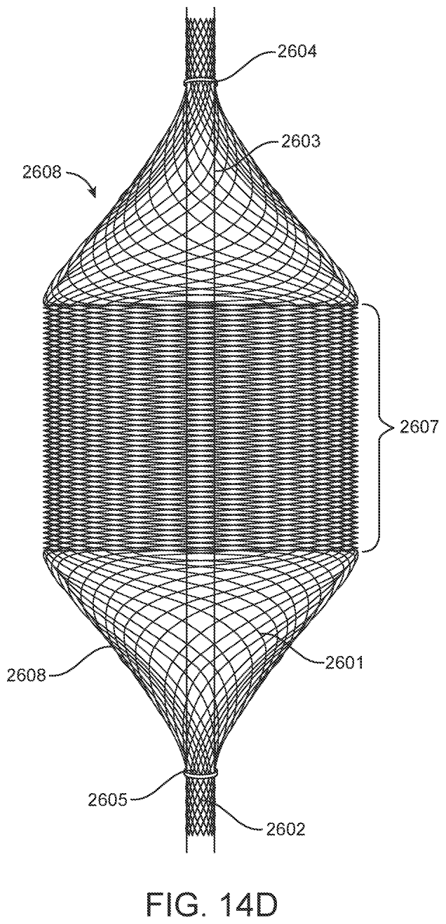

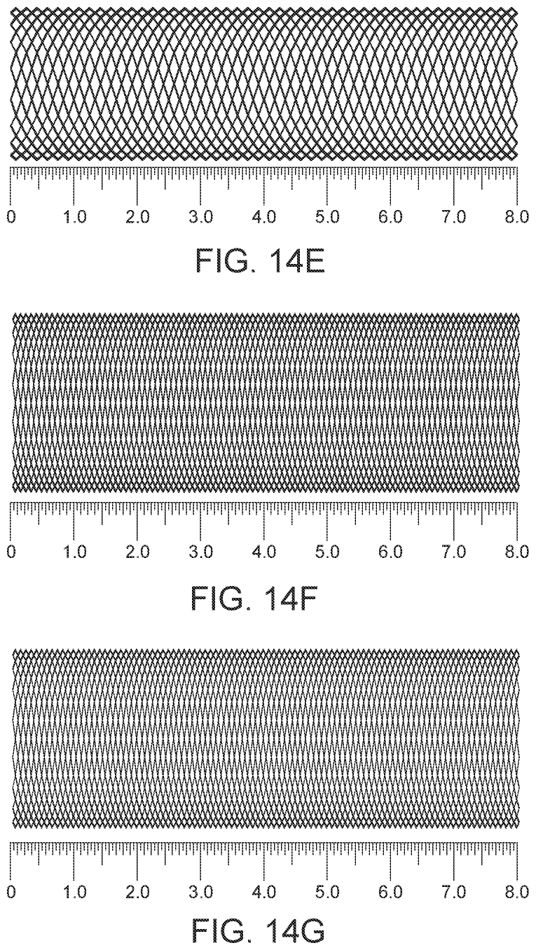

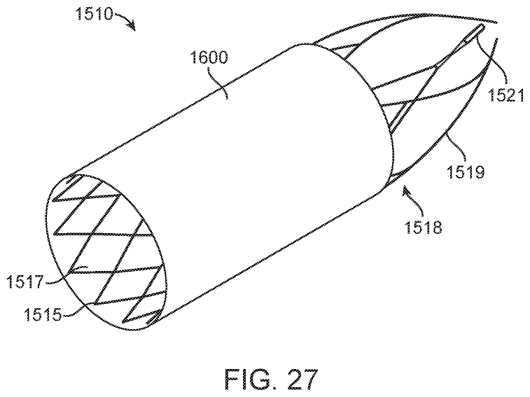

FIGS. 14A-14G show yet another embodiment of the present disclosure. The catheter device 100 may comprise a catheter shaft 2600 actuated to deploy an occlusive element 2601 to occlude the renal artery openings. The occlusive element 2601 may, for example, be an expandable mesh braid. In additional embodiments, the mesh braid is at least partially covered by a covering, membrane, coating or tunnel membrane described to enhance the ability to provide complete or partial occlusion with distal profusion. The covering is omitted from the various views so as not to obscure details of the braid structures. The cover, coating, membrane or tunnel membrane may be a full covering of the underlying structure or scaffold including a partial, single or multiple layer scaffold covering implemented as shown in FIGS. 27, 28B, 29A-29C, 30, 31, 32, 33A, 33B, 34, 35, 36, 37, 39C, 40 and 41. In other aspects where the scaffold is formed from an expandable mesh braid, this structure may comprise a tubular, metal mesh braid comprising a plurality of mesh filaments. The expandable mesh braid may comprise a shape-memory material such as Nitinol and may be biased to be in the expanded configuration. The device may further comprise a position indication features, for example, at least a portion of the catheter device may be radio-opaque. In one aspect, the atraumatic tip 1532 of the inner shaft 1525 is radio-opaque.

The expandable mesh braid or the scaffold may for example be made of a superelastic material such as nitinol. The braid or scaffold may be made of any superelastic or pseudoelastic material, for example nitinol, alloys of nickel-titanium, or any combination thereof. In some embodiments, the superelastic material may comprise one or more of copper, aluminum, nickel, titanium, or any combination thereof. The expandable mesh braid may for example be made of steel or any other mesh-grade material. The expandable mesh braid may be provided with a tunnel or occlusion membrane 1600 embodiment as described herein. Optionally, the braid or scaffold or portion thereof may be coated such as with a hydrophobic coating, a hydrophilic coating, or a tacky coating for enhanced occlusion properties. Additionally or optionally, one or both of the inner and outer braid surfaces may be coated with ePTFE, PTFE, polyurethane or silicone. In some embodiments, the thickness of the coating is from 5 to 100 microns. Still further, the shape of the braid or scaffold may be adjusted to better fit into the geometry of the abdominal aorta, for example the diameter of the lower part of the braid may be smaller than the diameter of the upper part of the braid. It is to be appreciated that these coating concepts may also be applied to the various scaffold embodiments described herein.

FIG. 14A shows a catheter shaft 2600 comprising an outer shaft 2602 and an inner shaft 2603 disposed therein which are translatable relative to one another. The distal end 2604 of the expandable mesh braid 2601 may be coupled to the inner shaft 2603 while the proximal end 2605 of the expandable mesh braid 2601 may be coupled to the outer shaft 2602 such that translation of the inner shaft 2603 relative to the outer shaft 2602 deploys or collapses the expandable mesh braid 2601. The catheter shaft 2600 may further comprise a cover 2606 to protect the catheter shaft device 100 during insertion into the abdominal aorta. The cover 2606 may be removed upon positioning the catheter shaft device 2600 at a desired location.

FIG. 14B shows the catheter shaft device 100 with expandable mesh braid 2601 coupled to the inner 2603 and outer 2602 shafts. The expandable mesh braid 2601 is shown in a low-profile configuration which may be used for delivery of the device 100 through the vasculature prior to deployment. The low-profile configuration may be axially elongated and radially collapsed.

FIG. 14C shows the catheter shaft device 100 following actuation of the inner shaft 2603 relative to the outer shaft 2602 for deployment of the expandable mesh braid 2601. The expandable mesh braid 2601 is shown in an expanded configuration such that the device 100 occludes the renal artery ostia (also referred to herein as orifices) to prevent contrast agent from flowing into the renal arteries of a patient when a bolus of the contrast agent has been introduced into the vasculature. The expanded configuration may be axially foreshortened and radially expanded. In the expanded configuration, the expandable mesh braid 2601 may comprise a minimally porous portion 2607, for example a high-density mesh brain filament portion. The minimally porous portion 2607 may be a region where the braid 2601 is axially foreshortened to increase filament density. The expandable mesh braid 2601 in the expanded configuration may comprise one or more porous end portions 2608 adjacent to the minimally porous portion 2607 so as to allow blood to flow through the braid 2601 from the supra-renal aorta to the infra-renal aorta, bypassing the occluded renal arteries. The one or more porous end portions 2607 may comprise low mesh braid filament density portions.

Actuation of the catheter shaft for deployment of the expandable mesh braid may, for example, comprise translating the inner and outer shafts such that the distal end of the outer shaft moves closer to the distal end of the inner shaft.

FIG. 14D shows a prototype of a catheter shaft device 2600 with expandable mesh braid 2601. The embodiment comprises a tubular metal mesh braid 2601 comprising a plurality of mesh filaments made of Nitinol, an outer shaft 2602, and an inner shaft 2603. The distal end 2604 of the expandable mesh braid 2601 is coupled to the inner shaft 2603 while the proximal end 2605 of the expandable mesh braid 2601 is coupled to the outer shaft 2602. Translation of the inner shaft 2603 relative to the outer shaft 2602 deploys or collapses the expandable mesh braid along with any attached coating, covering, or membrane. In its expanded configuration, the expandable mesh braid 2601 comprises a minimally porous portion 2607 with which to occlude the orifices of the renal arteries. The expandable mesh braid further comprises two porous end portions 2608 which may allow blood to flow through the braid 2601 from the supra-renal aorta to the infra-renal aorta, bypassing the occluded renal arteries. FIG. 14E shows the expandable mesh braid 2601 with fully open mesh. FIG. 14F shows the expandable mesh braid 2601 with a partially collapsed mesh. FIG. 14G shows the expandable mesh braid 2601 with fully collapsed mesh.

The vascular occlusion device 1500 may further comprise a time-delayed release mechanism configured to automatically collapse the expandable occlusion structure (ie., mesh braid or scaffold) after a pre-determined amount of time following deployment. The time-delayed release mechanism may, for example, comprise an energy accumulation and storage component and a time-delay component. For example, the time-delayed release mechanism may comprise a spring with a frictional damper, an example of which may be included in the handle 1550. The energy accumulation and storage component may for example be a spring or spring-coil or the like. The time-delayed release mechanism may for example be adjustable by one or more of the user, the manufacturer, or both. The time-delayed release mechanism may further comprise a synchronization component to synchronize the injection of a contrast media or other harmful agent with the transition of the vascular occlusion device between a stowed configuration and a deployed configuration to aid in preventing harm to structures vascularized by the peripheral vessels that are subject to selective occlusion by operation of the device. For example, injection of contract may be synchronized with occlusion of the renal arteries by the expandable mesh braid or covered scaffold such that a contrast media may be prevented or substantially prevented or greatly reduced amounts from entering the renal arteries.

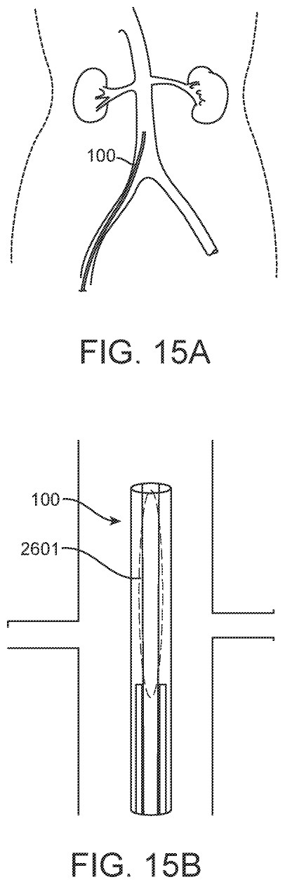

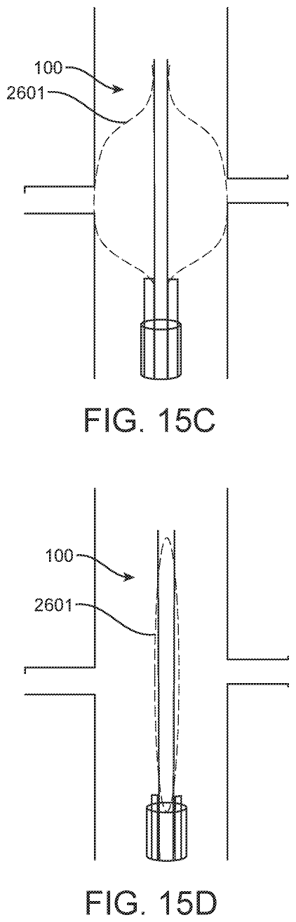

FIGS. 15A-15D show the deployment of the embodiment of FIGS. 14A-14G. Similar deployment steps may be used for all of the embodiments described herein. As shown in FIG. 15A, the device 100 may be inserted into the abdominal aorta via the femoral artery. Alternatively, the device 100 may be inserted into the abdominal aorta via the branchial or radial arteries. As shown in FIG. 15B, the device 100 may be guided to a desired location within the abdominal aorta by monitoring a position indication means, for example a radio-opaque marker or a radio-opaque portion of the catheter. The device 100 may for example be positioned such that deployment of the expandable mesh braid 2601 occludes the orifices of the renal arteries. FIG. 15C shows the expandable mesh braid 2601 deployed at a desired position so as to occlude the orifices of the renal arteries. The expandable mesh braid 2601 may be deployed prior to or simultaneously with injection of a contrast agent into the abdominal aorta of a patient so as to prevent the contrast agent from entering the renal arteries. After the bolus of contrast agent has been introduced, the expandable mesh braid 2601 may be collapsed to allow blood flow to the renal arteries to resume, as shown in FIG. 15D.

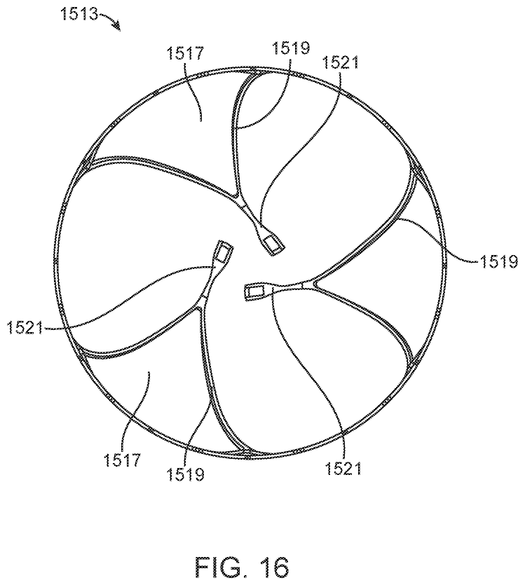

Various embodiments of a vascular occlusion device 1500 are described and illustrated herein and with specific reference to FIGS. 16-49. In general, these embodiments along with those detailed in FIGS. 1-15, relate to a vascular occlusion device configured with structure (e.g., a scaffold structure in relation to FIGS. 16-49) that is adapted to provide selective occlusion with perfusion when appropriately positioned within the vasculature. An exemplary vascular occlusion device 1500 includes a handle 1550, an outer shaft 1580, an inner shaft or hypotube 1525 and a covered scaffold coupled to the distal end of the inner shaft 1525. A slider 1556 on the handle 1550 is coupled to the outer shaft 1580. As the slider 1556 moves along a slot 1553 in the handle, the outer shaft moves relative to the scaffold 1510 allowing the scaffold to move into a deployed configuration or remain within a stowed configuration.

The scaffold 1510 includes a central longitudinal axis 1511 along the inner shaft 1525. The scaffold 1510 includes a proximal end 1513, a distal end 1515, and a plurality of cells 1517. There is also a scaffold transition zone 1518 adjacent to the two or more legs 1519. Each leg 1519 terminates on proximal end in a connection tab 1521. Inner shaft coupler 1530 with key features 1531 to mate with connection tabs 1521 on the proximal end of legs 1519.

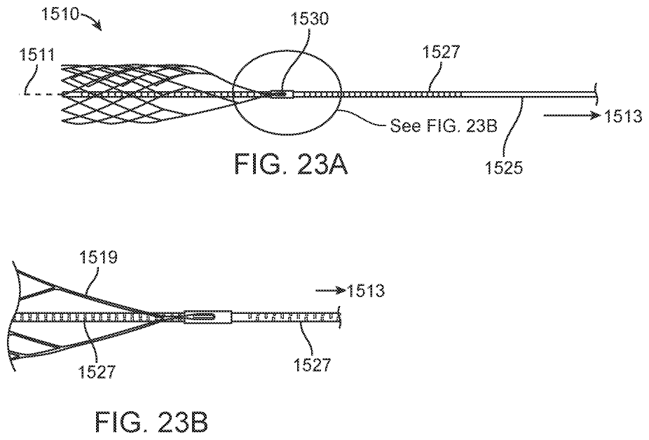

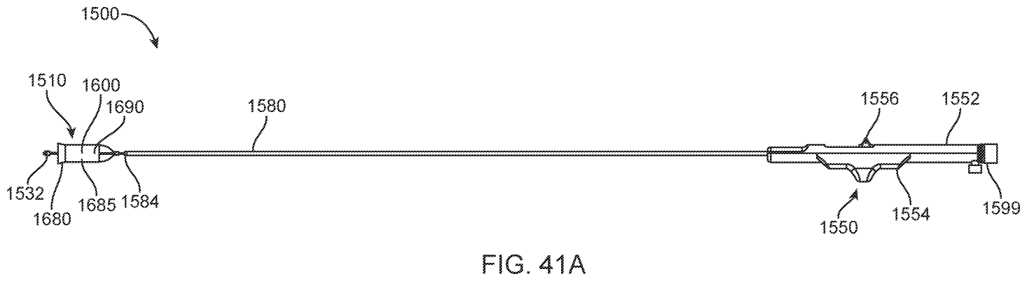

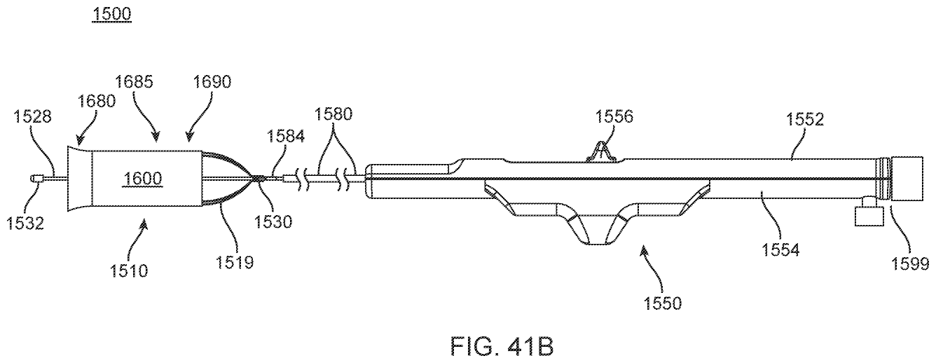

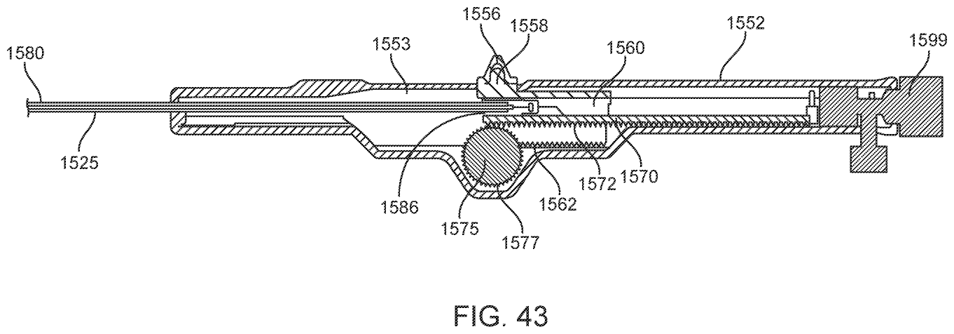

The inner shaft 1525 has a proximal end 1526 and a distal end 1528. The proximal end 1526 is in communication with the hemostasis valve 1599 in the proximal end of handle 1550. (See FIGS. 41A, 41B and 43). The distal most end of the inner shaft 1525 has an atraumatic tip 1532. The inner shaft may be a hypotube suited to provide access to a guidewire via the inner shaft lumen 1597. In one embodiment, the inner shaft has a 0.018″ guidewire lumen. In some embodiments, a series of spiral cuts 1527 are formed along inner shaft 1525 in the proximal end 1526 proximal and distal to the inner shaft coupler 1530. Exemplary positioning of a series of spiral cuts 1527 are illustrated in the various embodiments of FIGS. 23A, 23B, 35, 36, and 37.

FIG. 16 is a distal end view of a bare scaffold 1510 showing three legs 1519 each terminating in a connection tab 1521.

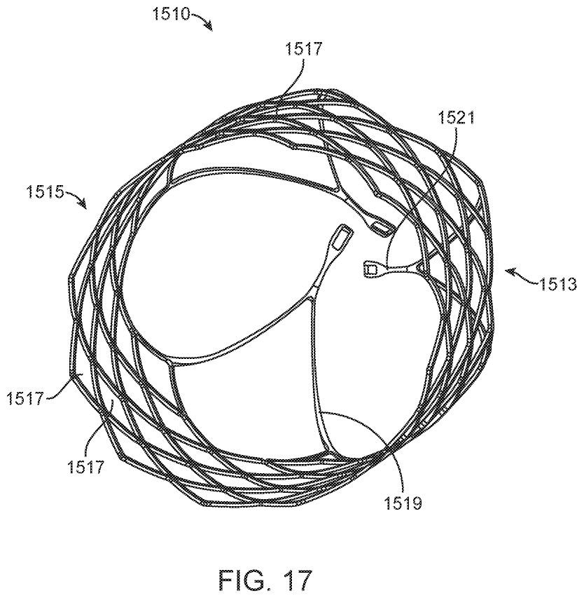

FIG. 17 is an isometric view of the bare scaffold 1510 of FIG. 16.

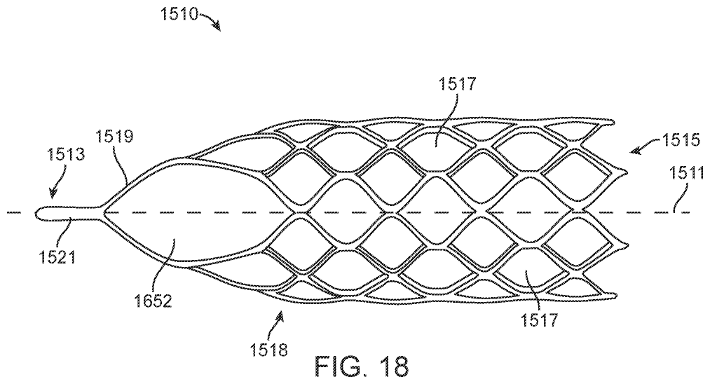

FIG. 18 is a side view of an exemplary scaffold structure having two legs 1519 only one visible in this view.

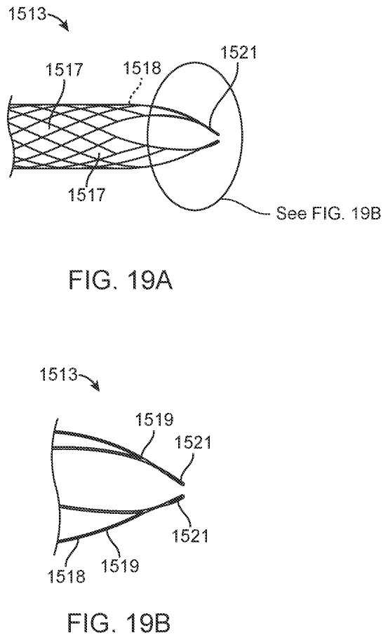

FIG. 19A is a side view of a bare scaffold 1510 with two legs 1519 for attachment to an inner shaft 1525 using an inner shaft coupler 1530.

FIG. 19B is an enlarged view of the connection tab 1521 on the end of each of the two legs 1519 of the scaffold embodiment of FIG. 19A.

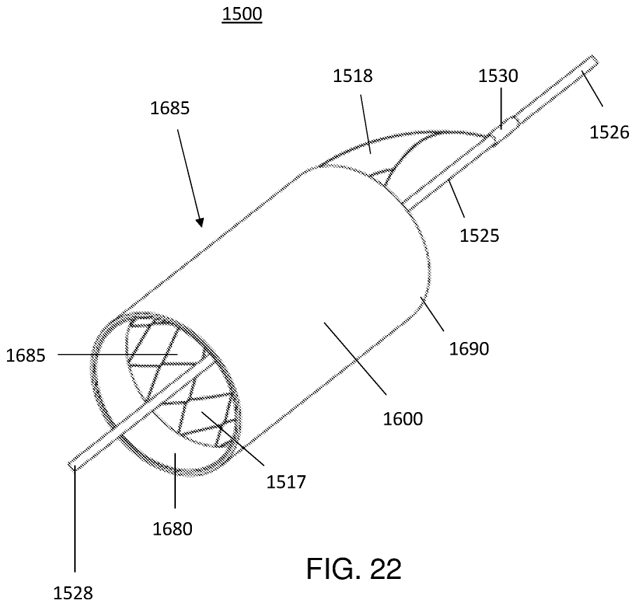

FIGS. 16, 17, 19A, and 19B are distal, isometric, side and enlarged views, respectively, of a laser cut scaffold 1510 of a vascular occlusion device 1500. The covering, coating or membrane 1600 used to at least partially cover the scaffold is omitted to show the details of the scaffold. The scaffold 1510 may be formed from a cut tube of a biocompatible metal using a slot cut or a complex geometry cutting technique to provide a desired cell array as best seen in FIGS. 17, 19A, 21, and 23A. The three legs 1519 structure shown in FIGS. 16, 17, 19A and 19B is provided as an exemplary benefit of the cutting pattern. The three legs could also be wires as in some embodiments the laser cut scaffold is not necessarily a one-piece design. Additionally or optionally, there may be four legs, two legs or one leg. A one leg scaffold embodiment (See FIG. 22). Changes in the number of legs or orientation of the attachment of the legs or leg connection tabs 1521 may be used to also help reduce the diameter of the device. In one aspect, the legs may be connection in a spaced arrangement, off set or staggered in various alternative configurations. In some embodiments, the legs or other structure may be one or more separate pieces designed to address one or more performance features, like collapsing for optimal packing space, or a way to guide the membrane to a collapsed or constrained state.

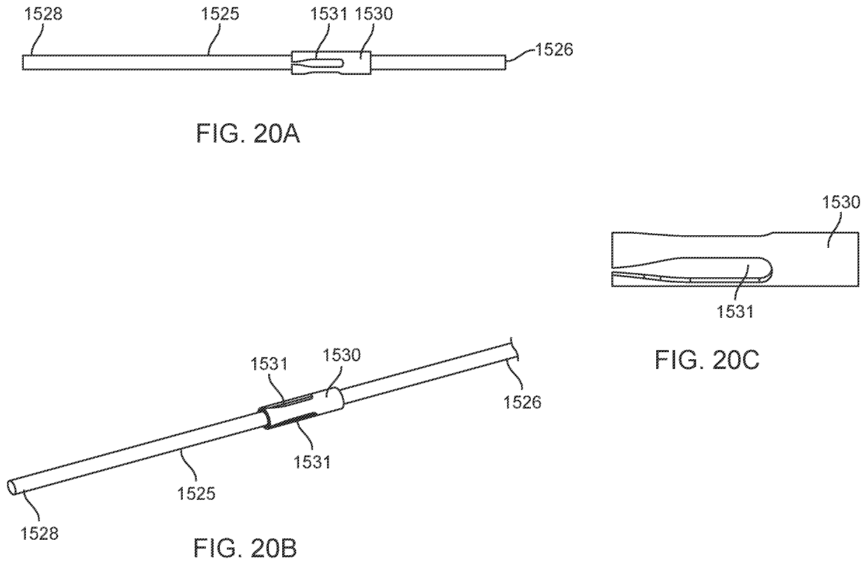

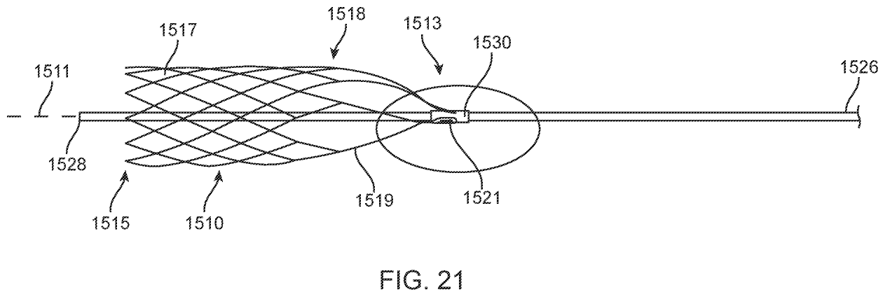

In one embodiment, the scaffold structure 1510 terminates in one end with leg connection tabs 1521 as shown in FIGS. 16, 17 and 19B. In one aspect, the shape of the leg connection tabs 1521 is designed to be complementary with the corresponding slots or complementary key features 1531 formed in an inner shaft coupler 1530. FIGS. 20A, 20B and 20C illustrate isometric and side views respectfully of an exemplary inner shaft coupler 1530 to receive the leg connection tabs 1521. The connection tabs 1521 may be joined to the inner shaft coupler 1530 using any suitable joining technique such as welding or brazing. The final joint appears as shown in FIG. 21 or 23B with the legs 1519 of the scaffold device affixed to the inner shaft coupler 1530 which is affixed to the inner shaft 1525 or hypotube. Additionally or optionally, one or more notches, cuts or slots may be formed in the inner shaft 1525 in one or more locations to improve the flexibility of the inner shaft. In one embodiment, the inner shaft 1525 or hypotube is provided with a pattern of spiral cuts 1527 proximal to the inner shaft coupler 1530, distal to the inner shaft coupler 1530 or proximal and distal to the inner shaft coupler 1530 as needed to provide the desired flexibility in the inner shaft 1525. FIGS. 23A and 23B illustrate an embodiment of an exemplary spiral cut pattern 1527.

FIGS. 20A and 20B are side and perspective views, respectively, of the two key features 1531 of an inner shaft coupler that is attached to an inner shaft.

FIG. 20C is an enlarged view of the shaft coupler of FIGS. 20A and 20B showing the detail of a key feature 1531 shaped to engage with a connection tab 1521 of a scaffold leg 1519.

The inner shaft coupler 1530 is sized for placement on hypotube or central inner shaft 1525. The inner shaft coupler 1530 has keyed or complementary features 1531 to engage with the leg connection tabs 1521 of the scaffold. The proximal end features 1521 of the scaffold legs 1519 are keyed to mate with the inner shaft coupler 1530. The complementary cut outs 1531 used to join the leg tabs 1521 may come in a wide array of shapes and sizes to ensure orientation and position of the scaffold 1510 relative to the central or inner shaft 1525. In some embodiments staggering, offset patterns or other reduction techniques along with keying locations may also help reduce device size.

In the view of FIG. 21, the inner shaft 1525 and scaffold 1510 attached. In this embodiment, there are no spiral cuts 1527 on the inner shaft 1525. The scaffold covering 1600 is removed to show the scaffold detail. Also visible in this view is the joining the leg tabs 1521 and the inner shaft coupler 1530 to the hypotube or inner shaft 1525.

FIG. 22 is a perspective view of an occlusion device 1500 having a single leg 1519 connected to an inner shaft 1525. Terminating the scaffold in a single leg that is in turn coupled to the shaft is another example of a size reduction alternative.

An exemplary vascular occlusion device 1500 includes a handle 1550, an outer shaft 1580, an inner shaft or hypotube 1525 and a covered scaffold coupled to the distal end of the inner shaft 1525. A slider 1556 on the handle 1550 is coupled to the outer shaft 1580. As the slider 1556 moves along a slot 1553 in the handle, the outer shaft moves relative to the scaffold 1510 allowing the scaffold to move into a deployed configuration or remain within a stowed configuration.

The scaffold 1510 includes a central longitudinal axis 1511 along the inner shaft 1525. The scaffold 1510 includes a proximal end 1513, a distal end 1515, and a plurality of cells 1517. There is also a scaffold transition zone 1518 where the scaffold structure transitions to the leg 1519. The leg 1519 terminates on proximal end in a connection tab within a key feature within the inner shaft coupler 1530.

The inner shaft 1525 has a proximal end 1526 and a distal end 1528. The proximal end 1526 is in communication with the hemostasis valve 1599 in the proximal end of handle 1550. (See FIGS. 41A, 41B and 43). The distal most end of the inner shaft 1525 has an atraumatic tip 1532 (not shown in this view).

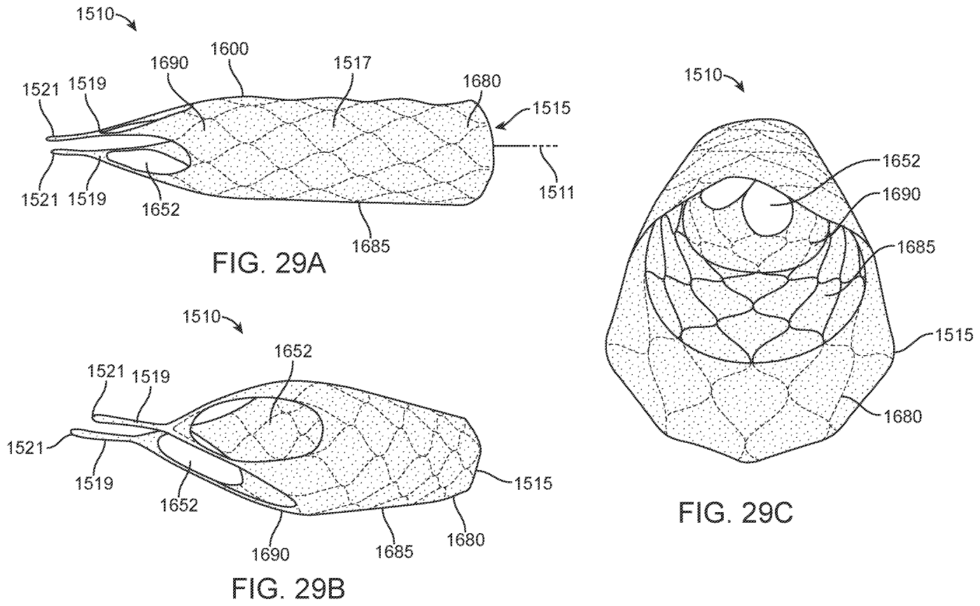

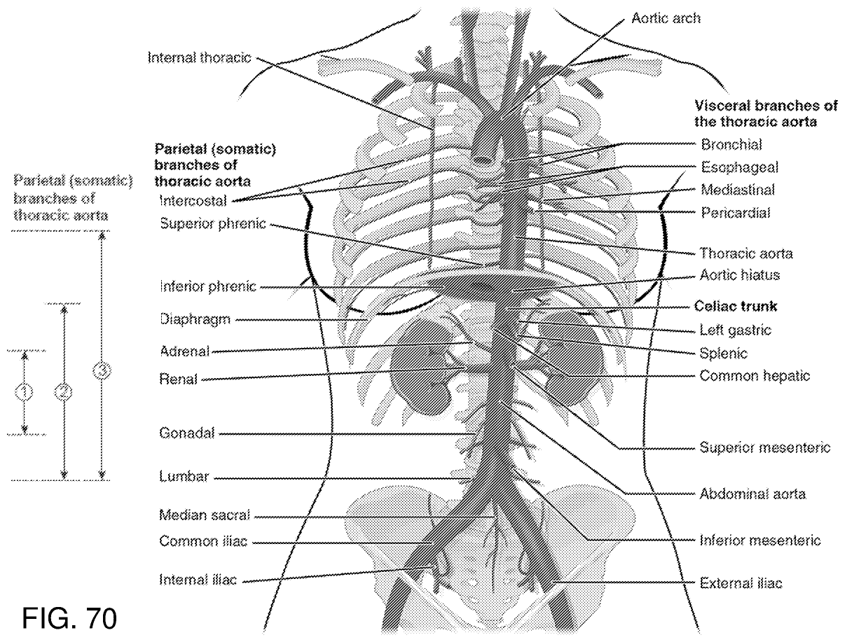

The liner, cover, membrane or scaffold covering 1600 is also visible in this view. As described in greater detail below with regard to FIGS. 29A-29C, a membrane or scaffold covering may be attached to a scaffold in a wide variety of configurations such as: only on an interior aspect of the scaffold, only on an exterior aspect of that scaffold, attached along an entire length of a scaffold, attached only on one, or only on both of the end portions of a scaffold, or on sections of a scaffold or having an unattached covering between two attached portions. The characteristics of the scaffold covering may therefore be adjusted and adapted according to the scenario encountered in a particular temporary occlusion with perfusion setting. FIG. 70, discussed below, describes a range of possible aorta zones or branch vessel or groups of branch vessel configurations.

FIG. 22 illustrates a scaffold covering 1600 having a distal end attachment zone 1680 and a proximal end attachment zone 1690. An unattached zone 1685 is also visible in this view. The amount of overlap on the proximal and distal ends of the covering may range from 2-10 mm. Additionally or optionally, the covering may extend over the leg or legs 1519 to the coupler 1530 or inner shaft 1525. Extending the covering to the connection point of the scaffold legs to the inner shaft or the shaft of the occlusion device 1525 aids in movement of the outer sheath to transition the covered scaffold from the deployed condition as shown in FIG. 22 and a stowed condition where the covered scaffold is within the outer sheath.

FIGS. 23A and 23B illustrate details of a series of spiral cuts 1527 made in the inner shaft 1525 proximal and distal to the inner shaft coupler 1530. Also visible in this view is the joining of the leg tabs 1521 and the inner shaft coupler 1530 to the hypotube or inner shaft 1525.

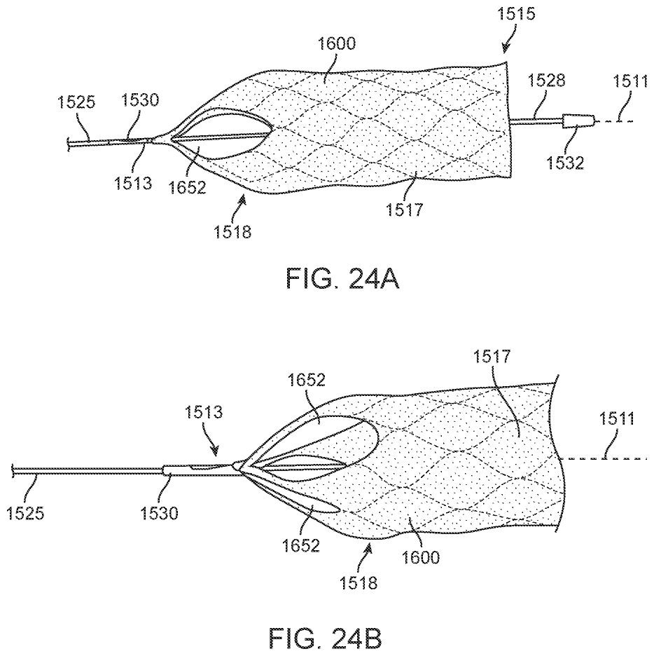

FIG. 24A is an exemplary view of a covered scaffold in a deployed configuration connected to the inner shaft. Openings 1652 cut around the legs and the atraumatic tip 1532 of the inner shaft are also visible in this view.

FIG. 24B is an enlarged view of the proximal end of the covered scaffold in FIG. 24A showing the covering 1600 on the legs 1519 extends into the inner shaft coupler 1530. This view also shows the cut outs 1652 formed in the covering 1600 between the covered legs of the scaffold.

FIGS. 24A and 24B include the one or more openings 1652 are formed in the covering. The openings 1652 in FIGS. 24A and 24B allow for the scaffold transition zone 1518 and the legs 1519 to remain covered while providing large openings to permit perfusion blood flow through the covered scaffold.

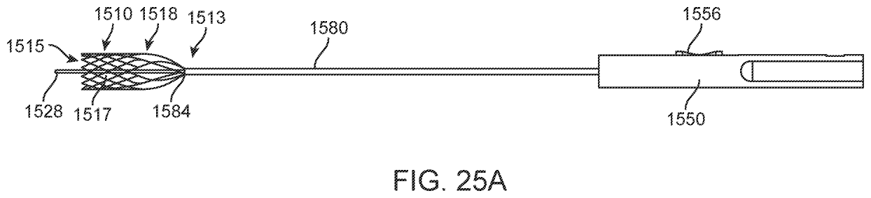

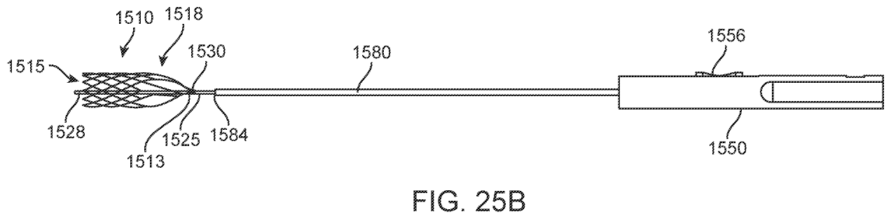

FIG. 25A is a side view of a vascular occlusion device shown without any cover. In this view, the outer shaft is withdrawn using the slider on the handle to position the distal end of the outer shaft at the proximal end of the scaffold. In this embodiment, in the deployed configuration the outer shaft is withdrawn proximal to the scaffold transition zone with the inner shaft coupler remaining within and covered by the outer shaft.

FIG. 25B is a side view of a vascular occlusion device of FIG. 25A. The slider on the handle is in a proximal position to withdraw the outer shaft or sheath from the scaffold allowing the scaffold to transition from a stowed configuration to deployed configuration as illustrated. In this embodiment, in the deployed configuration the outer shaft is withdrawn proximal to the inner shaft coupler.

FIG. 25A is a side view of an exemplary vascular occlusion device, with covering removed to show scaffold detail. There is a handle 1550 coupled to the inner and outer shafts 1525, 1580. An outer shaft or sheath 1580 is disposed over the inner shaft and the scaffold structure and is moveable by a slider on the handle. A slider in the handle controls the position of the outer shaft 1580 or sheath relative to the inner shaft 1525 and scaffold 1510. The slider knob 1556 is shown in a proximal position on the handle. In this position the sheath is moved proximally towards the handle thereby allowing the scaffold to transition from the stowed configuration to the deployed configuration. In the deployed configuration the vascular occlusion device engages the vessel interior wall to seal partially or completely as desired by the amount of occlusion and distal perfusion to be achieved by a specific embodiment. FIG. 25B is another view of the device in FIG. 25A with the guide partially withdrawn to show the detail of the spiral cuts on the hypotube proximal and distal to the mating collar.

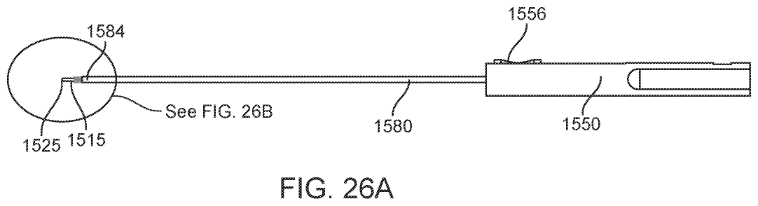

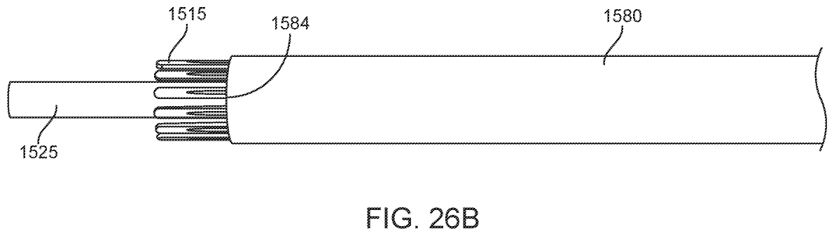

FIG. 26A is a side view of a vascular occlusion device in a stowed condition with the outer shaft withdrawn slightly to show the stowed distal end of the scaffold as best seen in the enlarged view of FIG. 26B. The slider on the handle is withdrawn slightly from the distal most position on the handle to only slightly withdraw the outer sheath to the illustrated position. Continued proximal movement of the slider will continue to withdraw the outer shaft or sheath 1580 from the scaffold allowing the scaffold to transition from a stowed configuration to deployed configuration.

FIG. 26A shows an exemplary vascular occlusion device in a stowed configuration. The slider knob is in a distal position on the handle and the sheath is covering substantially all of the scaffold device. Slider knob 1556 is used to control position of sheath or outer shaft 1580—shown in position to maintain sheath over the scaffold which retains the scaffold 1510 in a stowed configuration. FIG. 26B is an enlarged portion of the distal end of the device shown in FIG. 26A. In the view of FIG. 26B, the distal end of the sheath terminates with the distal most end of the scaffold and the terminal end of the hypotube exposed. Other sheath positions are possible where the scaffold is maintained in a stowed configuration an only the terminal end or portion of the hypotube is exposed. Optionally, the sheath may be selected such that none of the hypotube or the scaffold is showing. In additional embodiments, the sheath is positioned relative to the stowed condition of the scaffold to allow for ease of movement of the slider to deploy the scaffold.

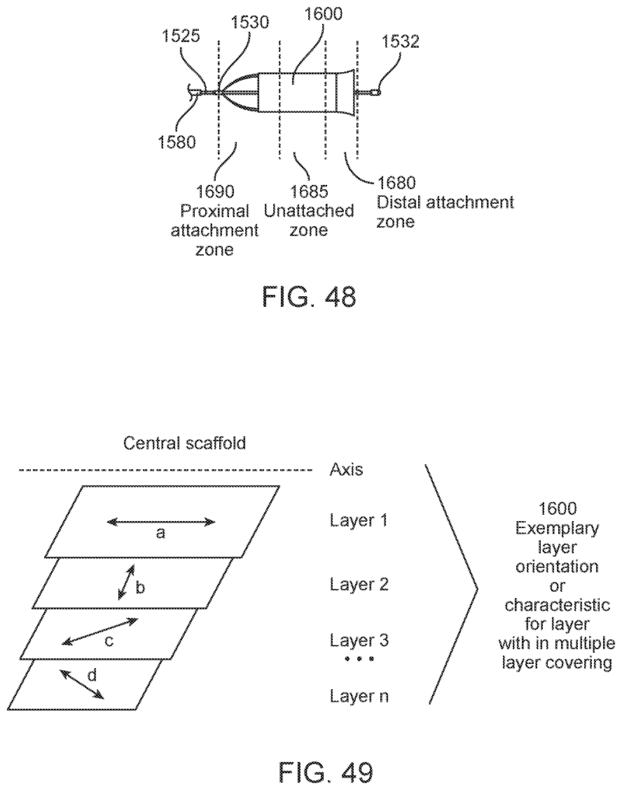

It is to be appreciated that a number of different scaffold coverings 1600 may be provided that will provide for at least partial occlusion of the peripheral vessels while simultaneously providing for perfusion blood flow to the vessels and structures distal to the vascular occlusion device. Additional details of the scaffold covering 1600 are described below with regard to FIGS. 48 and 49.

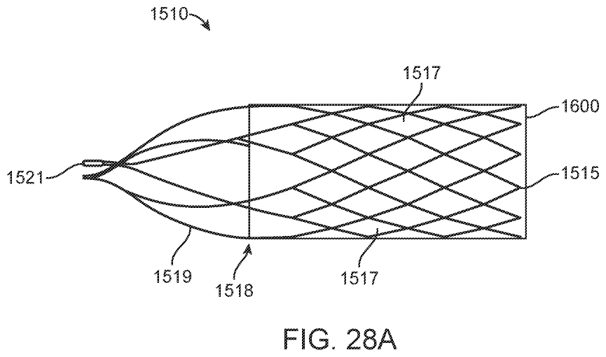

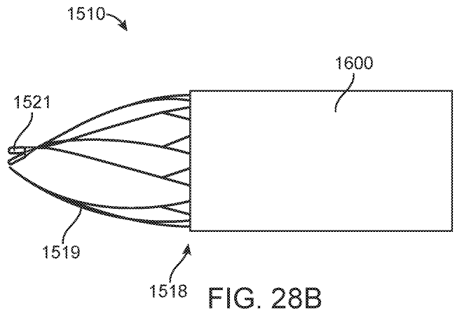

FIGS. 27, 28A and 28B are isometric and side views respectively of a scaffold device that covers most all of the scaffold structure from the distal end 1513 to the proximal end 1513 including the legs 1519 and connection tabs 1521 in some embodiments. When deployed within the vasculature the covered portion of the scaffold is one factor used to refine and define occlusion characteristics of the device. While once the covered scaffold is deployed in the vasculature, the blood flow is directed into the interior of the scaffold through the open central portion along the central longitudinal axis 1511 of the scaffold as well as through other uncovered or only partially covered scaffold portions are also used to refine and define the vascular occlusion device perfusion characteristics. Adjusting the relative amount and type of covering and open scaffold portions enables a wide array of occlusion and perfusion device characteristics. In some embodiments, a covered scaffold in the cylindrical scaffold portion extends from distal most portion of scaffold but covering stops before transition to legs in the scaffold transition zone 1518. An interior wall of the scaffold covering or membrane is also visible in the view of FIG. 27.

In some alternative embodiments, all of the scaffold structure but the legs are covered by a suitable scaffold covering 1600. The distal end to a portion of the scaffold where the legs are extending towards the coupling device as detailed above. In this way, some scaffold embodiments deploy into much like a tube or barrel shape which extends along the adjacent vessel wall where the scaffold is deployed. Any peripheral vessel along the covered portion of the main vessel will be partially or fully occluded. The covering extends from the distal end of the scaffold structure to the proximal end where the scaffold structure transitions to the legs and then tabs for joining to the coupling on the inner tube. The scaffold covering 1600 is shown as transparent in the view of FIG. 28A in show the detail of the scaffold structure in relation to the size of scaffold covering used. The scaffold covering 1600 material may be transparent or opaque. An opaque membrane or scaffold covering is shown in FIG. 28B.

FIG. 29A is a side view of a covered scaffold embodiment having two legs for attachment to the central shaft. This covered scaffold embodiment includes a proximal scaffold attachment zone 1690, a distal scaffold attachment zone 1680 and a central covering portion that is unattached to the scaffold (unattached zone 1685). The covering 1600 on the legs to the connection tabs and the distal openings are also seen in this view.

FIG. 29B is a perspective view of the proximal end of the covered scaffold of FIG. 29A. The proximal attachment zone is visible in this view through a distal opening.

FIG. 29C is a perspective view of the distal end of the covered scaffold in FIG. 29A. The proximal attachment zone, the distal attachment zone and the distal openings are visible in this view. In one embodiment, the distal and proximal attachment portions are formed by folding the scaffold covering over the proximal and distal ends of the scaffold. FIG. 29 also illustrates a distal end 1620 that includes a distal folded portion 1622 over the distal end of the scaffold 1515. Similarly, a proximal end 1630 may include a proximal folded portion 1632 over the proximal end of the scaffold 1513, optionally including covering the legs 1519 and optionally including covering the connection tabs 1521.

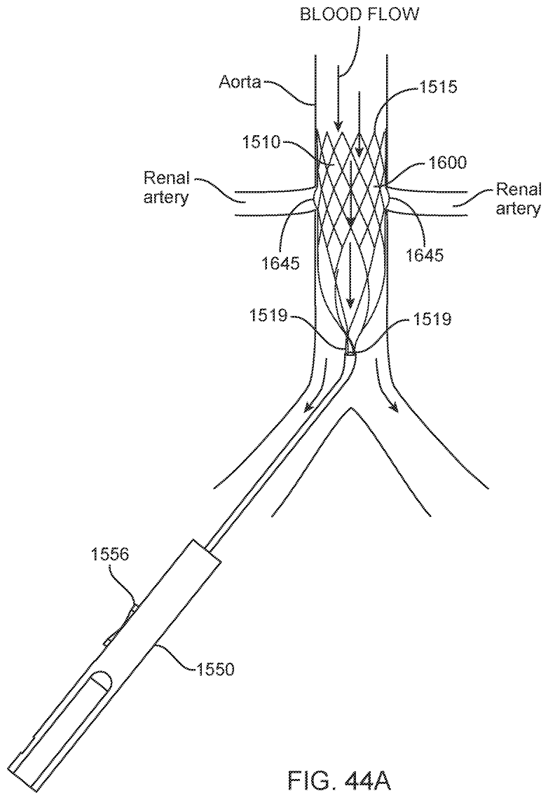

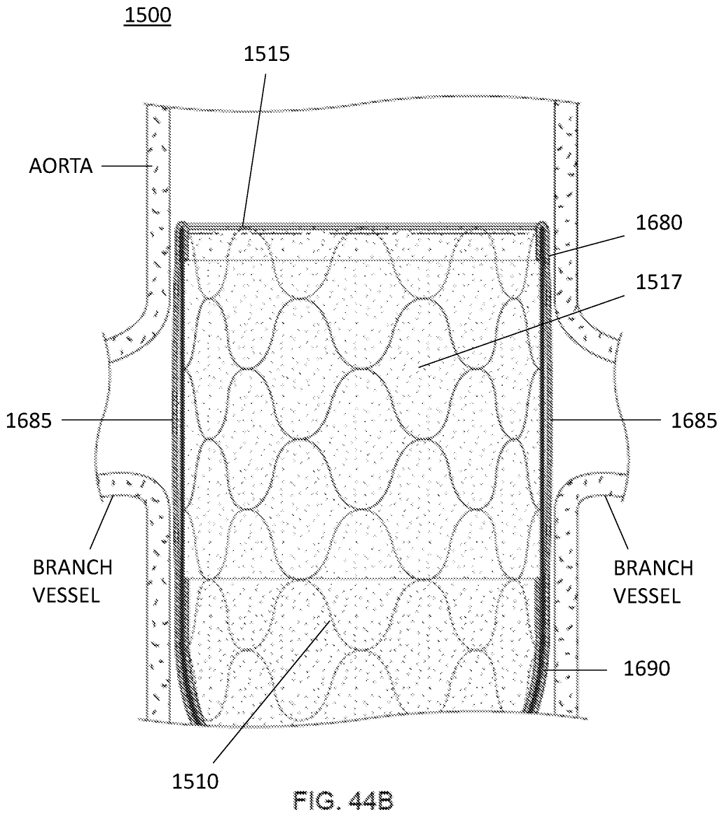

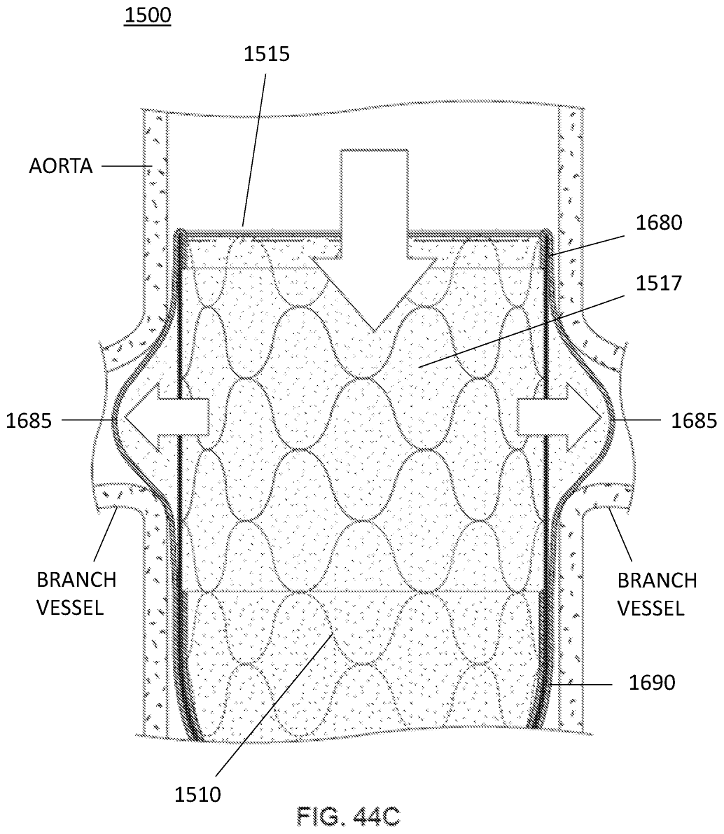

FIGS. 29A, 29B and 29C include the one or more openings 1652 are formed in the scaffold covering 1600. The openings 1652 best seen in FIGS. 29A and 29B allow for the scaffold transition zone 1518 and both of the legs 1519 to remain covered while providing large openings to permit perfusion blood flow through the covered scaffold. (See additionally FIGS. 44B and 44C).

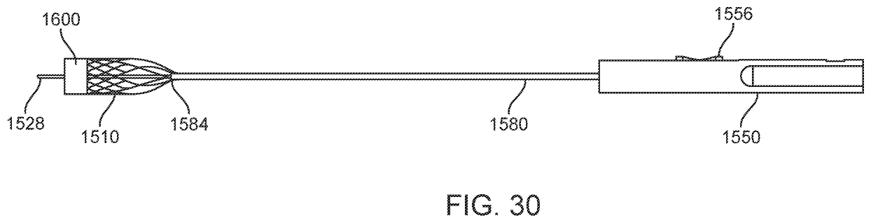

FIG. 30 is a side view of an exemplary vascular occlusion device, with a 20% covering of the scaffold. There is a handle coupled to a hypotube. A sheath is disposed over the hypotube and coupled to the handle. A slider knob in the handle controls the position of the sheath relative to the hypotube and scaffold device. The slider knob is shown in a proximal position on the handle. In this position the sheath is moved proximally towards the handle thereby allowing the scaffold to transition from the stowed configuration to the deployed configuration. In the deployed configuration the vascular occlusion device engages the vessel interior wall to seal partially or completely as desired by the amount of occlusion and distal perfusion to be achieved by a specific embodiment. Full Device 20% covered scaffold. Distal end of the covering aligns to the distal most portion of the scaffold structure. Slider to control position of sheath—shown in position to retract the sheath. Proximal end of the covering extends along the scaffold structure so that approximately 20% of the scaffold structure is covered. When deployed within the vasculature the covered portion of the scaffold is one factor used to refine and define occlusion characteristics of the device while the generally open central portion or other uncovered scaffold portions refine and define the device perfusion characteristics. Adjusting the relative amount and type of covering and open scaffold portions enables a wide array of occlusion and perfusion device characteristics (FIG. 30).

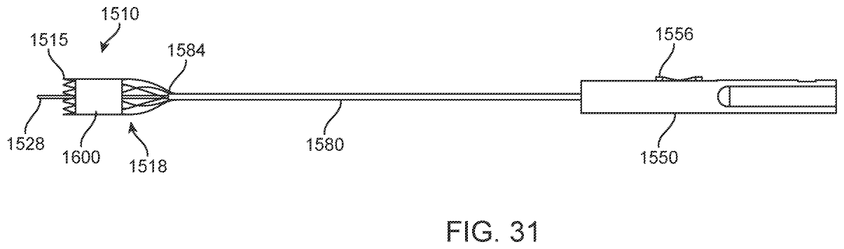

FIG. 31 is a side view of an embodiment of a vascular occlusion device in a deployed condition having a 50% scaffold covering. The slider on the handle is in a proximal position to withdraw the outer shaft or sheath 1580 from the scaffold allowing the scaffold to transition from a stowed configuration to deployed configuration as illustrated. The 50% scaffold covering distal end aligns proximal to the scaffold distal end and extends proximally along the longitudinal length of the scaffold to cover approximately 50% of the overall length of the scaffold.

FIG. 31 is a side view of an exemplary vascular occlusion device, with a 50% covering of the scaffold. There is a handle coupled to a hypotube. A sheath is disposed over the hypotube and coupled to the handle. A slider knob in the handle controls the position of the sheath relative to the hypotube and scaffold device. The slider knob is shown in a proximal position on the handle. In this position the sheath is moved proximally towards the handle thereby allowing the scaffold to transition from the stowed configuration to the deployed configuration. In the deployed configuration the vascular occlusion device engages the vessel interior wall to seal partially or completely as desired by the amount of occlusion and distal perfusion to be achieved by a specific embodiment. Full device—50% coverage centered. When deployed within the vasculature the covered portion of the scaffold is one factor used to refine and define occlusion characteristics of the device while the generally open central portion or other uncovered scaffold portions refine and define the device perfusion characteristics. Adjusting the relative amount and type of covering and open scaffold portions enables a wide array of occlusion and perfusion device characteristics. Distal end of the covering is spaced back proximally from the distal most end (the crowns) of the scaffold structure. Slider to control position of sheath—shown in position to retract the sheath. Proximal end of the covering extends along the scaffold structure so that approximately 50% of the scaffold structure is covered. The distal end of the covering is positioned along the scaffold structure and distal to the scaffold transition zone (FIG. 31).

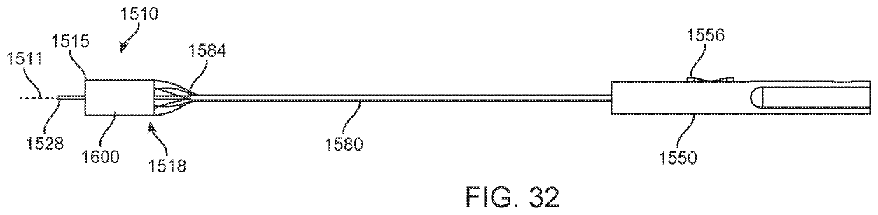

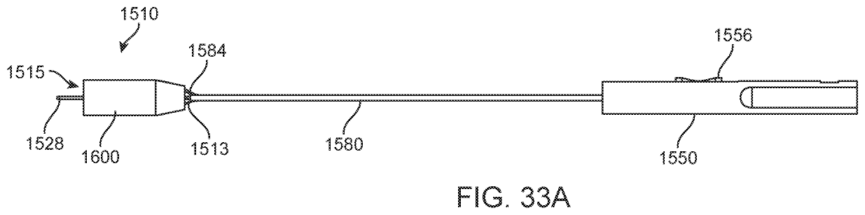

FIG. 32 is a side view of an embodiment of a vascular occlusion device in a deployed condition having an 80% scaffold covering. The slider on the handle is in a proximal position to withdraw the outer shaft or sheath 1580 from the scaffold allowing the scaffold to transition from a stowed configuration to deployed configuration as illustrated. The 80% scaffold covering distal end aligns with the scaffold distal end and extends proximally along the longitudinal length of the scaffold to cover approximately 80% of the overall length of the scaffold.

FIG. 32 is a side view of an exemplary vascular occlusion device, with an 80% covering of the scaffold. There is a handle coupled to a hypotube. A sheath is disposed over the hypotube and coupled to the handle. A slider knob in the handle controls the position of the sheath relative to the hypotube and scaffold device. The slider knob is shown in a proximal position on the handle. In this position the sheath is moved proximally towards the handle thereby allowing the scaffold to transition from the stowed configuration to the deployed configuration. In the deployed configuration the vascular occlusion device engages the vessel interior wall to seal partially or completely as desired by the amount of occlusion and distal perfusion to be achieved by a specific embodiment. Full device—80% coverage. Distal end of the covering aligns to the distal most portion of the scaffold structure. Slider to control position of sheath—shown in position to retract the sheath. Proximal end of the covering extends along the scaffold structure so that approximately 80% of the scaffold structure is covered. The distal end of the covering is positioned along the scaffold structure and terminates at the scaffold transition zone. The legs are uncovered. When deployed within the vasculature the covered portion of the scaffold is one factor used to refine and define occlusion characteristics of the device while the generally open central portion or other uncovered scaffold portions refine and define the device perfusion characteristics. Adjusting the relative amount and type of covering and open scaffold portions enables a wide array of occlusion and perfusion device characteristics (FIG. 32).

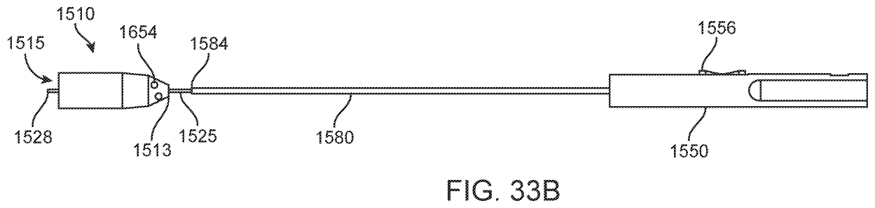

FIG. 33A is a side view of an embodiment of a vascular occlusion device in a deployed condition having an 100% scaffold covering. The 100% scaffold covering distal end aligns with the scaffold distal end and extends proximally along the longitudinal length of the scaffold to cover approximately 100% of the overall length of the scaffold with the exception of a small portion of the end of the device as shown. The slider on the handle is in a proximal position to withdraw the outer shaft or sheath from the scaffold allowing the scaffold to transition from a stowed configuration to deployed configuration as illustrated.

FIG. 33A is side view of a nearly completely covered vascular occlusion device. The embodiment of FIG. 33A is an exemplary vascular occlusion device, with a nearly 100% covering of the scaffold. The amount of distal perfusion may be adjusted by the gap between the covering around the proximal end of the device and the hypotube. There is a handle coupled to a hypotube. A sheath is disposed over the hypotube and coupled to the handle. A slider knob in the handle controls the position of the sheath relative to the hypotube and scaffold device. The slider knob is shown in a proximal position on the handle. In this position the sheath is moved proximally towards the handle thereby allowing the scaffold to transition from the stowed configuration to the deployed configuration. In the deployed configuration the vascular occlusion device engages the vessel interior wall to seal partially or completely as desired by the amount of occlusion and distal perfusion to be achieved by a specific embodiment. Full device—100% coverage scaffold with central flow through distal perfusion capability. Distal end of the covering aligns to the distal most portion of the scaffold structure. When deployed within the vasculature the covered portion of the scaffold is one factor used to refine and define occlusion characteristics of the device while the generally open central portion or other uncovered scaffold portions refine and define the device perfusion characteristics. Adjusting the relative amount and type of covering and open scaffold portions enables a wide array of occlusion and perfusion device characteristics. Proximal end of the covering extends along the scaffold structure so that approximately all of the scaffold structure is covered. The distal end of the covering is positioned along the scaffold structure and the transition portion. The legs are covered. The covering terminates along the legs leaving an opening of larger diameter than the sheath which allows a central distal perfusion flow. Small opening here—end is not closed. Slider to control position of sheath—shown in position to retract the sheath (FIG. 33A).

FIG. 33B is side view of a nearly completely covered vascular occlusion device. The embodiment of FIG. 33B is similar to that of FIG. 33A in that the vascular occlusion device has a nearly 100% covering of the scaffold. As with the embodiment of FIG. 33A, the amount of distal perfusion may be adjusted by the gap between the covering around the proximal end of the device and the hypotube. Additionally, the embodiment of FIG. 33B includes one or more apertures in the membrane or covering to further adjust the amount of distal perfusion.

FIG. 33B is a side view of an embodiment of a vascular occlusion device in a deployed condition having an 100% scaffold covering similar to FIG. 33A. The slider on the handle is in a proximal position to withdraw the outer shaft or sheath from the scaffold allowing the scaffold to transition from a stowed configuration to deployed configuration as illustrated. This embodiment illustrates a plurality of openings formed in the proximal end of the covering within the scaffold transition zone. The 100% scaffold covering distal end aligns with the scaffold distal end and extends proximally along the longitudinal length of the scaffold to cover approximately 100% of the overall length of the scaffold.

Similar to other embodiments, there is a handle on the proximal end of the vascular occlusion device. A sheath or outer shaft is disposed over the inner shaft or hypotube and coupled to the handle. A slider knob in the handle controls the position of the sheath relative to the hypotube and scaffold device. In this view, the slider knob is shown in a proximal position on the handle. In this position the sheath is moved proximally towards the handle thereby allowing the scaffold to transition from the stowed configuration to the deployed configuration. In the deployed configuration the vascular occlusion device engages the vessel interior wall to seal partially or completely as desired by the amount of occlusion and distal perfusion to be achieved by a specific embodiment.

In this embodiment, the full scaffold device is covered completely or considered a 100% coverage of the scaffold with the scaffold covering 1600. Advantageously, the directed flow through or distal perfusion capability is adjustable by the number, size and arrangement of the openings 1654 as shown in FIG. 33B. The amount of perfusion provided by the vascular occlusion device being determined by shape, size, pattern and location of perfusion openings or apertures 1654. While illustrated in the proximal end of the covered scaffold the apertures 1654 may be positioned in other portions of the scaffold covering 1600 depending on the clinical scenario where the vascular occlusion device is employed. As such, it is to be appreciated that a scaffold covering 1600 or other suitable biocompatible vascular membrane includes one or more or a pattern of apertures 1654 that are shaped, sized or positioned relative to the scaffold structure to modify the amount of distal perfusion. Additionally or optionally, the suitable membrane or scaffold covering 1600 may include apertures 1654 having one or more regular or irregular geometric shapes arranged in a continuous or discontinuous pattern which is selected to adapt the distal perfusion flow profile of an embodiment of the vascular occlusion device.

Distal end of the covering aligns to the distal most portion of the scaffold structure. When deployed within the vasculature the covered portion of the scaffold is one factor used to refine and define occlusion characteristics of the device while the generally open central portion or other uncovered scaffold portions refine and define the device perfusion characteristics. Adjusting the relative amount and type of covering and open scaffold portions enables a wide array of occlusion and perfusion device characteristics. Proximal end of the covering extends along the scaffold structure so that approximately all of the scaffold structure is covered. The distal end of the covering is positioned along the scaffold structure and the transition portion. The legs are covered. Distal perfusion is provided by flow through perfusion apertures formed in the membrane covering. Perfusion apertures may be provided as a pattern of small openings in the scaffold covering. Slider is used to control position of over shaft or sheath and is shown in position to retract the outer shaft.

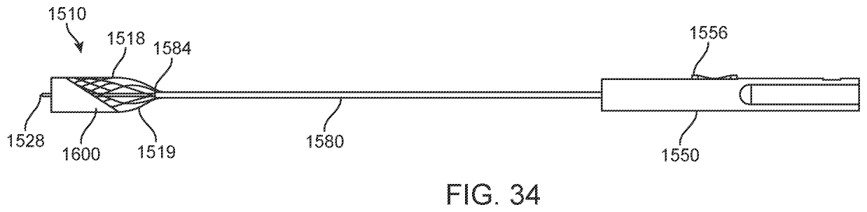

FIG. 34 is a side view of an embodiment of a vascular occlusion device in a deployed condition having a tapered scaffold covering of a partial cylindrical section. The slider on the handle is in a proximal position to withdraw the outer shaft or sheath from the scaffold allowing the scaffold to transition from a stowed configuration to deployed configuration as illustrated. The tapered scaffold covering distal end aligns with the scaffold distal end and extends proximally along the longitudinal length of the scaffold to various distal positions according to the overall covering shape. In this view the exemplary shaped covering extends over only a few cells of the scaffold in the top portion while covering most all of the cells and nearly reaching the scaffold transition zone in the bottom portion.

FIG. 34 is side view of a partially completely covered vascular occlusion device. The embodiment of FIG. 34 illustrates how the shape of the membrane or covering may be modified in order to adjust the amount of distal perfusion. In the embodiment of FIG. 34 there is a tapered cylindrical membrane attached to the scaffold. Other partially covered membrane shapes may be used including combinations of regular and irregular shapes to adapt the membrane and scaffold structure to the specific anatomical environment or a desired occlusion and distal perfusion flow profile. As such, the amount of distal perfusion may be adjusted by the relative amounts of covered and exposed scaffold. Additionally or optionally, the shaped membrane embodiment of FIG. 34 may include one or more apertures in the membrane or covering to further adjust the amount of distal perfusion. There is a handle coupled to the inner and outer shafts as described herein. The slider knob is shown in a proximal position on the handle. In this position the sheath is moved proximally towards the handle thereby allowing the scaffold to transition from the stowed configuration to the deployed configuration. In the deployed configuration the vascular occlusion device engages the vessel interior wall to seal partially or completely as desired by the amount of occlusion and distal perfusion to be achieved by a specific embodiment.

Occlusion and perfusion device embodiment with a partial scaffold covering or membrane. In some embodiments, the scaffold covering 1600 or membrane may also cover only a portion of the scaffold in any of a variety of shapes such as the cut cylinder shape shown here. Other geometric shapes or irregular shapes may be employed for membrane overall shapes which will enable a wide array of different and controllable occlusion parameters along with a variety of simultaneous distal perfusion capabilities. When deployed within the vasculature the covered portion of the scaffold is one factor used to refine and define occlusion characteristics of the device while the generally open central portion or other uncovered scaffold portions refine and define the device perfusion characteristics. Adjusting the relative amount and type of covering and open scaffold portions enables a wide array of occlusion and perfusion device characteristics (see FIG. 34).

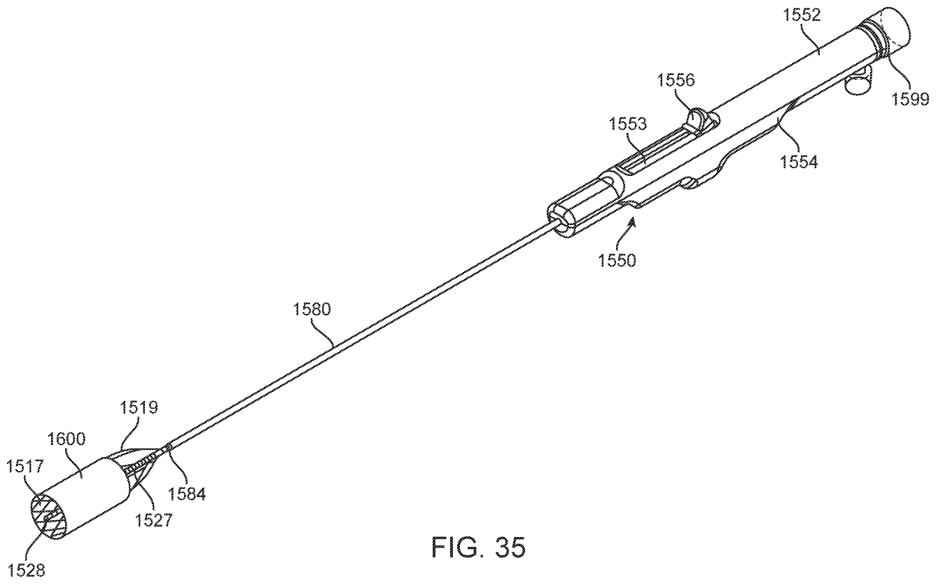

FIG. 35 is a perspective view of an embodiment of a vascular occlusion device in a deployed configuration having a scaffold covering extending from the distal end of the scaffold to the scaffold transition zone. The slider on the handle is in a proximal position to withdraw the outer shaft or sheath from the scaffold allowing the scaffold to transition from a stowed configuration to deployed configuration as illustrated. A portion of the distal attachment zone is visible in this view along with a section of the spiral cut inner shaft.

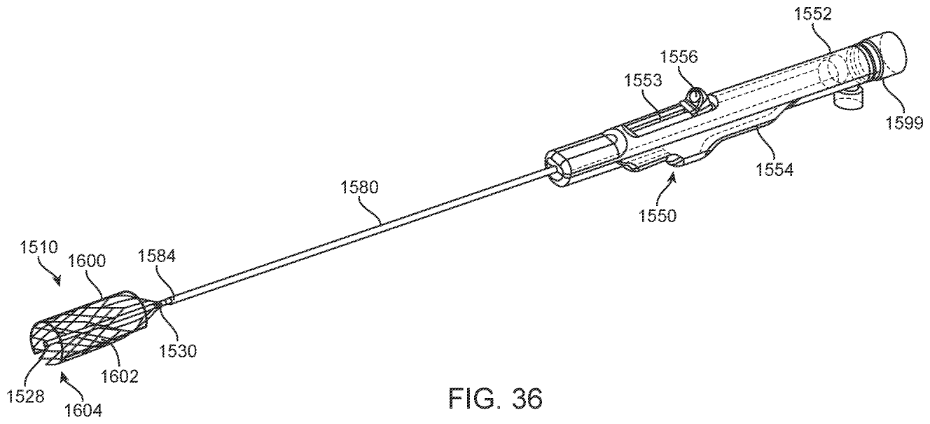

FIG. 36 is a perspective view of an embodiment of a vascular occlusion device in a deployed configuration having a scaffold covering extending from the distal end of the scaffold to the scaffold transition zone for about 270 degrees of the scaffold circumference. A portion of the scaffold along the bottom section remains uncovered as shown. The slider on the handle is in a proximal position to withdraw the outer shaft or sheath from the scaffold allowing the scaffold to transition from a stowed configuration to deployed configuration as illustrated. A portion of the distal attachment zone is visible in this view along with a section of the spiral cut inner shaft.

The vascular occlusion device of FIG. 36 is an exemplary embodiment of an occlusion device where the scaffold covering extends partially circumferentially about the scaffold structure. As seen in this view, the scaffold covering extends from the distal attachment zone 1680 to the proximal attachment zone 1690 and also includes an uncovered scaffold structure 1604. In this exemplary embodiment, the scaffold covering 1600 has a partial circumferential portion 1602 that extends partially circumferentially about 270 degrees of the scaffold structure from the distal attachment zone to the proximal attachment zone with the uncovered portion 1604 along the bottom of the scaffold. An embodiment such as this would be useful for peripheral vessels that are located on the sidewalls or upper portion of the vessel.

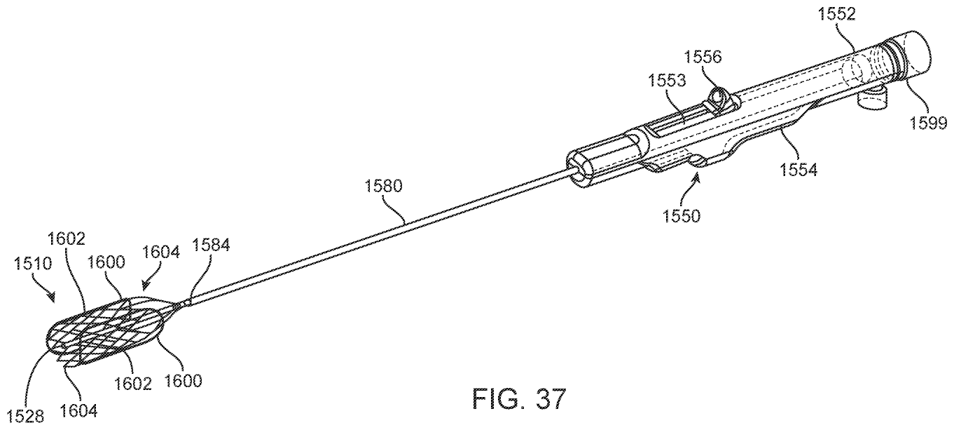

FIG. 37 is a perspective view of an embodiment of a vascular occlusion device in a deployed configuration having a pair of scaffold covering sections 1602 extending from the distal end of the scaffold to the scaffold transition zone for about 45 degrees of the scaffold circumference. Upper and lower uncovered scaffold portions 1604 are along the top and bottom of the scaffold. The portions 1604 of the scaffold along the top and bottom section remains uncovered as shown. The slider 1556 on the handle 1550 is in a proximal position to withdraw the outer shaft 1580 or sheath from the scaffold allowing the scaffold to transition from a stowed configuration to deployed configuration as illustrated. An embodiment such as this would be useful for peripheral vessels that are located on the sidewalls of the vessel.

A portion of the distal and proximal attachment zones of one of the scaffold covering sections is visible in this view along with a section of the spiral cut inner shaft.

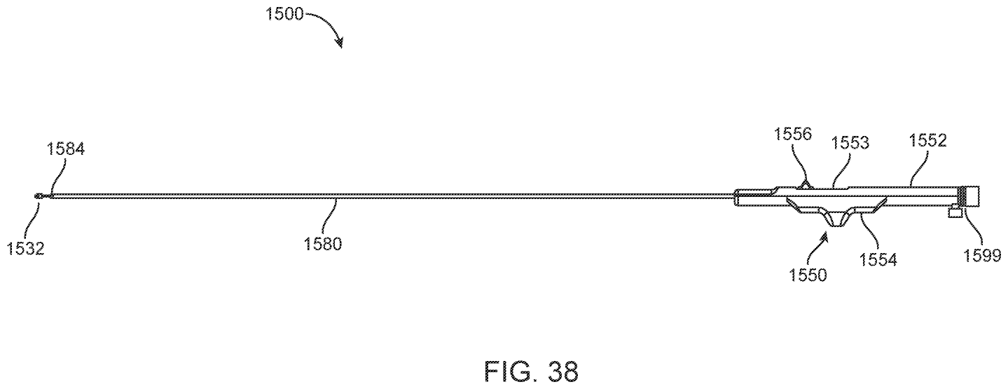

FIG. 38 is a perspective view of an embodiment of a vascular occlusion device in a stowed configuration. The slider on the handle is in a distal position with the outer shaft or sheath over the covered scaffold and maintaining it in a stowed configuration.

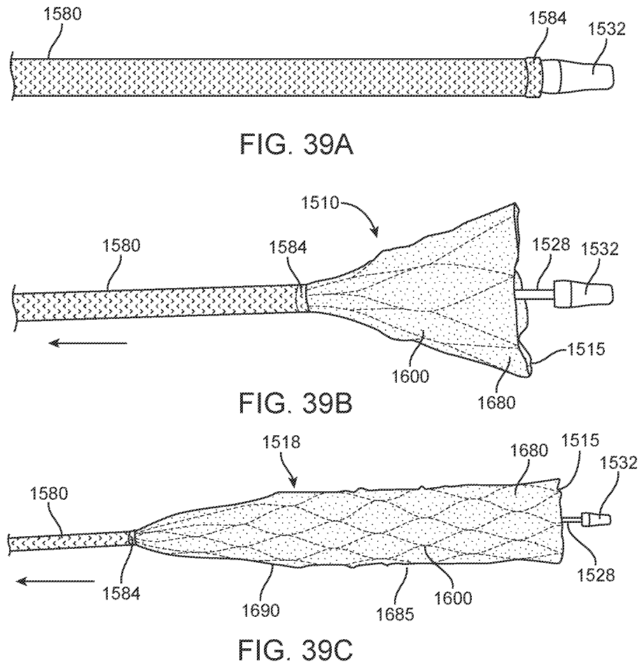

FIG. 39A is an enlarged view of the distal end of the stowed vascular occlusion device of FIG. 38.

FIG. 39B is the enlarged view of FIG. 39A showing the proximal movement (indicated by the arrows) of the distal end of the outer shaft 1580 or sheath as the slider on the handle advances proximally. The distal end of the covered scaffold and a portion of the distal attachment zone 1680 is also shown in this view.

FIG. 39C is the view of FIG. 39B showing the result of continued proximal movement of the slider (indicated by the arrows for the movement of the outer shaft 1580) and corresponding proximal movement of the outer shaft allowing more of the covered scaffold to transition into the deployed configuration.

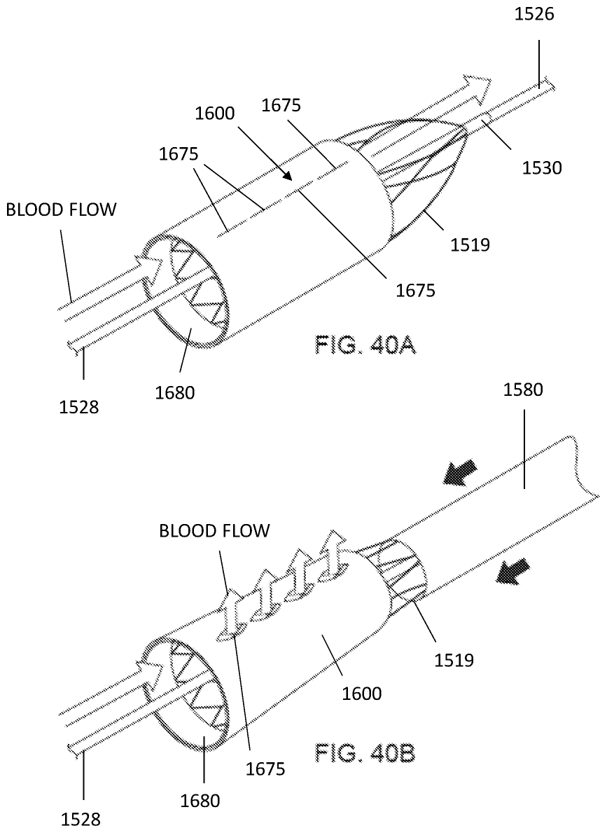

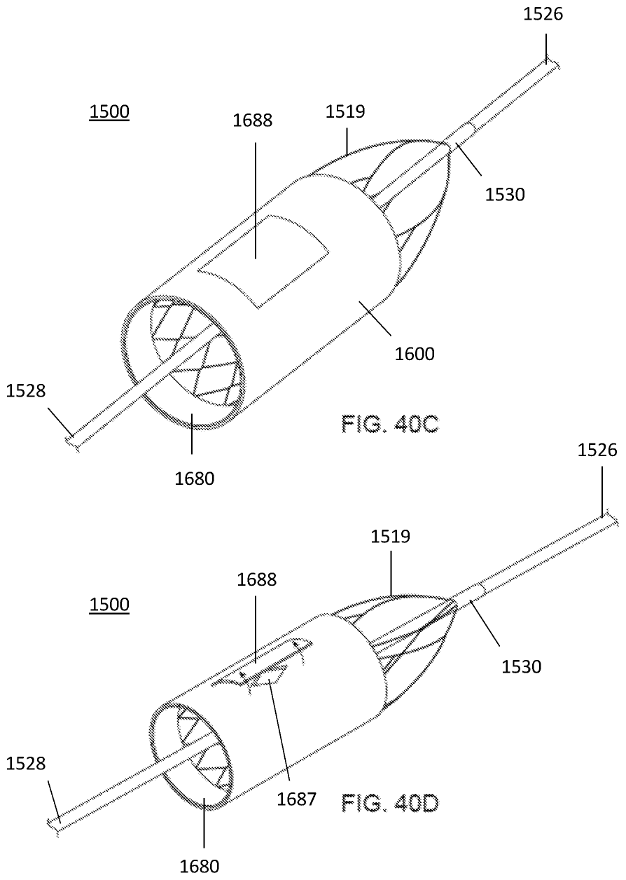

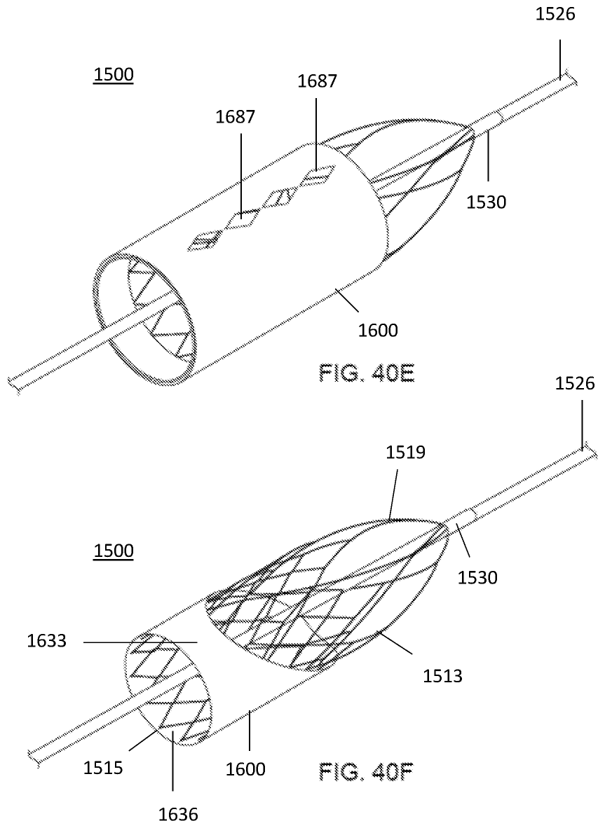

FIG. 40A is a perspective view of an occlusion device 1500 having a series of pressure release slits 1675 along an upper aspect of the device. The occlusion device is in a deployed configuration with arrows showing blood flow through the device with the release slits 1675 closed. The slits 1675 may be formed by cutting the covering 1600. The lengths of the slits may vary depending upon the configuration of an occlusion device. In some embodiments, the slits 1675 range from 5 mm to 10 mm in length. In some embodiments, the slits correspond to the adjacent cell 1517. In some embodiment, the slits 1675 are formed only in the unattached covering portion 1685. In optional embodiments, slits 1675 may be formed in any of the distal attached, proximal attached or the unattached zones 1680, 1690, 1685.

FIG. 40B is a perspective view of the occlusion device of FIG. 40A with an outer sheath 1680 moving over the proximal end of the device. The movement of the outer sheath 1680 prevents flow out of the proximal end of the device causing blood to urge open and flow through the slits 1675. This relief of blood flow and pressure within the occlusion device assists in the advancement of the outer sheath and the transition to the stowed configuration of the occlusion device.

FIG. 40C is a perspective view of an occlusion device 1500 having a pressure release feature 1687 within the scaffold covering 1600 along an upper aspect of the device. The pressure release feature 1687 is located under a flexible cover 1688. The flexible cover 1688 is attached to the outer layer of the cover 1600.

FIG. 40D is a perspective view of the occlusion device of FIG. 40C showing the operation of the flexible cover 1688 and the pressure release feature 1687. Movement of an outer sheath 1580 as shown in FIG. 40B will produce the flow relief mode of FIG. 40D. The flexible cover 1688 is shown lifted away from the outer surface of the occlusion device 1500 allowing flow through a relief feature 1687 in an upper portion of the occlusion device. The relief feature 1687 is illustrated with a diamond shape and an approximate length of 5-10 mm. More than one relief feature 1687 may be provided in additional embodiments. The cover 1688 can also be attached in a different location or more than one location(s).

FIG. 40E is a perspective view of an occlusion device having a series of pressure release features 1687 such as in FIG. 40D. The relief features 1687 are positioned along an upper aspect of the device. Movement of an outer sheath 1580 as in FIG. 40B will produce flow through the relief features 1687. More or fewer or different sized and shaped relief features may be provided such as circle shaped or oval shaped. The dimensions of the relief features may have a long dimension ranging from 5 mm to 10 mm. The relief features 1687 may be formed as described in FIG. 40A by cutting, stamping or punching the cover material to form the relief feature.

FIG. 40F is a perspective view of an occlusion device having a pressure release feature provided by the tapered shape of the cover 1600. The cover is wider along the inferior aspect 1636 than along the upper aspect 1633. The result is that there more covered scaffold along the bottom portion along 1636 and less covered scaffold along the upper portion 1633 of the device. The shape of the tapered cover is selected to correspond with the probable locations of the one or more branch vessels to be reversibly occluded by the occlusion with perfusion device. Advantageously, the movement of an outer sheath as in FIG. 40B will produce flow adjacent to the upper portion 1633 via the open scaffold 1513.

FIG. 41A is a perspective view of the vascular occlusion device of FIG. 38 after the slider is moved into the proximal position to fully transition the covered scaffold into the deployed configuration. The slider on the handle is in a proximal position with the outer shaft or sheath withdrawn from the covered scaffold which is shown in a deployed configuration.

FIG. 41B is a perspective view of the vascular occlusion device of FIG. 41A with a section of the outer shaft removed to position the deployed covered scaffold adjacent the handle with the slider shown in the proximal position to fully transition the covered scaffold into the deployed configuration as shown.

FIG. 41B also shows a side view of the handle 1550 with the slider knob or slider 1556 in a proximal position to withdraw the outer shaft and allow the scaffold structure to be in a deployed configuration as shown in FIG. 41B. The handle 1550 includes an upper handle housing 1552 and a lower handle housing 1554. The hemostasis valve 1599 is also visible in this view.

FIG. 42 is an exploded view of the handle embodiment of FIGS. 41A and 41B. A slider 1556 goes over tab 1558 on slider rack 1560. There is a slot 1553 in upper handle housing 1552 allows proximal and distal translation of slider 1556 (see FIG. 43). The slider rack 1560 has a tab 1558 used to engage with slider 1556 through slot 1553. The slider rack teeth 1562 are arranged to engage with inner gear 1579 on double gear pinion 1575. The outer shaft rack 1570 includes outer shaft rack teeth 1572. There is a receiver 1585 for engaging with outer shaft coupler 1586 on outer shaft 1580. Double gear pinion 1575 includes outer diameter teeth 1577 to engage with outer shaft rack teeth 1572 of outer shaft rack 1570. The double gear pinion includes inner diameter teeth 1579 to engage with the slider rack teeth 1562 of slider rack 1560. The outer shaft 1580 has a proximal end 1582 and a distal end 1584. The outer shaft coupler 1586 is adjacent to the outer shaft proximal end 1582 within the handle 1550. The double gear pinion and other components of the handle may be configured to provide a 3:1 gear ratio for transmitting the movement of the slide 1556 into translation of the outer sheath 1580.

FIG. 43 is a cross section view of the handle embodiment of FIG. 41B. The tab 1558 is shown within the slider 1556 which is positioned in the proximal position within slot 1553. The spaced apart position of the receiver 1585 and the outer shaft coupler 1586 relative to the distal end of handle 1550 is also shown in this view. The outer shaft rack teeth 1572 are shown engaged with the outer diameter teeth 1579 of double gear pinion 1575.

In various embodiments, the occlusion system describe herein is compatible with other cardiac catheterization lab or interventional radiology lab workflow, designed with user-friendly functions and inserted and removed from patient similar to insertion of off-the-shelf introducer sheath with add-on function of temporary peripheral vascular occlusion. The device is an “assist device” which does not interfere with the standard catheterization procedure and comply with the standard activities in the catheterization lab.

FIG. 44A is a cross section of a vascular occlusion device positioned for occlusion of the renal arteries and perfusion of the arterial tree in the lower extremities. This figure illustrates the distention or bulging 1645 of an unattached portion 1685 of the scaffold covering 1600 in response to the blood flow pressure generated within the scaffold 1510. As seen in this view, the unattached section 1685 of the scaffold covering is partially distended 1645 into and further ensuring the desired occlusion of the peripheral artery. In this illustrative embodiment, the temporarily occluded vessels are the renal arteries. Here, a portion of scaffold covering has bulged 1645 into and further occludes the renal artery ostia (see for example step 4640 in method 4600 or step 4740 in method 4700). While illustrated for use with the renal ostia, the position of the unattached zone 1685 relative to the scaffold 1510 as well as the amount or size of the unattached portion 1685 may be adapted based on the use of the vascular occlusion device 1500 when used with any of a wide array of peripheral structures while also allowing for perfusion flow beyond the temporarily occluded portion of the vasculature. Other exemplary peripheral vasculatures which may be additionally at least partially occluded using the bulging response 1645 of the unattached scaffold covering zone 1685 include, for example, a hepatic artery, a gastric artery, a celiac trunk, a splenic artery, an adrenal artery, a renal artery, a superior mesenteric artery, an ileocolic artery, a gonadal artery and an inferior mesenteric artery while simultaneously allowing perfusion flow through or around the at least partially covered scaffold structure to distal vessels and structures.

FIG. 44B is an alternative of the embodiment of FIG. 29A-C within a portion of an aorta adjacent to a pair of openings to branch vessels. The cover 1600 is attached only at the proximal and distal end attachment zones 1690, 1680 at the proximal and distal ends of the device 1515, 1513. Portions of the cover 1600 that are unattached to the scaffold move in response to blood flow through the device. The unattached cover portion 1685 is shown against the scaffold 1510 in FIG. 44B.

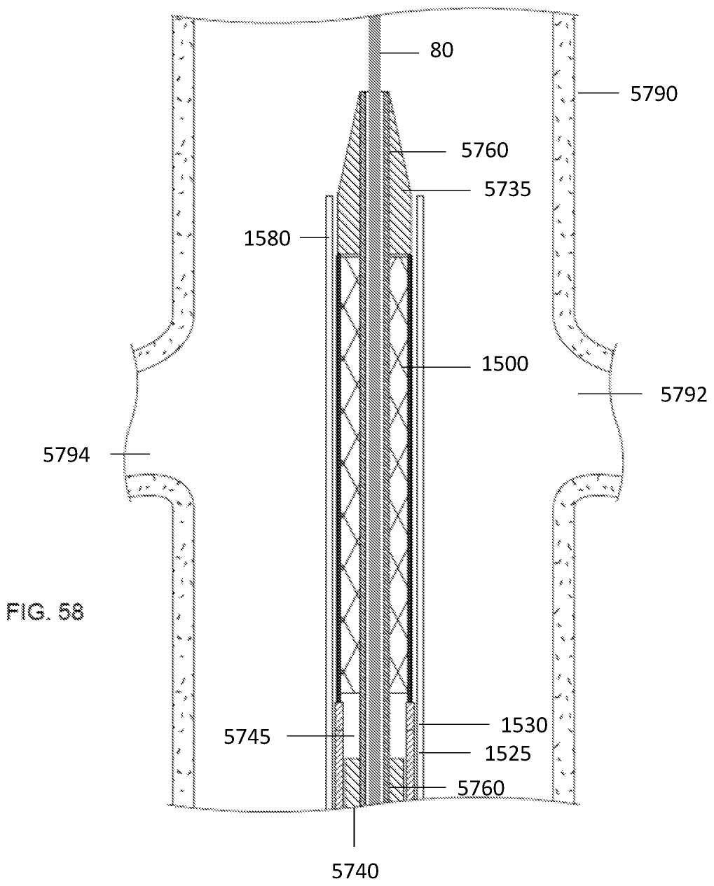

FIG. 44C is a view of the device of FIG. 44B showing how unattached portions 1685 of the cover 1600 have deflected away from the scaffold 1510 and at least partially occluded the branch vessels of the aorta. In various embodiments of the occlusion with perfusion device whether used alone or in combination as a single point vascular access device, the covering 1600 may be placed with a variety of different configurations relative to both the type of protection desired, the number of uncovered, covered and attached, covered and attached zones or other combinations as may be advantageously employed for the various different therapeutic lengths described with regard to FIG. 70. Still further, some embodiments of an occlusion with perfusion device employed for multiple branch vessels or those uses within therapeutic lengths 2 and 3 are adapted and configured for insertion and positioning in the vasculature using an embodiment of a modified dilator with pocket as illustrated and described in FIG. 58.

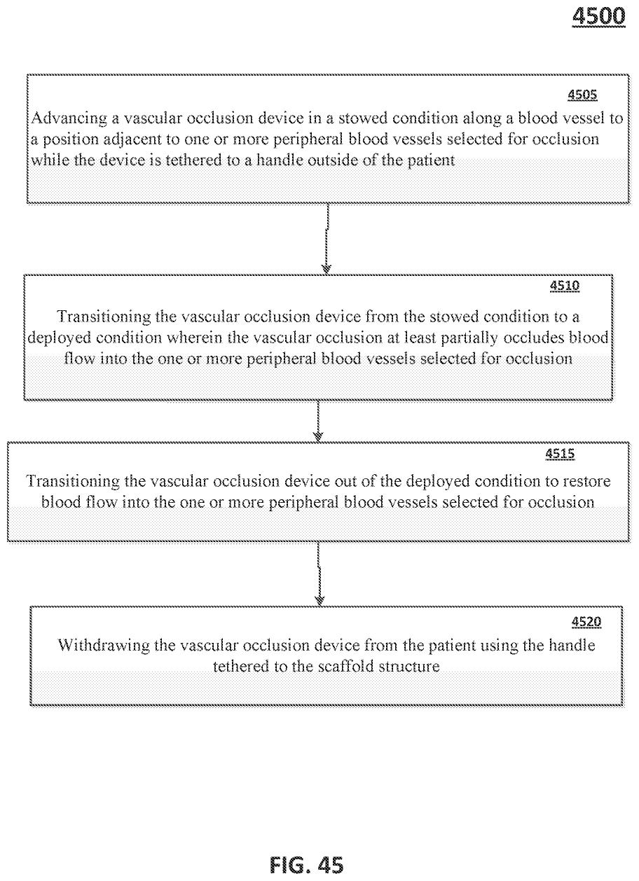

FIG. 45 is a flow chart of an exemplary method of providing occlusion with perfusion using an embodiment of a vascular occlusion device according to the method 4500.

First, at step 4505, there is the step of advancing a vascular occlusion device in a stowed condition along a blood vessel to a position adjacent to one or more peripheral blood vessels selected for occlusion while the device is tethered to a handle outside of the patient.

Next, at step 4510, there is the step of transitioning the vascular occlusion device from the stowed condition to a deployed condition wherein the vascular occlusion at least partially occludes blood flow into the one or more peripheral blood vessels selected for occlusion.

Next, at step 4515, there is the step of transitioning the vascular occlusion device out of the deployed condition to restore blood flow into the one or more peripheral blood vessels selected for occlusion.

Finally, at step 4520, there is the step of withdrawing the vascular occlusion device from the patient using the handle tethered to the scaffold structure.

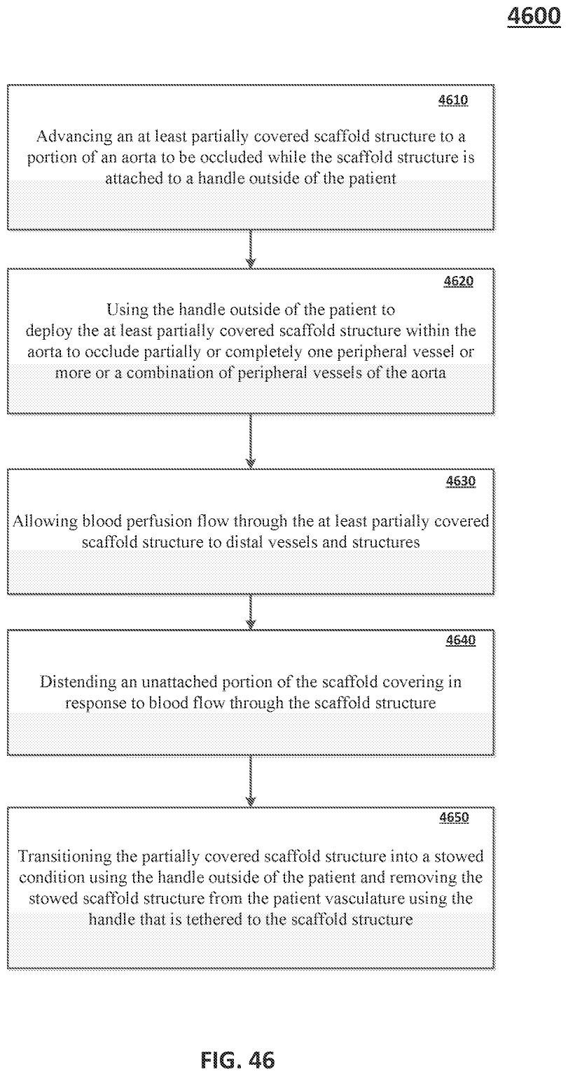

FIG. 46 is a flow chart of an exemplary method of providing occlusion with perfusion using an embodiment of a vascular occlusion device according to the method 4600.

First, at step 4610, there is the step of advancing an at least partially covered scaffold structure to a portion of an aorta to be occluded while the scaffold structure is attached to a handle outside of the patient.

Next, at step 4620, there is the step of using the handle outside of the patient to deploy the at least partially covered scaffold structure within the aorta to occlude partially or completely one peripheral vessel or more than one or a combination of peripheral vessels of the aorta. This step may be appreciated with reference to FIG. 70.

Next, at step 4630, there is the step of allowing blood perfusion flow through the at least partially covered scaffold structure to distal vessels and structures.

Next, at step 4640, there is a step of distending an unattached portion of the scaffold covering in response to blood flow through the scaffold structure.

Next, at step 4650, there is a step of transitioning the partially covered scaffold structure into a stowed condition using the handle outside of the patient. Thereafter, removing the stowed scaffold structure from the patient vasculature using the handle that is tethered to the scaffold structure.

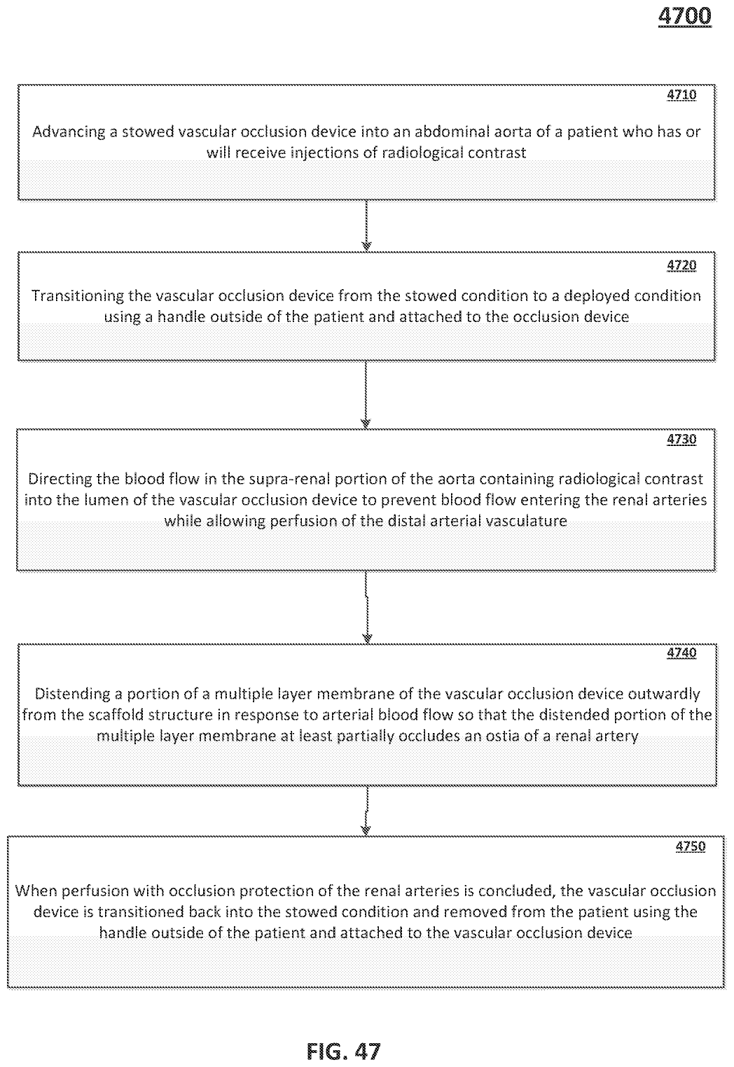

FIG. 47 is a flow chart of an exemplary method of providing occlusion with perfusion using an embodiment of a vascular occlusion device according to the method 4700.

First, at step 4710 there is a step of advancing a stowed vascular occlusion device into an abdominal aorta of a patient who has or will receive injections of radiological contrast.

Next, at step 4720, there is a step of transitioning the vascular occlusion device from the stowed condition to a deployed condition using a handle outside of the patient and attached to the occlusion device.

Next, at step 4730, there is a step of directing the blood flow in the supra-renal portion of the aorta containing radiological contrast into the lumen of the vascular occlusion device to prevent blood flow entering the renal arteries while allowing perfusion of the distal arterial vasculature.

Next, at step 4740, there is a step of distending a portion of a multiple layer membrane of the vascular occlusion device outwardly from the scaffold structure in response to arterial blood flow so that the distended portion of the multiple layer membrane at least partially occludes an ostia of a renal artery.

Next, at step 4750, there is a step performed when perfusion with occlusion protection of the renal arteries is concluded. At this point, the vascular occlusion device is transitioned back into the stowed condition and removed from the patient using the handle outside of the patient and attached to the vascular occlusion device.

FIG. 48 is a side view of an exemplary covered scaffold according to one embodiment of the vascular occlusion device. The covered scaffold indicates the distal attachment zone 1680, the proximal attachment zone 1690 and the unattached zone 1685 that indicate whether a portion of the scaffold covering 1600 is joined to the scaffold structure 1510 in that zone. The advantageous placement of the unattached zone 1685 allows embodiments of the covered scaffold to have a portion of the scaffold covering 1600 bulge or distend in response to blood flow. The distended scaffold covering 1600 may further occlude an adjacent peripheral vessel opening providing additional and targeted occlusion capabilities.

In some embodiments, the scaffold covering 1600 comprises a multiple layer structure that is attached to all or to select portions of the scaffold frame 1510. In some embodiments, the multiple layer covering is used to encapsulate all or a portion of the scaffold structure including the legs. The multiple layer scaffold covering may be a partial scaffold covering as seen in the embodiments of FIGS. 27, 28B, 30, 31, 32, 34, 35, 36 and 37 in relation to the percentage of the scaffold that is covered along the central axis 1511 or tapered in relation to the longitudinal axis as in FIG. 34. In one embodiment the scaffold distal end 1620 may include a distal folded portion 1622 over the distal end of the scaffold 1515. Along the same lines, the scaffold proximal end 1630 may include a proximal folded portion 1632 over the proximal end of the scaffold 1513, optionally including covering the legs 1519 and optionally including covering the connection tabs 1521. (See FIGS. 29A, 29B and 29C).

FIG. 49 is a partial exploded view of a portion of each of the individual layers that together form a multiple layer scaffold covering embodiment. Each one of the layers is shown with an arrow indicating an orientation of a characteristic or quality of that layer. Illustrated orientations are provided relative to the central axis of the scaffold structure as parallel (a), transverse (b) or oblique (c) or (d). In one embodiment, the orientation of each layer of the multi-layer structure determined by the predominant orientation of node and fibril microstructures within the layer as indicated by the arrows in FIG. 49. Additional details of adaptation of this characteristic of the multiple layer scaffold covering may be appreciated by reference to U.S. Pat. No. 8,840,824, incorporated herein by reference for all purposes. In still further embodiments, these or other characteristics of each of the layers of the multiple layer scaffold covering may be selected and positioned in the stack to further adapt characteristics such as strength, flexibility or permeability, as desired for a specific performance in an application of the vascular occlusion device.

In still other embodiments, any of the above described disturbing means such as a tunnel membrane illustrated and described in FIGS. 12A-13D may be covered using an embodiment of the scaffold covering 1600 including a multiple layer embodiment as well as the inclusion of the proximal and distal attachment zones and an unattached zone as described above. In still other embodiments, the embodiments shown of the occlusion with perfusion devices shown in FIGS. 19A-22B of US Patent Application Publication US 2018/0250015 may be modified to also include the scaffold covering and attachment and unattached zones described herein. It is to be appreciated that one or more of the layers of the multiple layers used to form the multiple layer embodiments of the scaffold layer 1600 may be selected from any of a wide array of suitable biocompatible materials including biocompatible soft or semi-soft plastics. The tunnel membrane previously described or the scaffold covering 1600 may comprise multiple individual layers of the covering material where one or more of the layers may differ from other layers. Additionally or optionally, the orientation of one or more layers used to form the scaffold covering may be selected so that in the aggregate multiple layer scaffold covering a desired characteristic or property of the scaffold covering, the covered scaffold, or the vascular device may better form the desired degree of occlusion with perfusion. In some embodiments one or more of the layers of a multiple layer scaffold covering 1600 is selected from one or more flexible films, ribbons, membranes such as polytetrafluoroethene (PTFE), fluorinated ethylene propylene (FEP), copolymers of hexafluoropropylene and tetrafluoroethylene, perfluoroalkoxy polymer resin (PFA), expanded polytetrafluoroethylene, silicone rubber, polyurethane, PET (polyethylene terephthalate), polyethylene, polyether ether ketone (PEEK), polyether block amide (PEBA), or other materials suited to the performance characteristics of the scaffold covering. In still other advantageous combinations of multiple layers of a scaffold covering, the layers used in the scaffold covering are selected to enhance the billowing or bulging response of an unattached zone in response to pressure waves within the blood flow. The billowing or bulging response may be modified based on the occlusion characteristics needed for selected peripheral vasculature where embodiments of the vascular occlusion devices with distal perfusion may be employed.

In view of the above, in other additional optional embodiments and configurations of the vascular occlusion devices described herein, an embodiment of a vascular occlusion device may be used to provide a method of providing occlusion of a portion of the vasculature of a patient with perfusion distal to the occlusion portion using the following method. First, there is a step of advancing a vascular occlusion device in a stowed condition along a blood vessel to a position adjacent to one or more peripheral blood vessels in the portion of the vasculature of the patient selected for occlusion while the vascular occlusion device is tethered to a handle outside of the patient. Next, there is a step of transitioning the vascular occlusion device from the stowed condition to a deployed condition using the handle wherein the vascular occlusion device at least partially occludes blood flow into the one or more peripheral blood vessels selected for occlusion. Next, the position of the vascular occlusion device which engages with the superior aspect of the vasculature to ensure that blood flow is directed into and along the lumen defined by the covered scaffold structure. As a result, the scaffold structure occludes the vessels targeted for temporary occlusion while directing the blood flow along the lumen of the vascular occlusion device through the interior of the covered scaffold to thereby maintain blood flow to blood vessels distal to the occluded portion of the vasculature. Furthermore, in some embodiments, the unattached zone of the covered scaffold deflects, bulges, or deforms in response to the blood flow now directed through the lumen of the covered scaffold. As a result, a portion of the unattached zone of the covered scaffold is urged into an adjacent opening of the peripheral blood vessel that is the target of the selected temporary occlusion procedure. It is to be appreciated that the location, size and number of unattached zones of a covered scaffold embodiment may vary according to the size, number and location of peripheral vessels selected for temporary occlusion. Thereafter, when the period of providing temporary occlusion is completed, the step of transitioning the vascular occlusion device from the deployed condition to the stowed condition using the slider on the handle which remains connected to the scaffold structure at all times during use. Once in the stowed configuration, the step of withdrawing the vascular occlusion device from the patient is performed by appropriate movement of the handle.

In another aspect, a method for mitigating exposure of the kidneys to medical contrast media is disclosed. The method comprises: inserting a catheter having a partially covered scaffold device into the vasculature and advancing into a desired position within an abdominal aorta; and deploying the scaffold so that the covering, membrane or tunnel structure is in a position to partially or complete occlude the renal arteries during use of contrast media while simultaneously providing perfusion blood flood distal to the occluding device. In certain embodiments, the insertion of the partially covered scaffold occlusion device to an aorta is accomplished by a transfemoral artery approach or by a trans-branchial artery approach or by a trans-radial artery approach. In some embodiments, the catheter and scaffold occlusion device are inserted along a guidewire and moved into a position to partially or completely occlude one or more blood vessels under appropriate medical imaging guidance such as fluoroscopy. Additional details and illustrations of the various vascular access routes described herein may be appreciated with reference to US Patent Application Publication US 2013/0281850 entitled, “Method For Diagnosis and Treatment of Artery,” which is incorporated herein by reference for all purposes. The above details and alternative method steps may also be applied to provide additional embodiments and variations to the steps detailed for methods 4500, 4600 and 4700 described herein.

Those of ordinary skill will appreciate that the devices and methods described herein meet the objective of a catheter based vascular occlusion system that will be able to be used to access the aorta with the ability to provide temporary occlusion of target vasculature while maintaining perfusion to the lower limbs vasculature. US Patent Application Publication US 2016/0375230 and US 2018/0250015 are incorporated herein by reference for all purposes.

The various embodiments of the vascular occlusion with perfusion devices described herein provide in a general way a flow disturbing means within the blood flow of the aorta. The distal most end of the scaffold engages substantially circumferentially with the interior wall of the aorta so that substantially all of the blood flow in the aorta flows into and along the central axis of the scaffold and out of the scaffold proximal openings. In one illustrative embodiment, a vascular occlusion device is positioned such that the scaffold or tunnel membrane shunts blood flowing from the supra-renal aorta through the scaffold or tunnel membrane, bypassing the renal arteries, and into the intra-renal aorta as the flow exits the scaffold. Alternative distal most segments of the scaffold may be used for greater contact area with the blood vessel where the vascular occlusion with perfusion device is employed. Optionally, the distal most segment of the scaffold may be in the shape of a flared distal end of the scaffold (see FIGS. 40 and 41). In an additional alternative embodiment, a flat distal engagement segment such as that exemplified by segment 1811 in FIG. 13A may also be used. Additionally or optionally, one or more flared segments, or one or more flat segments may be used alone or in combination to ensure fluid tight contact the wall of the supra-renal aorta and the wall of the infra-renal aorta, respectively if desired. Similar modifications may be made for use in other combinations of occlusion with perfusion on other possible peripheral vessels for clinical scenarios beyond protection of the kidneys from exposure to contrast agents. The aperture 106 may be substantially the same as the aperture 207 described previously herein. Regardless of the vascular occlusion embodiment selected, the shunting period or period of time that occlusion with perfusion is utilized may be (a) synchronized with the injection of a contrast media by a physician or (b) used so long as the occlusion of the selected peripheral vessel is clinically necessary however irrespective of length of use the scaffold remains attached to the handle which is outside of the patient's vasculature. In other words, the vascular occlusion devices that provide selective occlusion with perfusion are temporary vascular devices that are always tethered outside of the body during use. Still further, it is to be appreciated that the occlusion or shunting period should be kept to a minimum amount of time to shunt the contrast media but not long enough to cause renal ischemia by preventing blood flow to the kidneys. The kidneys are resistant to transient ischemia, therefore the shunting period may be tuned to avoid ischemia, depending upon the specific clinical situation where the device is being employed.

Exemplary Vascular Occlusion Devices and Covered Scaffolds

In some specific embodiments, the scaffold 1510 is fabricated as a laser cut tube of overall length from the connection tab 1521 on the legs 1519 to the scaffold distal end 1515 ranges from 40 mm to about 100 mm. Typically, the vascular occlusion device is delivered and maintained within a stowed configuration compressed with an 8 Fr compatible outer shaft or sheath. As best seen in FIG. 39A, the outer diameter of the outer sheath ranges from outer shaft overall diameter is between 0.100 inches and 0.104 inches. When the outer shaft is withdrawn as shown in FIG. 39C, the deployed condition of the covered scaffold structure into the vasculature, such as the lower aorta, has a deployed diameter ranging from 15 mm to 35 mm or an outer diameter ranging from 19 mm to 35 mm. As detailed in FIGS. 48 and 49, the scaffold covering may be formed from multiple layers of material to a final thickness of 0.001 inches in an unattached zone 1685 and 0.002 inches in each of the distal attached zone 1680 and the proximal attached zone 1690. Additionally, in other embodiments the vascular occlusion device may be characterized by the occlusive length of the deployed covered scaffold structure. The occlusive length of a covered scaffold structure is measured from the scaffold distal end 1515 to the distal end of the scaffold transition zone 1518 where the scaffold transitions to two or three or fewer legs and attachment to the inner shaft. In various embodiments the covered scaffold has an occlusive length ranging from 40 mm to 100 mm. In some embodiments, the vascular occlusion device has a 65 cm working length as measured from the handle 1550 to the distal end of the inner shaft 1528 and the atraumatic tip 1532.

Turning now to an exemplary bare scaffold structure as shown in FIG. 18. The scaffold cell geometry is laser cut into a tube and electropolished to a smooth finish. The resulting thickness of the scaffold is about 0.008″. There are typically 3 to 6 cells arranged along the longitudinal axis and 6 to 12 cells arranged along the perimeter. In general, a typical cell opening ranges from 1 cm to 2 cm along the longitudinal axis and from 0.5 cm to 1.5 cm along the circumference. In some embodiments, the cell orientation may be approximately diamond shaped when deployed with a major axis along the longitudinal axis of the scaffold and device in the range of 4 cm to 6 cm and a minor axis along the circumference of the device that ranges from 25 mm to 100 mm.

Additional details of an exemplary occlusion with perfusion devices and systems are available with reference to co-pending International Application No. PCT/US2020/052899 titled “Devices and Methods for at least Partially Occluding A Blood Vessel While Maintaining Distal Perfusion,” filed on Sep. 25, 2020, U.S. Pat. No. 10,300,252 to Lee et al., and U.S. Pat. No. 10,441,291 to Koo et al. Additional details of introducer devices are available with reference to U.S. Pat. No. 6,090,072 to Kratoska et al, U.S. Pat. No. 5,542,936 to Razi, US Patent Application Publication US 2015/0094795 to Ginn, et al., and US Patent Application Publication US 2013/0281850 to Okajima et al.

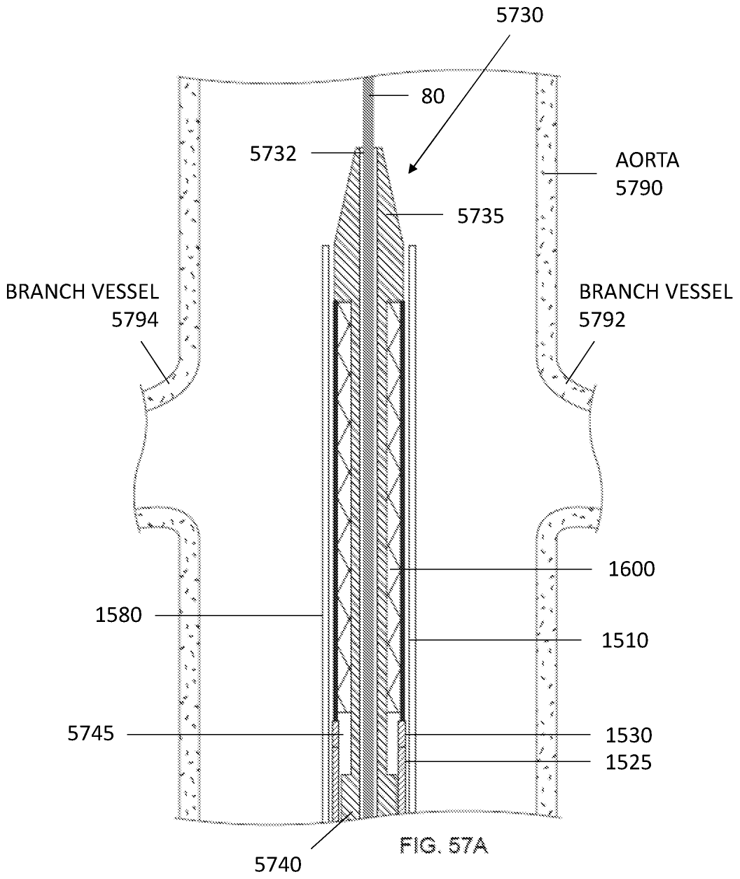

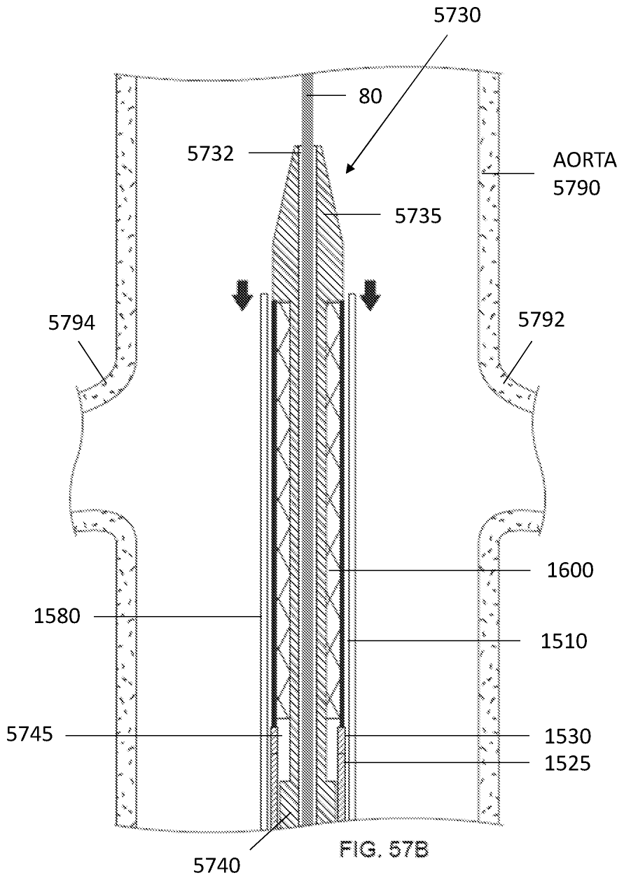

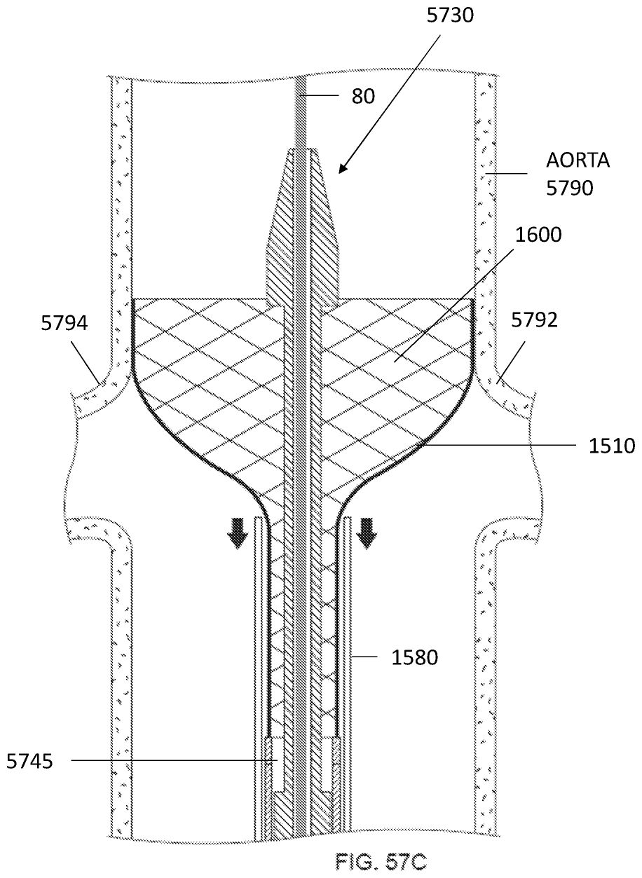

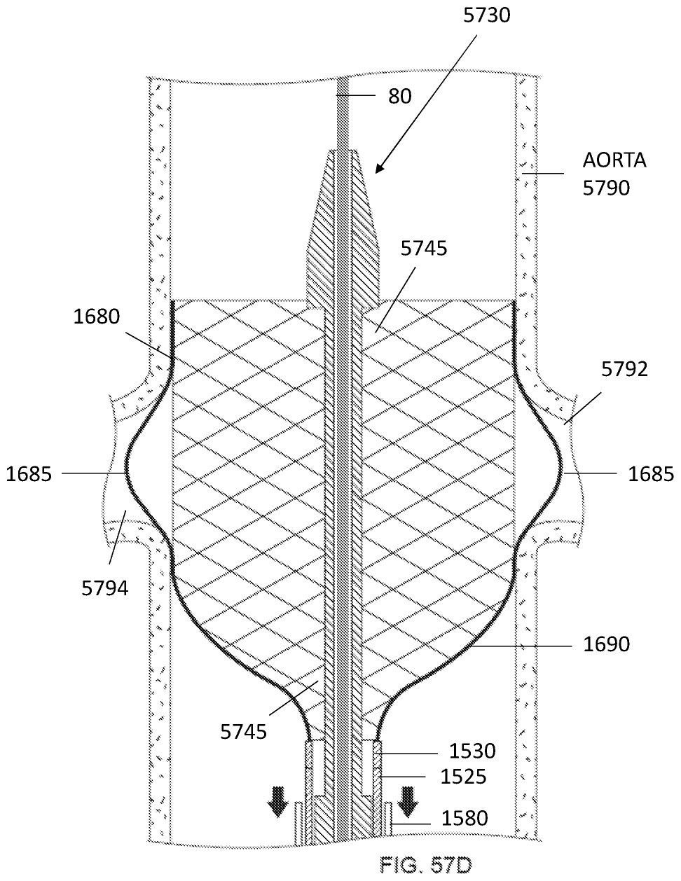

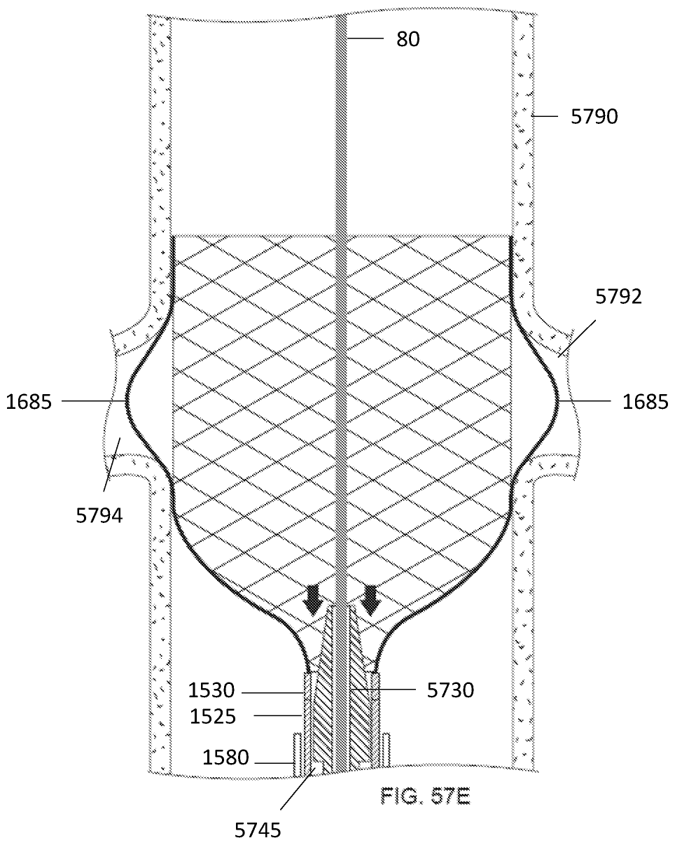

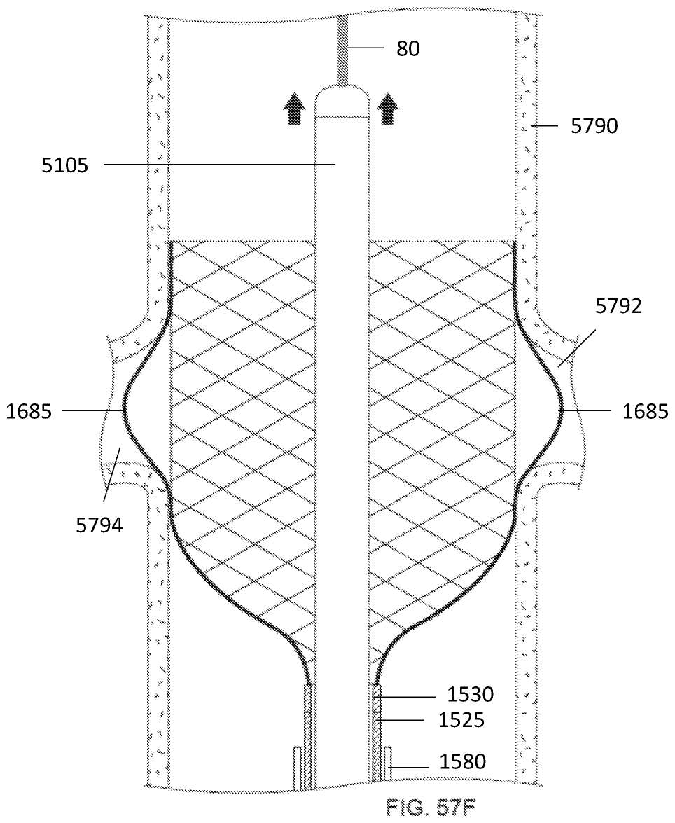

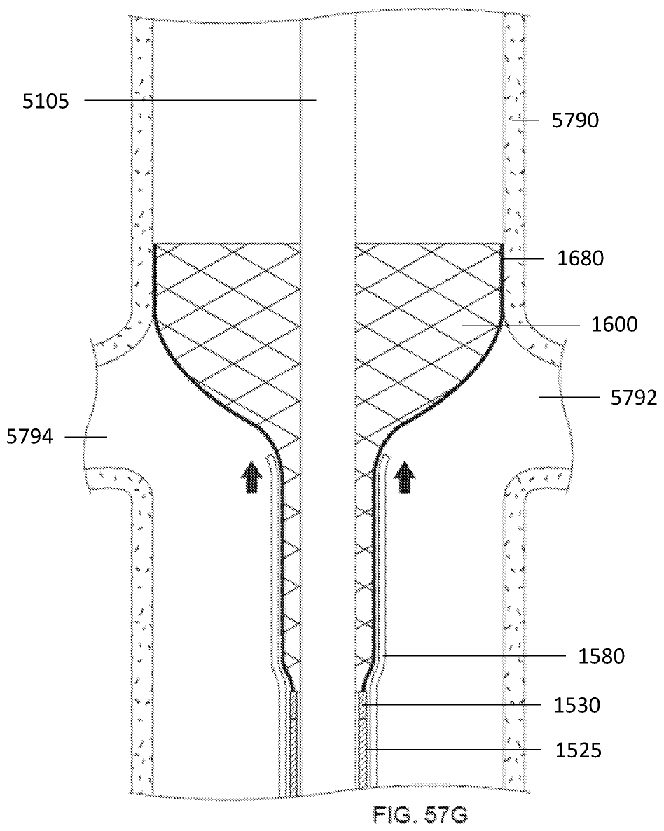

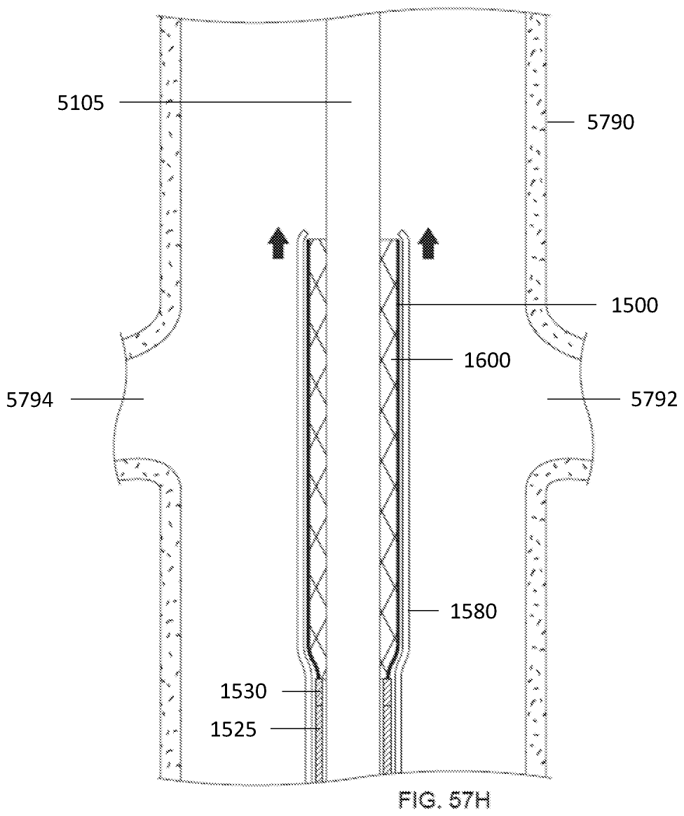

The various embodiments of introducer with occlusion with perfusion devices described herein are used using conventional surgical techniques for introducer sheath and dilator kits. Introducers may be modified to function as an outer shaft or expandable outer shaft as described herein. Similarly, unmodified dilators may be used with an occlusion with perfusion device as described herein. Advantageously, the overall dimensions of the combination devices described herein may be reduced by modifying a dilator to have a pocket sized to receive a stowed occlusion with perfusion device. Modified dilators having pockets for device stowage are further described with respect to FIGS. 57B, 58 and 59.

In still other embodiments, there may also be an elongate dilator with a proximal Luer assembly configured to be inserted into the working lumen of the introducer sheath through a proximal seal coupled to a hub structure, which is also coupled to an extension tube with stopcock, which may be utilized for infusion of fluids into the introducer lumen, for example. The inventive introducer will comprise an elongate tubular member coupled proximally to the hub and being made from a relatively non-expandable polymeric material or combination of polymeric materials, or other material based on whether or to what degree the introducer will be adapted and configured to have expansion capabilities.

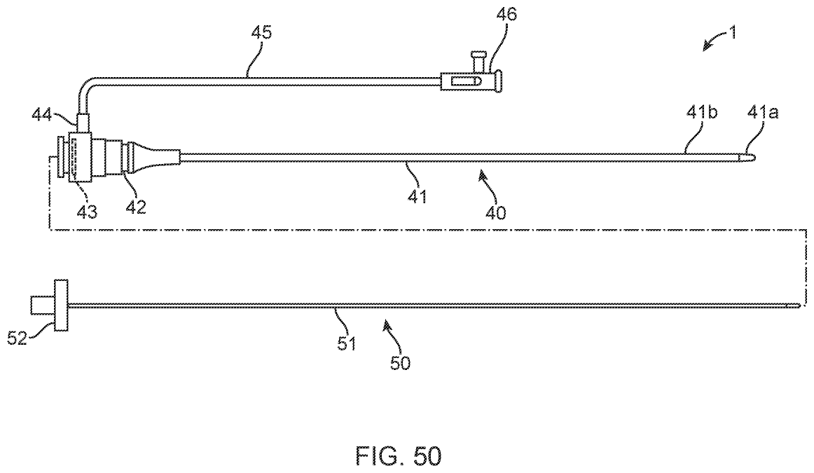

FIG. 50 is a plan view of an introducer assembly without the occlusion with perfusion device attached by way of example.

As shown in FIG. 50, the introducer assembly 1 includes the introducer sheath 40 for securing an access route to the inner of a body lumen, and a dilator 50 for assisting the insertion of the introducer sheath 40 that is to be percutaneously indwelled in the body lumen.

The introducer sheath 40 includes a sheath having open distal and proximal ends. More specifically, the introducer sheath 40 includes, for example, a sheath tube 41 having open distal and proximal ends, a sheath hub 42, a hemostasis valve 43, a side port 44, a tube 45, and a three-way cock 46. The sheath tube 41 is percutaneously put indwelling (indwelled) in a body lumen, after which an angiography catheter, serving as an example of a diagnostic instrument, or a balloon, a stent or the like, serving as an example of a therapeutic instrument, is inserted into and moved along the sheath tube 41, to be thereby introduced into the body lumen. The sheath hub 42 permits the sheath tube 41 and the side port 44 to communicate with each other interiorly of the sheath tube 41 and the side port 44. The hemostasis valve 43 is incorporated in the sheath hub 42. The hemostasis valve 43 stanches (stops) blood flowing out of a blood vessel through the sheath tube 41. The side port 44 permits communication between the sheath tube 41 and the tube 45. The tube 45 permits communication between the side port 44 and the three-way stopcock 46. The three-way stopcock 46 is used to inject a liquid such as physiological saline into the introducer sheath 40 through the tube 45 and the side port 44.

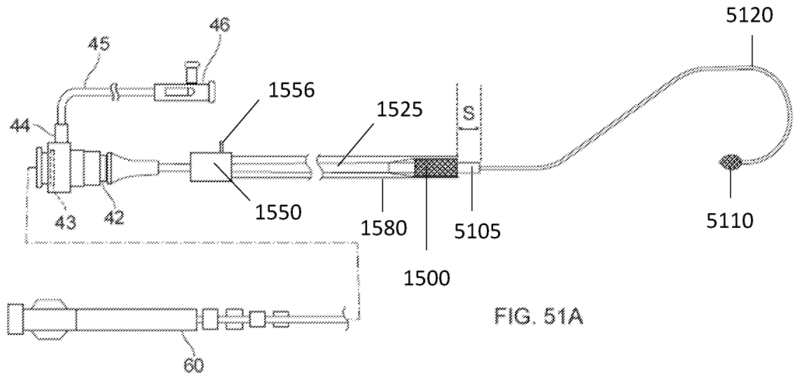

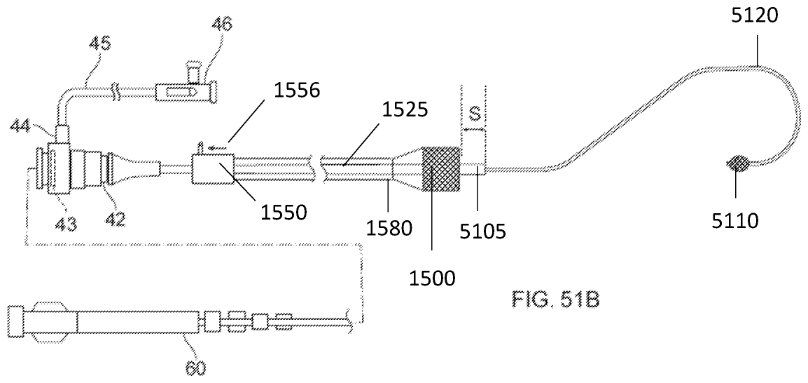

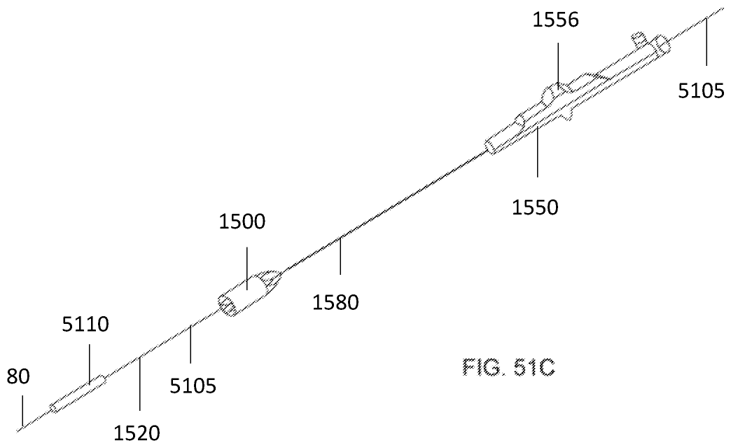

It is to be appreciated that in the various embodiments that follow, an introducer sheath 40 or similar component may be adapted for use as an outer shaft or outer sheath as detailed herein. Similarly, the various operable capabilities described above or with respect to FIGS. 50, 51A and 51B may be provided by any of the occlusive devices or handle designs such as in FIGS. 25A, 30-38, 41A, 41B, 42, 43, 44A, 51C, 55, 60, 68A, 68C, 68D

Examples of the material forming the outer shaft or sheath 40 include polyethylene, polyethylene terephthalate, polypropylene, polyamides, polyamide elastomers, polyimides, polyurethane, PEEK (polyether ether ketone), and fluorine-based polymer such as ETFE, PFA, or FEP, among which ETFE and PEEK are preferred in consideration of an anti-kinking effect which will be described later.

The dilator 50 includes, for example, a dilator tube 51 and a dilator hub 52. The dilator tube 51 of the dilator 50 is inserted into and moved along the sheath tube 41 so the distal end of the dilator is positioned distally beyond the distal and of the sheath. The dilator tube 51 (dilator) assists the insertion of the introducer sheath 40 which is to be percutaneously indwelled in a body lumen. The dilator hub 52 holds the dilator tube 51 in the state of being detachably attachable to the sheath hub 42. The outer diameter of the dilator tube 51 is substantially equal to or slightly smaller than the inner diameter of the sheath tube 41. The various dilator embodiments described in FIGS. 57A, 58 and 59 may also include these characteristics and capabilities in addition to the modifications described herein.

FIG. 51A is a plan view showing a condition in which a diagnostic instrument or a therapeutic instrument 5110 at the distal end of catheter 5120 and controlled or manipulated by an appropriately configured handle 60. The catheter 5120 has been inserted through the lumen of the guide catheter 5105. The guide catheter 5105 is inserted in the introducer sheath occlusion with perfusion device combination. The diagnostic instrument or the therapeutic instrument 5110 is accessing the vasculature via the guide catheter 5105 within the lumen of the inner shaft 1525 via handle 1550. The inner shaft 1525 is coupled to the occlusion with perfusion device 1500 as described herein. The occlusion with perfusion device 1500 is in a stowed condition against the outer wall of the guide catheter 5105. The guide catheter 5105 extends a distance “S” beyond the distal end of the outer sheath 1580 and the occlusion with perfusion device 1500. FIG. 51A illustrates a configuration or assembly in which a diagnostic instrument or a therapeutic instrument 5110 is inserted in the outer or introducer sheath 40 according to this embodiment wherein the inner diameter of the outer sheath or introducer 1580 is sufficient to retrieve the perfusion with occlusion device 1500 between an inner wall of the outer sheath 1580 and an outer wall of the guide catheter 5105.

FIGS. 51A and 51B illustrate the condition in which an instrument 60 composed of a diagnostic instrument or a therapeutic instrument 5110 is inserted in the introducer sheath and occlusion with perfusion device combination.

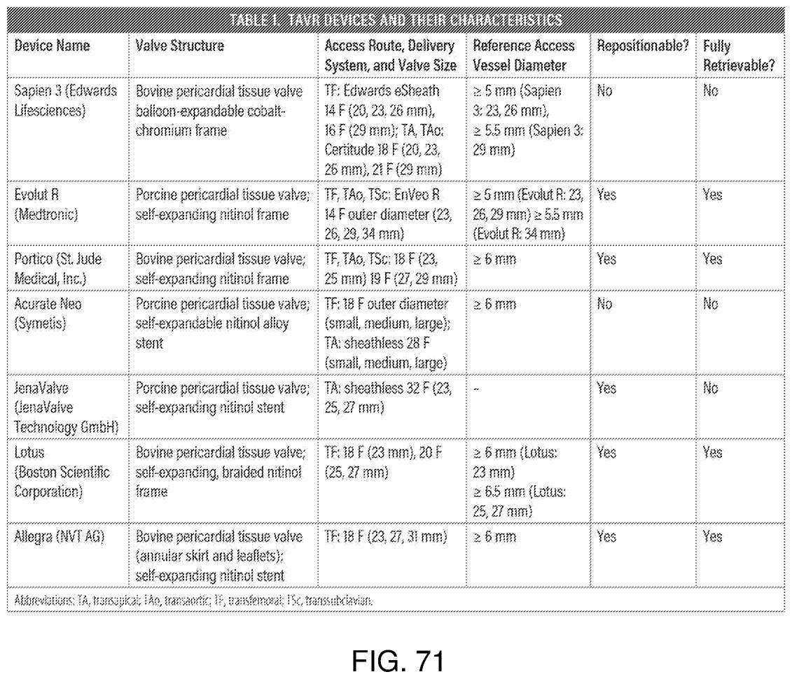

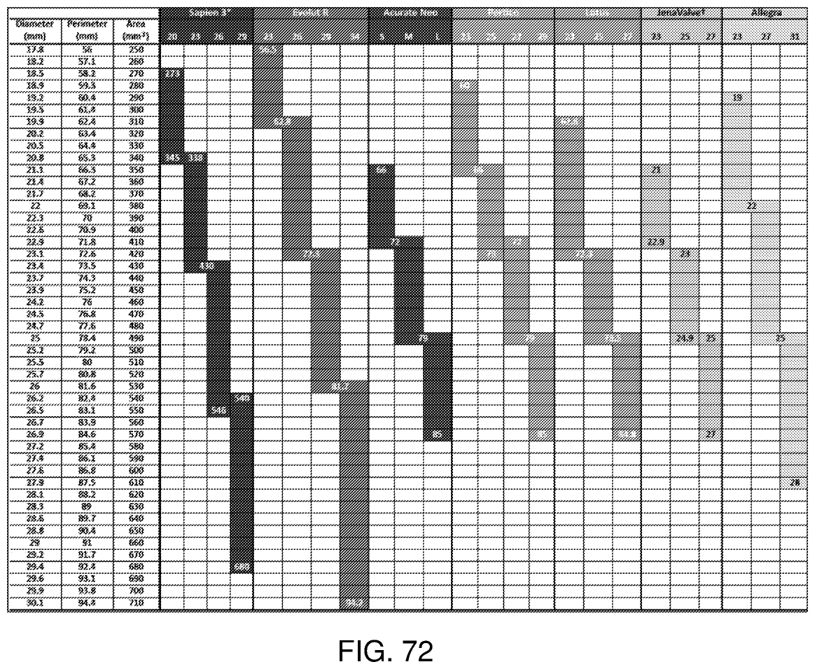

The instrument 60 is inserted into the introducer sheath 40 after the introducer sheath 40 is inserted in a blood vessel and after the dilator 50 is drawn out of the introducer sheath 40 and the guide catheter 5105 introduced and advanced beyond the sheath 40 and the occlusion device 1500. The instrument 60 has an elongated body, and is inserted into the blood vessel through the introducer sheath 40. In the case of the instrument 60 being a diagnostic instrument, examples of the instrument 60 include an angiography catheter, an intravascular ultrasound testing instrument, or an intravascular optical coherence tomography instrument. In the case of the instrument 60 being a therapeutic instrument, examples of the instrument 60 include a balloon catheter, a drug-eluting balloon catheter, a bare metal stent, a drug-eluting stent, a drug-eluting biodegradable stent, a rotablator, a thrombus suction catheter, or a drug administration catheter. It is to be appreciated that the various embodiments and combinations of outer sheath, expandable outer sheath, occlusion device inner shaft, guide catheter and handle may be modified and adapted for use in combination with the variety of instruments 60 as well as those devices detailed in FIGS. 71 and 72.

FIG. 51B is a plan view showing the diagnostic instrument or a therapeutic instrument is inserted in the introducer sheath occlusion with perfusion device combination as in FIG. 51A. As indicated by the arrow, the slider 1556 of handle 1550 has been moved to withdraw the outer sheath 1580 from the occlusion with perfusion device 1500. As a result, the occlusion with perfusion device transitions into a deployed condition and is no longer against the outer wall of the guide catheter 5105. In this configuration, the occlusion with perfusion device would temporarily and reversibly occlude one or more branch vessels as further detailed with regard to FIGS. 44A, 44B, 44C, 57F, and 70. Still further, FIGS. 51A and 51B illustrate the use of a single access point for performing the therapy or intervention with instrument 60. Advantageously, only one vascular access point is used and the associated occlusion with perfusion device may be deployed as needed when contrast or other harmful agents are used during imaging is support of the intervention associated with instrument 5110.

FIG. 51C is a partial perspective view of an alternative introducer with occlusion device combination in a deployed condition as in FIG. 51B. The handle 1550 has a slider 1556 positioned to withdraw the outer sheath 1580 to place the occlusion with perfusion device into the deployed configuration as shown. The device catheter 5120, instrument 5110 and a guidewire 80 are visible at the distal end. The guide catheter 5105 is shown accessing the inner shaft lumen 1597 via the handle 1550 hemostasis valve 1599. The instrument 60 is coupled to the proximal portion of the device catheter 5120 (not shown). In this configuration, the occlusion with perfusion device 1500 when within the vasculature would temporarily and reversibly occlude one or more branch vessels as further detailed with regard to FIGS. 44A, 44B, 44C, 57F, and 70.

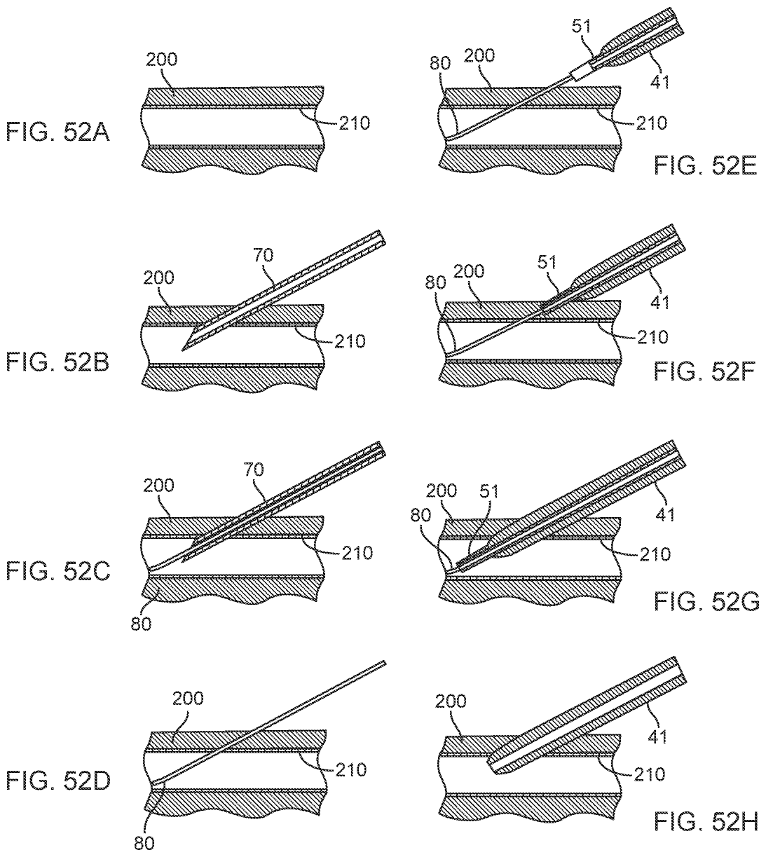

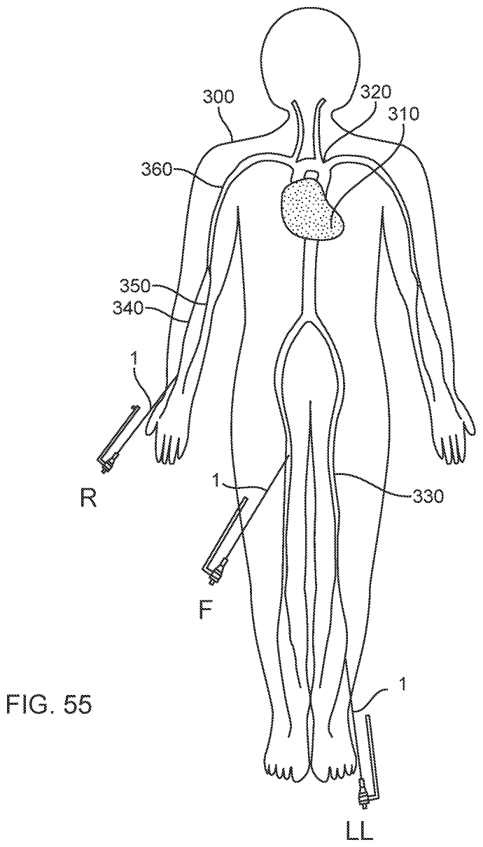

A procedure for percutaneously inserting the introducer sheath and occlusion with perfusion device combination 40 of this embodiment into a blood vessel will be specifically described below, referring to FIGS. 52A to 52H which those of ordinary skill will appreciate how they may be adapted for use of the combination embodiments for the various vascular access routes in FIG. 55.

FIGS. 52A to 52H are schematic views illustrating, in the order from 52A to 52H, the procedure of percutaneously inserting the introducer sheath 40 into a blood vessel.

The sheath tube 41 of the introducer sheath 40 is inserted through skin 200, shown in FIG. 52A, into a blood vessel 210 located beneath the skin 200. Specifically, first, as shown in FIG. 52B, a puncture needle 70 punctures the skin 200 toward the blood vessel 210. Next, as shown in FIG. 52C, a guide wire 80 is inserted through the lumen of the puncture needle 70 into the blood vessel 210. Subsequently, as shown in FIG. 52D, the puncture needle 70 is drawn out of (removed from) the blood vessel 210, with the guide wire 80 kept indwelling in the blood vessel 210. Next, as shown successively in FIG. 52E to FIG. 52G, the dilator tube 51 together with the sheath tube 41 (i.e., the dilator 51 positioned in the sheath tube 41) is inserted into the blood vessel 210 along the guide wire 80 and through the skin 200. Subsequently, as shown in FIG. 52H, the guide wire 80 and the dilator tube 51 are drawn out of the blood vessel 210, with the sheath tube 41 kept indwelling in the blood vessel 210. Thereafter, the diagnostic instrument or therapeutic instrument is inserted into the sheath tube 41.

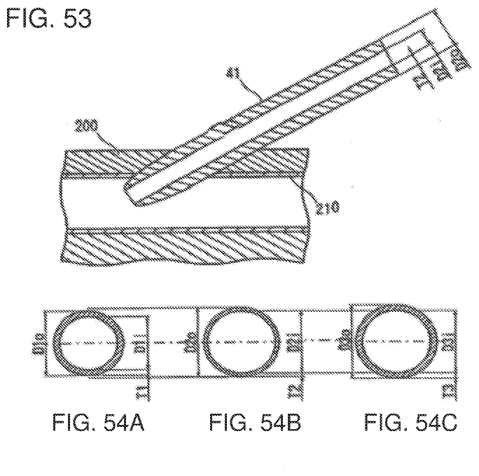

Various alternative introducer constructions are described with reference to FIGS. 53 and 54A to 54C.

FIG. 53 schematically illustrates a condition where the introducer sheath is indwelled in the blood vessel, while FIGS. 54A to 54C illustrate cross-sectional sizes of three exemplary kinds of introducer sheaths.

As shown in FIG. 53, the outer diameter D2o of the introducer sheath 40 is preferably set as small as possible, for helping to ensure relatively easy puncturing of the skin and a blood vessel, and for reducing invasiveness to vascular endothelium. In addition, the outer diameter D2o of the introducer sheath 40 is preferably set as small as possible, for accelerating the recovery of a punctured part after the treatment and for shortening the stanching time. On the other hand, the inner diameter D2i of the introducer sheath 40 is preferably set as large as possible, for permitting insertion of elongated bodies possessing large outer diameters. In some embodiments the introducer sheath of the combination introducer and occlusion device is 8 Fr, 7 Fr or 6 Fr.

FIG. 54B shows a cross-sectional shape/size of the introducer sheath according to this embodiment, and FIGS. 54A and 54C show cross-sectional shapes/sizes of introducer sheaths according to known constructions. Here, FIG. 54B shows the outer diameter D2o, the inner diameter D2i and the wall thickness T2 of the introducer sheath 40 according to this embodiment. FIG. 54A shows the outer diameter D1o, the inner diameter D1i and the wall thickness T1 of an introducer sheath according to a known construction. The known configuration of the introducer sheath shown in FIG. 54A is smaller in inner diameter than the introducer sheath 40 of this embodiment. However, both the outer diameter of the introducer sheath shown in FIG. 54A and the outer diameter of the introducer sheath 40 of this embodiment have almost the same size. FIG. 54C shows the outer diameter D3o, the inner diameter D3i and the wall thickness T3 of an introducer sheath according to another known construction. This known construction or configuration possesses a greater outer diameter than the introducer sheath 40 of this embodiment. And this known construction or configuration possesses an inner diameter substantially equal to the introducer sheath 40 of this embodiment.

The outer diameter D2o of the introducer sheath 40 shown in FIG. 54B has an outer diameter which is smaller than the outer diameter D3o and closer to the diameter D1o than D3o. In other words, the known introducer sheath shown in FIG. 54A corresponds to 5 Fr size. The phrase “an introducer sheath corresponding to 5 Fr size” means that the inner diameter of the introducer sheath can be inserted a device having an outer diameter of 5 Fr size. The outer diameter D2o of the introducer sheath 40 shown in FIG. 54B is equivalent to the outer diameter of 5 Fr size introducer sheath and other different sheath sizes as possible as described herein. In additional embodiments, any of the various embodiments of the outer shaft or sheath described herein may be modified to benefit from the variations described in FIGS. 53, 54A, 54B and 54C.

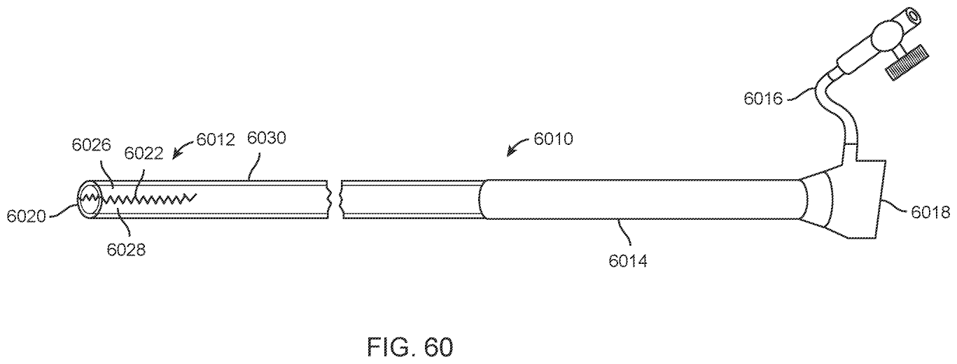

In still other aspects, those of ordinary skill will appreciate that “a device” accessing the vasculature using an embodiment of the combination introducer and occlusion device includes a diagnostic instrument or a therapeutic instrument. Moreover, these various principals of sheath design alone or in combination with those of FIGS. 60-68A may be applied to an introducer and occlusion with perfusion device embodiment for delivery of any device described above or in FIGS. 71 and 72. Additionally or optionally, the device as described herein may also be any or a part of a component, a device, a system or a procedure for use in transcatheter coronary repair or replacement such as, for example, transcatheter aortic valve repair or replacement (TAVR), transcatheter mitral valve replacement or repair (TMVR), and transcatheter tricuspid valve repair or replacement (TTVR).

A procedure for diagnosis or treatment of a coronary artery 320 by use of a diagnostic instrument or a therapeutic instrument through the introducer sheath and occlusion with perfusion device according to a suitable embodiment will now be described with reference to FIG. 6. In various alternative embodiments, one or more of these steps may be modified by one or more of the steps described in method 800 in FIG. 8.

FIG. 55 schematically illustrates a condition in which the introducer sheath and occlusion with perfusion device is inserted in a predetermined blood vessel of a patient 300.

In a case of vascular access point R, the diagnosis of the coronary artery 320 of the patient 300 is conducted by inserting a diagnostic instrument through a radial artery 340 or ulnar artery 350 into the coronary artery 320 of the patient 300. Still using access point R, treatment of the coronary artery 320 is conducted by inserting a therapeutic instrument through the radial artery 340 or ulnar artery 350 into the coronary artery 320, the procedure is carried out as follows.

First, an introducer having the dilator 50 inserted into and extending along the introducer sheath 40 is inserted into the radial artery 340, and then the dilator 50 is drawn out, with the introducer sheath 40 kept indwelling in the radial artery 340. It is also possible for the introducer to be inserted into the ulnar artery 350. Next, a diagnostic instrument having an outer diameter smaller than the maximum outer diameter permitted to be inserted into the introducer sheath 40 is inserted into the introducer sheath 40 and is inserted through the radial artery 340 into the coronary artery 320. A diagnosis is then made through the diagnostic instrument whether or not the coronary artery 320 is stenosed, and the diagnostic instrument is then drawn out. Further, when the coronary artery 320 is found stenosed, the introducer sheath 40 is kept indwelling in the radial artery 340, then, in this condition, a therapeutic instrument or a catheter permitting the therapeutic instrument to be inserted therein, which therapeutic instrument or catheter has the maximum outer diameter permitting insertion into the introducer sheath, is inserted into the introducer sheath 40, is inserted through the radial artery 340 into the coronary artery 320. When a catheter permitting insertion of the therapeutic instrument is inserted into the sheath, the therapeutic instrument is inserted into the catheter. Treatment is then performed. The diagnostic instrument having an outer diameter smaller than the maximum outer diameter permitting insertion into the introducer sheath 40 has an outer diameter smaller than the maximum outer diameter by, for example, 1 Fr size.

The above basic vascular access technique may be performed using an access procedure with the femoral artery (access route F) or using the vasculature of the lower limb such as the tibial artery or other suitable access route (access route LL). Alternative embodiments of the introducer with occlusion device may be sized appropriately according to the size of the access point vessels associated with access routes R, F and LL. The relative sizes of the outer sheath or introducer as well as the lumen of inner shaft of the occlusion with perfusion device are adjusted accordingly.

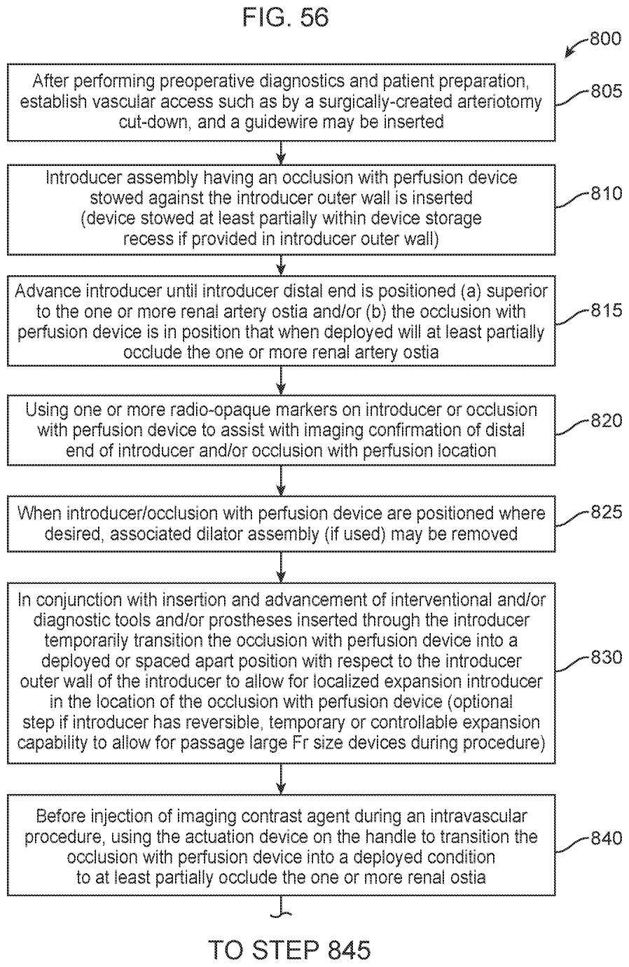

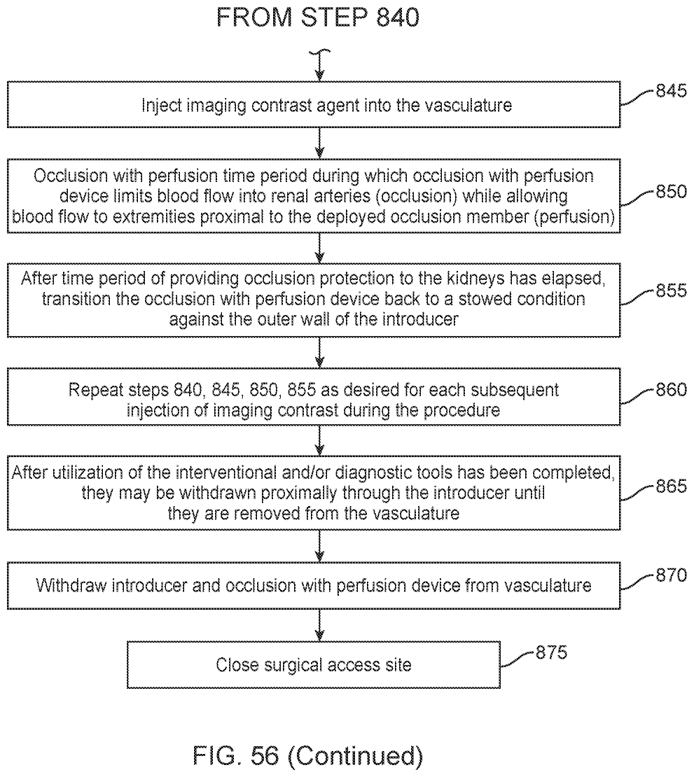

In various alternative embodiments, one or more of the vascular access steps may be modified by one or more of the steps described in method 800 in FIG. 56.

In the case where the introducer sheath is introduced through access point LL in a part near the back of the knee, the instep, or the heel to be set indwelling in a posterior tibial artery 390, a fibular artery 400, an anterior tibial artery 380, or a popliteal artery 370. Generally vascular access point LL in FIG. 55, a procedure the same as or similar to the above-mentioned procedure is used. Specifically, a diagnostic instrument is inserted through the posterior tibial artery 390, the fibular artery 400, the anterior tibial artery 380, or the popliteal artery 370 of the patient 300 into an artery that is the part to be treated, and the artery to be treated is diagnosed. Thereafter, a therapeutic instrument is inserted through the patient's posterior tibial artery 390, fibular artery 400, anterior tibial artery 380, or popliteal artery 370 into the artery to be treated. Then, the artery as the part to be treated can be treated. An occlusion with perfusion device may be deployed as needed during imaging as described herein.

This embodiment of the diagnostic/treatment method permits realization of a variety of effects.

In the case where diagnosis of a second artery of the patient 300 is conducted by inserting a diagnostic instrument through a first artery into the second artery and then treatment of the second artery of the patient 300 is conducted by inserting a therapeutic instrument through the first artery into the second artery, various effects are produced according to the size of the introducer sheath 40. For instance, in the case where the diagnosis of the coronary artery 320 of the patient 300 is conducted by inserting the diagnostic instrument through the patient's radial artery 340 or ulnar artery 350 (Access Route R) into the coronary artery 320 and subsequently the treatment of the coronary artery 320 is conducted by inserting the therapeutic instrument through the patient's radial artery 340 or ulnar artery 350 into the coronary artery 320, various effects are produced according to the size of the introducer sheath 40. In view of this, sizes of two kinds of introducer sheaths 40 will be specifically described.

In the case of an introducer sheath 40 having an inner diameter of 1.9 to 2.5 mm and a wall thickness of 0.05 to 0.19 mm, corresponding to the “6 in 5” mentioned above, the following effects are produced.

In a case where a stenosis is found upon diagnosis of the coronary artery 320 of a heart 310 of the patient 300 and treatment is conducted in succession to (following) the diagnosis, instead of conducting the treatment some other time, for example, the introducer sheath 40 already set indwelling in the radial artery 340 or ulnar artery 350 does not have to be replaced by another one with a larger inner diameter. These or other procedures may be modified for use in a femoral artery to access the aorta using an embodiment of the introducer with occlusion and perfusion device. (see generally Access Route F in FIG. 55).

In the case of conducting treatment in succession to diagnosis, in the previously used procedures, a sheath in which to insert and pass a device corresponding to an appropriate Fr size has had to be replaced by a sheath in which to insert and pass a therapeutic device corresponding to a larger Fr size. Such replacement of the sheath in the previously used procedures has produced various problems. The replacement of the sheath in the previously used procedures causes re-insertion of the sheath, leading to increased invasiveness to the patient 300 and a need for a sheath-replacing time. In addition, two sheaths are necessitated, which leads to increased cost.

In addition, the wall thickness, material composition and various other aspects of an introducer embodiment may be varied to accomplish the objective of delivery of large Fr size interventional devices in cooperation with modifications for low profile storage of an occlusion with perfusion device. It is to be appreciated that angiography catheters, intravascular ultrasound testing instruments and intravascular optical coherence tomography instruments can be applied or used as the intravascular instrument described herein. Still further, the introducer with occlusion device and method may be used to advantage with balloon catheters, drug-eluting balloon catheters, bare metal stents, drug-eluting stents, drug-eluting biodegradable stents, rotablators, thrombus suction catheters, or drug administration catheters or any other therapeutic intravascular instrument. Still further, guiding catheters and support catheters can be applied or used as the catheter. Thus, in using an embodiment of the introducer and occlusion with perfusion device, there are no specific restrictions as to the intravascular instrument or therapeutic instrument that may be utilized or deployed using the inventive introducer.

FIG. 56 sets forth an illustrative intravascular procedure 800 utilizing an embodiment of an introducer with an occlusion with perfusion device as described herein. Additional variation or different methods are possible and these steps may be modified depending upon a number of factors such as the procedure being performed, the organ or collateral structures being protected by the occlusion with perfusion device, the type of agent introduced and other factors related to advantageously employing the occlusion with perfusion device.

Referring to FIG. 56, after preoperative diagnostics and patient preparation (805), vascular access may be established, such as by a surgically-created arteriotomy cut-down, and a guidewire may be inserted, such as 0.035″ diameter guidewire. Optionally, in an embodiment using an 8 Fr sized outer sheath system a 0.018″ guidewire may be used. An introducer with an occlusion with perfusion device may be introduced (810). Next, advance introducer until introducer distal end is positioned (a) superior to the one or more renal artery ostia and/or (b) the occlusion with perfusion device is in position that when deployed will at least partially occlude the one or more renal artery ostia (815). One or more radio-opaque markers on the introducer or the occlusion device may be used to confirm position (820).

With the introducer assembly in place, the associated dilator assembly may be removed (825). Interventional and/or diagnostic tools and/or prostheses may be inserted through the introducer and occlusion device combination. In some configurations the introducer is capable of expansion wherein the expandable portion or expandable section 6875 is localized, such that after a relatively large device or implement is passed through and past a given portion of the introducer, that portion re-collapses, at least partially or completely. The occlusion with perfusion device may be spaced apart from the outer wall of the introducer or moved into a deployed configuration during insertion of intravascular devices so as to allow for localized expansion of an introducer, if so configured.

In conjunction with insertion and advancement of interventional and/or diagnostic tools and/or prostheses inserted through the introducer temporarily transition the occlusion with perfusion device into a deployed or spaced apart position with respect to the introducer outer wall of the introducer to allow for localized expansion introducer in the location of the occlusion with perfusion device (830).

Additionally or optionally, if introducer has reversible, temporary or controllable expansion capability to allow for passage large Fr size devices during procedure.

Before injection of imaging contrast agent during an intravascular procedure, using the actuation device on the handle to transition the occlusion with perfusion device into a deployed condition to at least partially occlude the one or more renal ostia (840).

Inject imaging contrast agent into the vasculature (845).

Occlusion with perfusion time period during which occlusion with perfusion device limits blood flow into renal arteries (occlusion) while allowing blood flow to extremities proximal to the deployed occlusion member (perfusion) (850).

After time period of providing occlusion protection to the kidneys has elapsed, transition the occlusion with perfusion device back to a stowed condition against the outer wall of the introducer (855).

Repeat steps 840, 845, 850, 855 as desired for each subsequent injection of imaging contrast during the procedure (860).

After utilization of the interventional and/or diagnostic tools has been completed, they may be withdrawn proximally (865). Thereafter, the introducer with occlusion with perfusion device is removed from the vasculature (870). Finally, the surgical access closed (875).

FIG. 57A is a cross section view of an embodiment of a combination access device having an occlusion with perfusion device 1500 in a stowed configuration between an inner wall of an outer sheath 1580 and a pocket 5745 of a modified dilator 5730. The device is shown within an aorta 5790 adjacent a pair of branch vessels 5792, 5794. Optionally, these vessels 5792, 5794 may be any of those detailed in FIG. 70. The views of FIGS. 57A and 57D provide a sense of scale for the change in dimensions between a stowed occlusion with perfusion device (FIG. 57A) and a deployed occlusion with perfusion device (FIG. 57D). The nominal diameter of the aorta is 20 mm. The diameter of the outer sheath containing the stowed occlusion with perfusion device in the FIG. 57A is 2.9 mm. Once the outer sheath is withdrawn and the perfusion device is deployed, the perfusion device spans the aorta as shown in FIG. 57D. In some embodiments, an occlusion with perfusion device will have a stowed condition within an outer sheath having a 2.9 mm with a deployed diameter of 20 mm. The diameter of the deployed occlusion device is 5, 6 or 7 times the diameter of the introducer.

The pocket 5745 may have a length corresponding to the length of an occlusion device from a position proximal to the coupling 1530 to the distal most portion of the occlusion device. In one embodiment, the length is 10 cm. The guide wire lumen 5732 of the dilator may be sized for a 0.018 inch guidewire. The lumen may have a diameter in the range from 0.035-0.0040 inches. In one embodiment, the recessed portion of the pocket sized to accommodate a 6 Fr guide catheter. The pocket dimensions may range from 0.095 to 0.1 inches.

FIG. 57B is a cross section view of FIG. 57A with arrows indicating that the outer sheath 1580 is being withdrawn proximally exposing the distal tip 5735 of the dilator 5730.

FIG. 57C is a cross section view of FIG. 57B with arrows indicating the continued proximal withdrawal of the outer sheath 1580. A distal portion of the occlusion with perfusion device is transitioned into a deployed configuration and is clear of the distal portion of the dilator pocket 5745.

FIG. 57D is a cross section view of FIG. 57C with arrows indicating the continued proximal withdrawal of the outer sheath 1580 to a final position proximal to the scaffold coupling 1530. The occlusion with perfusion device 1500 is transitioned into a deployed configuration and is clear of the dilator pocket 5745. An unattached portion of the scaffold covering 1685 is shown deflecting into an occluding the branch vessels 5792, 5794.

FIG. 57E is a cross section view of FIG. 57D with arrows indicating the proximal withdrawal of the dilator 5730 from the occlusion device 1500. The occlusion with perfusion device 1500, outer sheath 1580 and guide wire 80 remain in position within the aorta 5790 as before.

FIG. 57F is a cross section view of FIG. 57E with arrows indicating the distal advancement of a guide catheter 5105 along the guidewire 80 and within the inner shaft 1525 of the occlusion device 1500.

FIG. 57G is a cross section view of FIG. 57F with arrows indicating the distal advancement of the outer sheath 1580 along occlusion with perfusion device 1500. A proximal portion of the occlusion with perfusion device has transitioned into a stowed condition between an inner wall of the outer sheath 1580 and an outer wall of the guide catheter 5105.

FIG. 57H is a cross section view of FIG. 57G with arrows indicating the end of the distal advancement of the outer sheath 1580 along occlusion with perfusion device 1500. The occlusion with perfusion device 1500 is shown in a stowed condition between an inner wall of the outer sheath 1580 and an outer wall of the guide catheter 5105. In this configuration, blood flows along the aorta 5790 around the guide catheter 5105 and the outer sheath 1580. The outer sheath may also be configured as described herein having an expandable section in a distal end portion 6875 (See for example FIGS. 60-67E).

FIG. 58 is a cross section view of an alternative embodiment of a combination access device of FIG. 57A having an occlusion with perfusion device in a stowed configuration between an inner wall of an outer sheath 1580 and a pocket of a modified dilator 5730. The device is shown within an aorta 5790 adjacent a pair of branch vessels 5792, 5794. The dilator is modified to form a pocket 5745 by using a dilator shaft 5760 to couple the dilator tip 5735 to the dilator body 5740. The dilator shaft 5760 extends proximally into the outer sheath beyond the coupling of the occlusion device scaffold to the inner shaft. The length of the dilator shaft 5760 may be of a length sufficient for the therapeutic lengths 2 and 3 in FIG. 70 which will require longer covered scaffolds. The dilator shaft 5760 may provide column strength for such longer occlusion with perfusion devices.

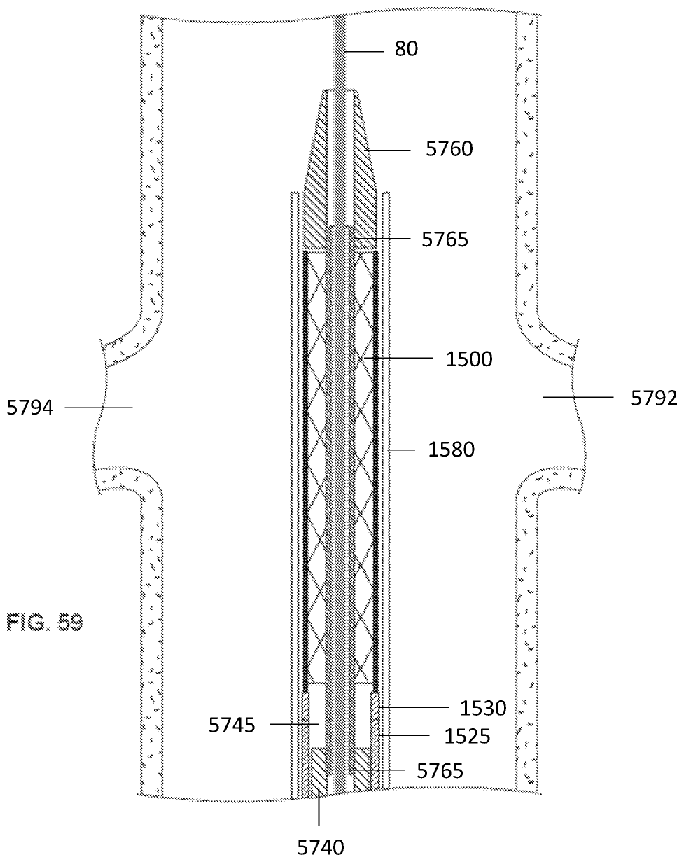

FIG. 59 is a cross section view of an alternative embodiment of a combination access device of FIG. 58. In this configuration, the dilator shaft 5765 used to couple the dilator tip 5735 to the body 5740 has a length sufficient for the length of the occlusion device to be stowed in the pocket with several millimeters of additional length. The additional length of the dilator shaft 5765 is used to extend the tube into the dilator lumen 5732 in the tip 5735 and into the body 5740. As before, in use, the occlusion with perfusion device in a stowed configuration between an inner wall of an outer sheath and a pocket of a modified dilator. The device is shown within an aorta 5790 adjacent a pair of branch vessels 5794, 5794. In this view the dilator is modified to form a pocket by using a dilator shaft 5765 to couple the dilator tip to the dilator body. The dilator shaft 5760, 5765 are secured within the dilator lumen 5732 using conventional means such as adhesive, heating, bonding, or other suitable technique.

In other alternative aspects of the inventive combination of an introducer and an occlusion device, there are provided a variety of alternative outer sheath or introducer configurations that will distend, expand or flex in order to translate an occlusion device into a stowed condition when using larger Fr sized guide catheters. Accordingly, when the guide catheter is present the flexible portion of the distal outer sheath may accommodate the occlusion device transition into a stowed configuration. A number of alternative configurations of expandable or distensible distal ends 6875 are described with regard to FIGS. 60-68C.

In order to further the combination aspect of embodiments of the invention, it is desirable to have an introducer sheath or outer shaft 1580 that is suitable for reconstraining (i) a deployed occlusion device, (ii) a large or awkwardly shaped surgical instrument and/or (iii) implantable devices after delivery such that they may be repositioned or removed from the body, including medical devices that are being removed from a body with a larger diameter than that of the introducer or outer sheath. Such additional capabilities advance the single vascular access point advantages of the combined occlusion with perfusion device with an introducer sheath or outer shaft having these additional capabilities. In an alternative aspect, the same introducer sheath or outer shaft may be used to reposition a device within the body to an alternative delivery site. An introducer sheath or outer shaft or sheath constructed according to this description may be used to recover a deployed occlusion device, a portion of a deployed occlusion device in the case of an occlusion device for multiple branch occlusion, deliver a medical device, surgical instrument, or biological sample. A modified outer sheath or introducer embodiment will have a reduced risk of splitting or tearing when a device is positioned within the introducer or outer sheath. As used here, the terms sheath, introducer sheath and outer shaft are used interchangeably within the context of use with an inner shaft with an occlusion device and a guide catheter or therapeutic catheter accessing the vasculature via the single access point via the inner shaft and extending through the occlusion device.

According to one embodiment, a distal tip of an introducer sheath or outer shaft is constructed to expand radially and thus facilitate the retrieval and repositioning of surgical tools, implantable devices, or biological matter that have a larger diameter than the unexpanded diameter of the introducer sheath or outer shaft. The distal end of the introducer sheath or outer shaft may be formed with either a single layer or multiple layers of material which may be the same or different from the materials comprising the rest of the introducer sheath or outer shaft. In one embodiment, the distal end of the introducer sheath or outer shaft may have one or more straight or curved generally longitudinally-oriented slits. The slits extend through the thickness of one or more layers of the introducer sheath or outer shaft. During delivery of a device, the slits may be closed or open depending on desired delivery characteristics. If the device requires removal or repositioning, the slits in the introducer sheath or outer shaft separate and the introducer sheath or outer shaft diameter expands if necessary as the device is retrieved into the introducer sheath or outer shaft. An elastomeric layer holds the sliced portions of the introducer sheath or outer shaft together and provides an expandable layer so that the introducer sheath or outer shaft remains a single piece. The slits may extend longitudinally from the distal end to a location up to 15 cm along the length of the introducer sheath or outer shaft or more. Alternatively, the slits may begin at a location slightly away from the distal end and continue longitudinally for up to 15 cm along the introducer sheath or outer shaft or more.

In another embodiment, one or more zig-zag slits may be provided longitudinally along a length of the distal end of the introducer sheath or outer shaft and in a direction perpendicular to the radial axis of the introducer sheath or outer shaft, or it can have some angle relative to a perpendicular orientation, or they can have an overall curved shape. The zig-zag configuration of the slits may include straight cuts or separations in the introducer sheath or outer shaft. The zig-zag cuts also may be rounded at the peak and/or the valley of the cut, and/or along the length of the cut. In a preferred form, the size of the zig-zag slits are constructed so that in an expanded configuration (e.g., when a device has been retrieved) the teeth of opposing sides of the zig-zag do not completely separate. Thus, the introducer sheath or outer shaft minimizes the likelihood of a longitudinal tear of the elastomeric material, if present. It is desirable that the entire device that has been inserted into the introducer sheath or outer shaft remain in the introducer sheath or outer shaft and not extend through any perforations or tears in the introducer sheath or outer shaft.

The formations described above may be used together and other formations may be used to allow for radial expansion of the introducer sheath or outer shaft as the device is being positioned within the introducer sheath or outer shaft. These formations may or may not require longitudinal contraction. These formations can be present along a portion or the entire length of the sheath tip. Other materials can be added to the sheath tip, such as wires for strength, coatings to change friction characteristics, and coatings of a different durometer, or, the device can be made to have a minimal number of parts and portions.

The introducer sheath or outer shaft can be an introducer through which surgical instruments and implantable devices such as stents, filters, occluders, valves, or other devices are inserted into a living body. The introducer sheath or outer shaft can also be a retriever through which tissue or other biological matter, surgical instruments, and implantable devices are withdrawn from a living body. The cut of the introducer sheath or outer shaft material that forms the slits may be aligned with the radial axis or may be slanted or curved. The cut may be formed from a sharp object, such as a knife, or alternative methods may be used to form the slits.

In another embodiment, the introducer sheath or outer shaft or sheath may have a distal end that is partially or wholly comprised of braided material. In such a device that uses a braided configuration, the longitudinal length shortens as the radius expands. This embodiment has the advantage that individual segments of the introducer or outer sheath are not separated as the introducer or outer sheath expands radially.

A radially expandable distal end 6875 of an introducer sheath or outer shaft allows surgical instruments, biological matter, and implantable devices, including such devices as may be folded, compressed, or loaded in the sheath in a specialized manner such that the device can be introduced through a smaller diameter delivery sheath than otherwise possible, to be more easily deployed upon delivery to the desired site within the body. In a specific implementation, such a modified outer sheath may be advantageously employed to recover, collapse an occlusion with perfusion device onto an outer wall of a guide introducer or outer sheath within the occlusion with perfusion device. As a result, a radially expandable distal end 6875 of an introducer sheath or outer shaft 1580 may also allow and/or facilitate retrieval of surgical instruments and implantable devices, including devices that unfold or expand or otherwise deploy in some way after delivery within the body through a guide catheter, and within the introducer sheath or outer shaft, and occlusion with perfusion device is withdrawn. The expandable distal end 6875 can accommodate more easily the volume of a partially or wholly deployed device, and can overcome snags resulting from the geometry of a partially or wholly deployed device, reducing trauma to the vessel through which such instruments or implantable devices must be withdrawn. Once a device, such as a deployed occlusion with perfusion device is retrieved into the sheath or outer shaft, the sheath tip can further aid in the complete recovery of a device by acting to compress the device. It is desirable that an expandable distal end 6875 of an introducer sheath or outer shaft 1580 accommodate an article with a larger dimension than that of the outer shaft/outer sheath.

An outer sheath 1580 can expand radially at its distal end to accommodate an element (e.g., medical device) that is larger than the diameter of the outer sheath. At times it is desirable, sometimes necessary, to remove or reposition a medical device that has been previously deployed. An introducer shaft or outer shaft as described here allows a device to be removed or repositioned by expanding to accommodate the device as the device is brought within the introducer sheath or outer shaft. According to some embodiments, the introducer sheath or outer shaft is configured to reduce the possibility of tearing the elastomeric layer longitudinally along the introducer or outer sheath by the edges of a surgical instrument or implantable device being removed or repositioned.

Referring to the drawings, wherein like reference numerals designate identical or corresponding parts throughout the several views, and more particularly to FIG. 60 thereof, an introducer sheath or outer shaft 6010 is illustrated with a distal end portion 6012. The introducer sheath or outer shaft according to this embodiment is adapted to be introduced into the vasculature in a normal procedure as known to those skilled in the art. The expandable distal end portion 6012 can expand radially when something having a larger diameter than its normal diameter is introduced into the distal end. The introducer sheath or outer shaft 6010 includes a hub portion 6014 and side tube 6016 which leads into the hub portion 6014. A medical instrument or implantable device to be inserted into a patient is placed through a proximal end 6018 and is intended to exit the introducer sheath or outer shaft 6010 at a distal end 6020. When the introducer sheath or outer shaft 6010 is used to remove or reposition an implantable device the device enters the introducer or outer sheath at the distal end 6020. The implantable device placed, removed or repositioned through the introducer sheath or outer shaft 6010 may be a medical device, including, e.g., stents, filters, occluders, valves or other devices, or a delivery element to deliver a medical device, including stents, filters, occluders, valves, or other devices, into a patient's body.

The introducer sheath or outer shaft 6010 can be various lengths, such as between 10 cm and 100 cm. The introducer or outer sheath can be longer or shorter as necessary for a particular application. The diameter of the introducer or outer sheath is typically between 5 and 20 French. Additionally, some devices are accessed using 24 Fr. Improvements continue and the sizes of devices that will access the vasculature using the inventive introduced and occlusion device combination will be expanding. Of course, the introducer sheath or outer shaft could have a larger or smaller diameter as a particular application warranted. Typical wall thickness of the introducer or outer sheath 6010 can vary greatly depending on the material selected and the length of the introducer sheath or outer shaft.

As illustrated in FIG. 60, the distal end 20 of the introducer sheath or outer shaft 6010 is expandable because of zig-zag shaped slit 6022 disposed on the distal end of the introducer sheath or outer shaft. A second zig-zag slit (not viewable) is disposed on the other side of the circumference of the introducer sheath or outer shaft. The zig-zag slits create two introducer sheath or outer shaft portions 6026 and 6028 with a generally semi-circular cross-section along the length of the zig-zag slits. A third zig-zag slit can also be provided to divide the circumference into three sections, and further slits could be provided. In each case, the slits can be centered equally spaced around the circumference, e.g., every 120 degrees for three slits, or they can be spaced at unequal intervals, e.g., at 90 to 180 degrees for three slits. As described in more detail below, when a device is introduced into the distal end of the introducer sheath or outer shaft to be removed or repositioned, the slits allow the introducer sheath or outer shaft portions 6026 and 6028 to separate to accommodate the device. A clear (as illustrated) elastomeric layer 6030 is on the outside of the introducer sheath or outer shaft and enables the introducer sheath or outer shaft to have the required structural integrity.

The elastomeric layer may be disposed on the inside surface of the introducer sheath or outer shaft or on the outside surface of the introducer sheath or outer shaft or both. The layers of the introducer or outer sheath are bonded together, such as through heat bonding, adhesives, or other suitable methods to join the two or more layers. If the elastomeric layer is disposed on the outer surface of the introducer or outer sheath a heat shrink tube may be used. Although the thickness of the layer may vary depending on the needs of a particular application and the material selected, the thickness may be between about 0.001 and 0.025 inches (25 to 625 microns), preferably between about 0.006 and 0.008 inches (150 to 200 microns). Materials for the elastomeric outer cover may include silicone, polyurethane, or polyether-amide block copolymer, such as a material known as Pebax. The elastomeric layer(s) allows the introducer or outer sheath portions 6026 and 6028 to expand as much as needed to recapture or reposition the device. The elastomeric outer cover can be flush with an inner wall at the distal end of the introducer sheath or outer shaft, or the outer cover can extend beyond the inner wall a short distance to create an overhang that provides a less stiff and “softer” end. This softer tip can help to guide a divide that may have coils or other structures that could get caught if brought back into contact with a stiffer conduit. This overhang would typically have a length of about 0.005 to 0.5 inches (0.125 to 12.5 mm) and preferably about 0.1 inches (2.5 mm), and a thickness of about 0.005 to 0.1 inches (0.125 to 2.5 mm), and preferably about 0.02 to 0.04 inches (0.5 to 1.0 mm). In addition to the end portion, other sections of the introducer or outer sheath can include multiple layers.

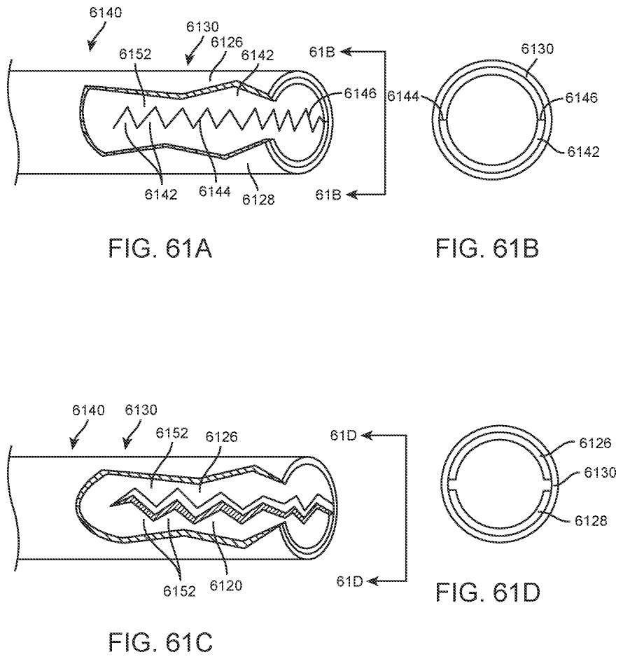

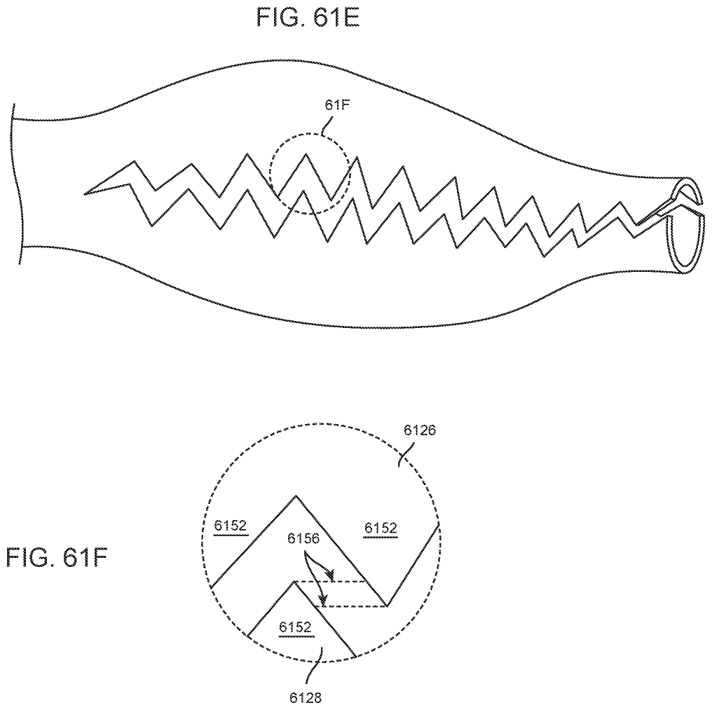

FIGS. 61(a) and 61(b) illustrate a distal end portion 6140 of an introducer sheath or outer shaft. The illustrated embodiment includes a two-wall structure comprised of an elastomeric cover 6130 surrounding a relatively high stiffness inner wall 6142 (compared to the stiffness of the outer wall). The inner wall has two slits 6144, 6146 extending in a zig-zag pattern along a longitudinal direction at the distal end of the introducer sheath or outer shaft. The material for the inner wall may include high density polyethylene (HDPE), high-stiffness polyether-amide block copolymer or, high stiffness polyurethane. The zig-zag pattern may extend longitudinally up to 15 cm or more along the length of the distal end portion 6140 of the introducer sheath or outer shaft.

The zig-zag pattern forms tooth shapes 6152 along the length of the zig-zag pattern. The shapes may be triangular as shown or, alternatively, rectangular, semi-circular or irregular. As depicted in FIG. 61(a), zig-zag slits of the inner wall preferably result in teeth with acute angles and teeth of height equal to one-quarter of the circumference, although the height could vary. Tooth geometry may be variable along the length of the distal end portion 6140 of the introducer sheath or outer shaft. For example, larger teeth may be provided at the distal end of the introducer sheath or outer shaft and smaller teeth may be provided towards the proximal end. The geometry of the teeth may change along the length of the slit such that the leading edge of the tooth has an angle to provide a more longitudinal profile. Thus, teeth sizes, widths or shape may change along the length of the tube tip or may change into one of the various slits types discussed below. Of course, more than two longitudinally extending zig-zag slits may be formed at the distal end portion 6140 of the introducer or outer sheath. If more than two slits are created, the spacing may be equal along the circumference of a cross-section or, alternatively, the spacing can vary. Varied spacing of the slits may be helpful if a device has an irregular geometry.

FIGS. 61(c) and 61(d) illustrate the distal end portion 6140 of the introducer sheath or outer shaft in a slightly expanded configuration. The introducer or outer sheath 6126, 6128 portions with semicircular cross-sections are slightly spread apart and allow for a device with a larger diameter to be inserted into the introducer sheath or outer shaft than would be able to absent the longitudinal slits. The elastomeric layer 6130 shown partially removed in FIG. 61(c). FIG. 61(d) illustrates the stretching of the elastomeric layer when the introducer or outer sheath portions 6126 and 6128 are separated. The slits provide additional flexibility of the inner wall to facilitate expansion, while maintaining longitudinal or column stiffness to inhibit buckling. In the preferred embodiment, the inner and outer layers are bonded in a manner that allows slippage at the teeth edge of the inner layer so that the stress of expansion is distributed to a larger portion of the elastomeric cover.

Referring to FIGS. 61(e) and 61(f), when introducing a device into the introducer sheath or outer shaft after it has been deployed there is a possibility that a portion of the device may have an edge that is sharp enough to tear the elastomeric material as the device is brought into the introducer or outer sheath. The configuration of the teeth that extend in a zig-zag pattern is designed to prevent puncture or tearing of the elastomeric cover. That is, the teeth are designed to be long enough to overlap as much as possible during the introduction of the device. As illustrated in FIG. 61(f), it may be advantageous to extend the elastomeric material beyond the distal end of the stiffer layer. This extension assists in the retrieval of the device by guiding or “funneling” the device into the introducer or outer sheath. The extension may be approximately 0.10 in (0.25 cm). Of course, shorter or longer extensions may be used depending on specific situations. As illustrated in FIG. 61(f), the overlap of the teeth 6152 by the distance designated by reference numeral 6156 minimizes the possibility that a sharp edge of a device will tear the elastomeric layer as it is drawn into the introducer sheath or outer shaft. Of course, the teeth may be constructed so that they separate sufficiently when a device is introduced into the introducer sheath or outer shaft so that the distance 6156 may be reduced to zero. It is also contemplated that the teeth may be designed not to overlap when an object with a much larger diameter is introduced into the introducer sheath or outer shaft. The overlapping ends of the teeth are helpful to make sure that the elastomeric layer is not torn by any sharp edge.

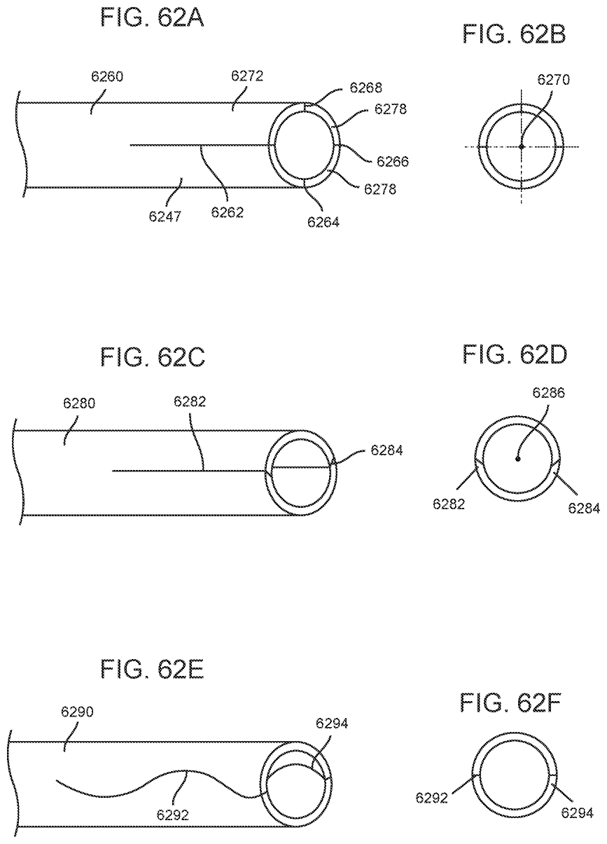

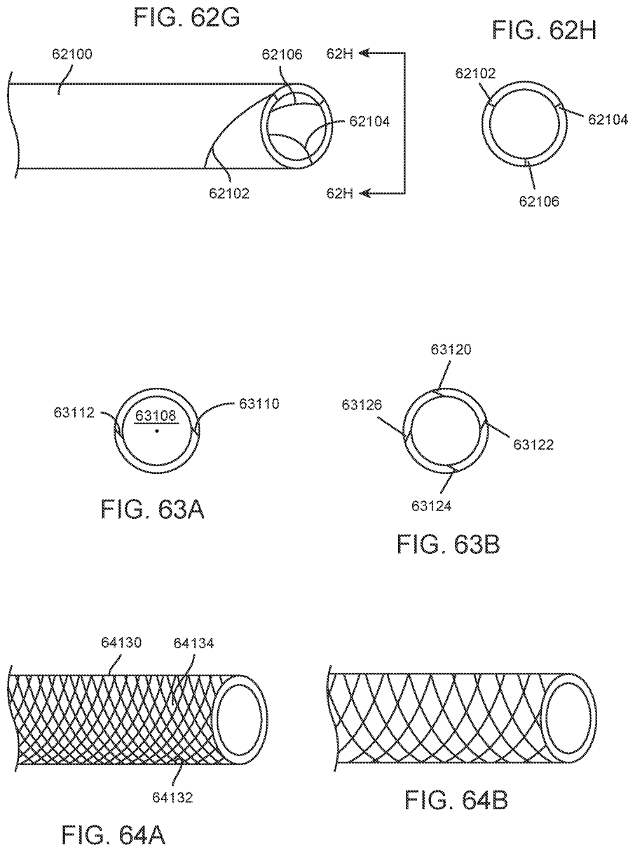

FIGS. 62(a) through 62(h) illustrate other aspects that may be incorporated into introducers or outer sheaths described herein. For clarity of illustration, the elastomeric layer has not been illustrated, but may or may not be present. Specifically, FIGS. 62(a) and 3(b) show the distal end portion 6260 with four slits 6262, 6264, 6266, and 6268 disposed longitudinally along a length of the distal end. The length of the slits may be up to 15 cm or more. The slits create introducer or outer sheath quarter sections 6272, 6274, 6276 and 6278 which separate and contain a device within the distal end. As illustrated in FIG. 62(b), the slits may extend in a direction radial to the center 6270 of the cross-section of the tube. This is a simple, easy to create geometry. FIGS. 62(c) and 62(d) illustrate an alternative geometry for the slit. Specifically, a distal end portion 6280 may be provided with two slits 6282 and 6284 that are oriented at an angle such that they do not intersect the center 6286 of a cross section of the introducer or outer sheath end portion 6280. Slots of this configuration may assist in keeping the elastomeric layer bonded to the high durometer (inner) layer of the introducer sheath or outer shaft, or, when still overlapped, minimize tearing of the elastomeric layer, if present. FIGS. 62(e) and 62(f) are still other alternative embodiments. As illustrated a distal end portion 6290 has two slits 6292 and 6294 that extend from the distal end of the introducer sheath or outer shaft. The slits 6292 and 6294 are curved or wavy along the length. The curved slits are relatively easy to construct and may provide advantages over the straight slits by reducing the possibility that sharp edges of a device would tear the elastomeric layer and otherwise facilitating delivery or recovery of an instrument or device. FIGS. 62(g) and 62(h) illustrate still further another embodiment. Here, a distal end portion 62100 includes helical slits 62102, 62104 and 62106.

FIGS. 63(a) and 63(b) illustrate the end view of an introducer sheath or outer shaft having alternative configurations for the orientation of the slits that may be used to create any of the slits previously mentioned. FIG. 63(a) has two slits 63110 and 63112 that are oriented in a manner shown. Similarly, FIG. 63(b) illustrates four slits 63120, 63122, 63124, and 63126 that are cut into the introducer sheath or outer shaft in the manner illustrated. Each of these slit configurations can be varied by the number of slits in the introducer sheath or outer shaft and the orientation of the slit. The slit configurations can be applied to each of the embodiments described elsewhere herein.

In another embodiment, the expandable introducer sheath or outer shaft end portion 64130 includes a wall 64132 formed by braided material 64134 as illustrated in FIGS. 64(a) and 64(b). The braid 64134 has one or more threads of high-stiffness material knitted or woven together as described above in the various embodiments in FIGS. 14A-15D. In one specific embodiment, the braided distal end may be approximately the same size as or smaller than the rest of the sheath tube. Braided material has the advantage of readily expanding in the radial direction. This advantage is used to accommodate the introduction of a device into the distal end of the introducer sheath or outer shaft. As the introducer sheath or outer shaft radially expands to accommodate a device, the braided material contracts longitudinally, i.e. axially, as depicted in FIG. 64(b). Longitudinal compression of the distal end of the introducer sheath or outer shaft may be achieved by the positive force of the occlusion device, tissue sample, surgical instrument, or implant device being withdrawn into the sheath tip. Alternatively, the longitudinal contraction of the distal end of the introducer or outer sheath may be produced by the positive action of a control rod or contraction cable. The braided expandable distal end of the introducer sheath or outer shaft illustrated in FIGS. 64(a) and 64(b) may or may not include an elastomeric outer cover.

Features of the embodiments described here include the following: (a) an outer sheath expansion zone or an expandable sheath tip facilitates the deployment and retrieval of the various embodiments of occlusion with perfusion devices described herein, surgical instruments, implantable devices, and biological matter; alone or in combination with (b) use of the expandable sheath tip to partially deploy, expand or inflate an implantable device or surgical instrument before delivery of such implantable device or surgical instrument is specifically envisioned. The sheath tip radially expands to more easily accommodate implantable device or surgical instrument volumes and overcome any device or instrument geometry that may tear an elastomeric sleeve. The sheath tip may or may not be accompanied or enhanced by the addition of other materials such as braids, different tubing, or coatings. The elastomeric material, when present, expands such that the implant will be fully or partially encapsulated within the tip. The elastomeric material, when present, also serves to ensure a controlled and consistent expansion of the tip geometry. In addition to the containment of the retrieved device and protection against cut sheath tip areas, the elastomeric material, when present, may extend past the tip of the sheath to form a highly flexible ring that corrects snags, ensuring the successful entry of the device into the sheath tip.

Once the device is retrieved, the material continues to aid in the complete recovery by compressing the implant to facilitate any remaining size discrepancy between the retrieved device and the dimensions of the full length of the sheath. The expandable sheath tip preserves rigidity, column strength, and stiffness where necessary.

In other configurations of introducers or outer sheaths, combinations of the above embodiments are possible. For example, one embodiment includes a high-durometer inner wall with a longitudinally-oriented zig-zag slit, having a cover comprised of a low-durometer braided material. Additionally, the slits may extend the entire length of the introducer sheath or outer shaft so that a device may be pulled through the length of the introducer sheath or outer shaft. Numerous modifications and variations of the present inventions are possible in light of the above teachings. Although the embodiments have been described in detail for the purpose of illustration, it is understood that such detail is solely for that purpose, and variations can be made by those skilled in the art without departing from the spirit and scope of the inventions.

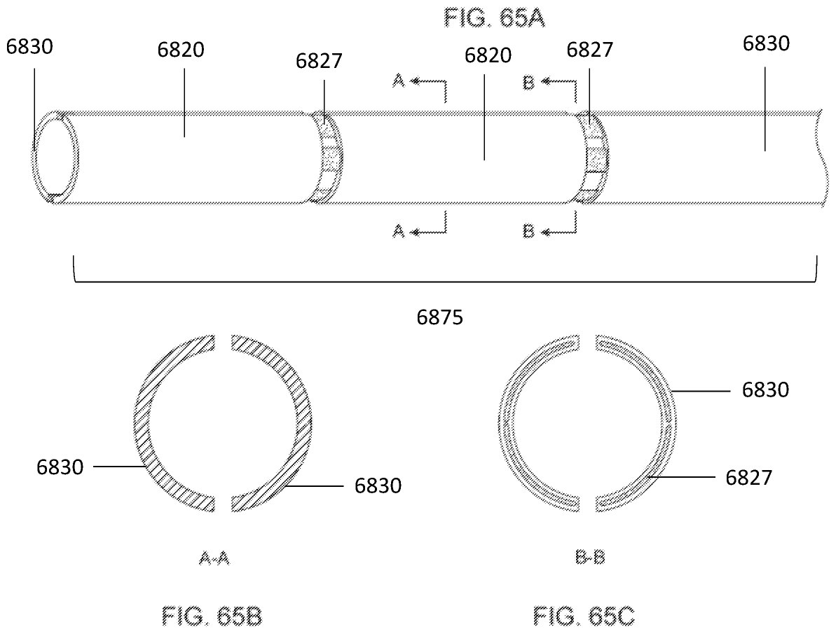

FIGS. 65A-68C provide additional variations to an outer sheath having an expandable distal end or a distal end expansion zones such as those illustrated and described above with regard to FIGS. 60-64B. Similar uses, design principals and applications of the above embodiments of an introducer sheath or outer shaft 6010 is illustrated with a distal end portion 6012 are applicable to the distal end expandable zone 6875 in these various configurations. As detailed before, the introducer sheath or outer shaft according to this embodiment is adapted to be introduced into the vasculature in a normal procedure as known to those skilled in the art. The outer sheath expandable distal section 6875 is similar in characteristic to those of the embodiments of section expandable distal end portion 6012 in that common capability to expand radially when something having a larger diameter than its normal diameter is introduced into the distal end.

FIG. 65A is a perspective view of three sections of an embodiment of an outer sheath expansion zone 6875. The distal end expandable section 6875 includes three sections 6820. Each section 6820 includes two segments 6830. Flexible joints or couplings 6827 are visible between segments 6830 of adjacent sections 6820.

FIG. 65B is an end view of the sheath expansion zone 6875 of FIG. 65A taken along section A-A. This view shows a cross section of two segments 6830 within a section of the outer sheath expansion zone 6875. In this embodiment, the circumference of section 6820 is formed by two semi-circular segments 6830.

FIG. 65C is an end view of the sheath expansion zone 6875 of FIG. 65A taken along section B-B. This view shows a cross section of two segments 6830 within a section 6820 of the outer sheath expansion zone 6875 where a flexible joint 6827 attaches within, on or within a portion of a segment 6830.

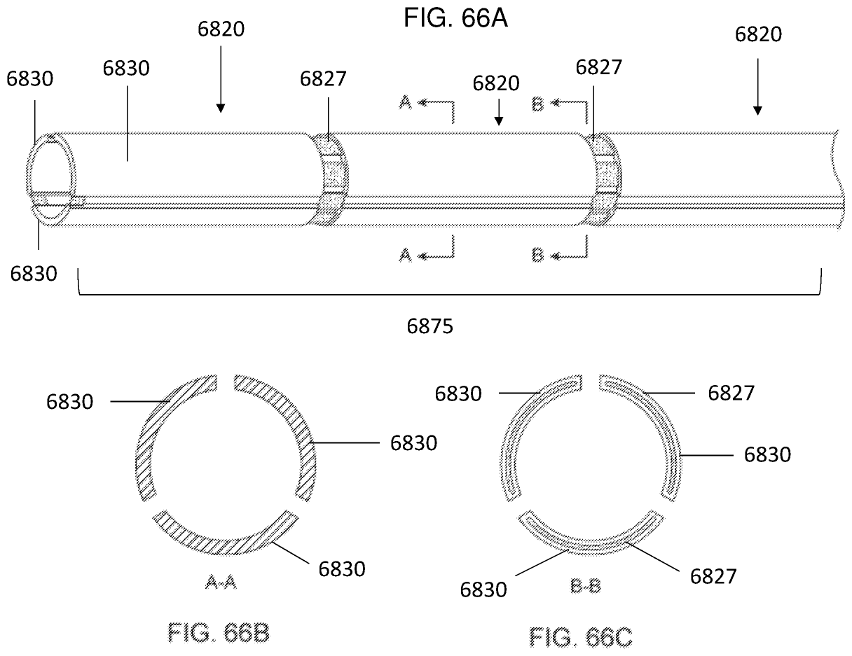

FIG. 66A is a perspective view of three sections 6820 of an embodiment of an outer sheath expansion zone 6875. In this embodiment, each section 6820 includes three segments 6830. Flexible joints or couplings 6827 are visible between segments 6830 of adjacent sections 6820.

FIG. 66B is an end view of the sheath expansion zone 6875 of FIG. 66A taken along section A-A. This view shows a cross section of three segments 6830 within a section 6820 of the outer sheath expansion zone 6875. In this embodiment, the circumference of section 6820 is formed by three segments 6830 have an arcuate shape of about 120 degrees.

FIG. 66C is an end view of the sheath expansion zone 6875 of FIG. 66A taken along section B-B. This view shows a cross section of three segments 6830 within a section 6820 of the outer sheath expansion zone 6875 where a flexible joint 6827 attaches within, on or within a portion of a segment.

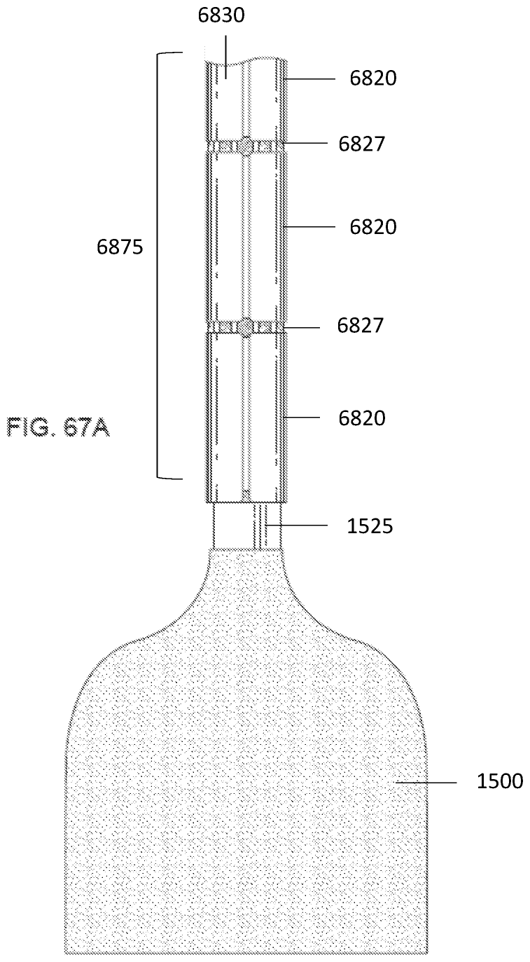

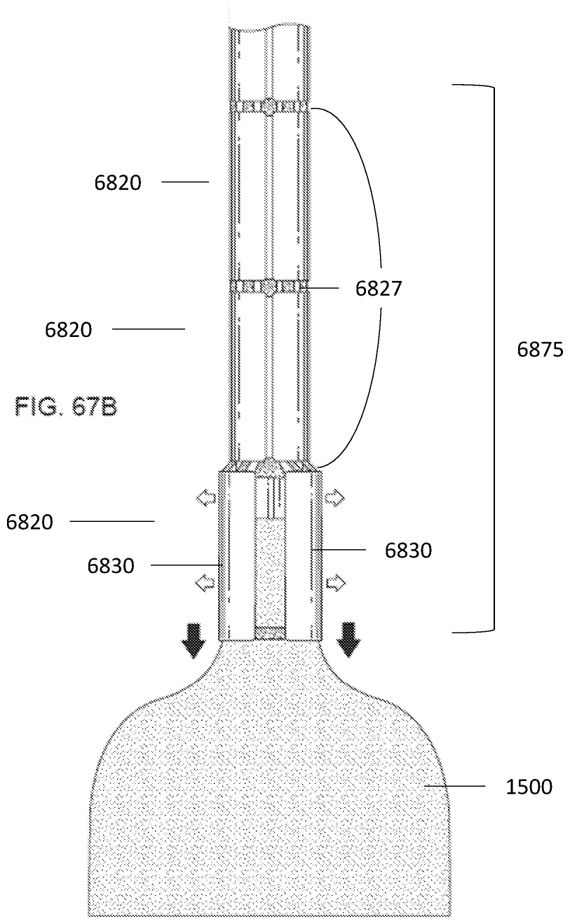

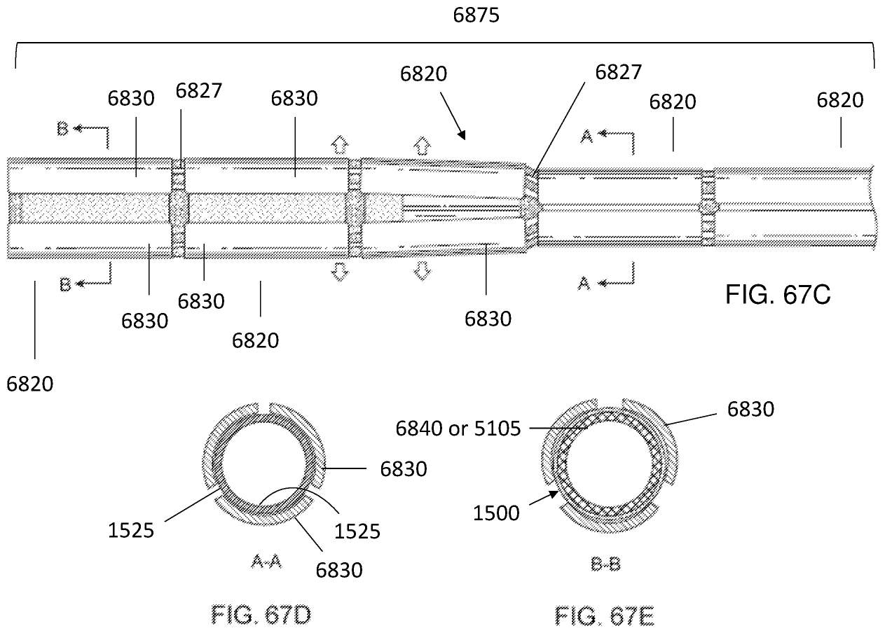

FIG. 67A is a side view of a sheath expansion zone 6875 in a non-extended configuration against the outer wall of the inner shaft 1525. The distal end of the distal most section 6820 of the outer shaft expansion zone 6875 is proximal to an occlusion with perfusion device 1500. The occlusion with perfusion device 1500 is shown in a deployed configuration.

FIG. 67B is a side view of the sheath expansion zone 6875 of FIG. 67A with arrows indicating the distal advancement of the outer sheath 1580. Also shown in this view are arrows indicating the relative displacement of segments 6830 and flexible joints 6827 in a section 6820 that has captured a proximal portion of the deployed occlusion with perfusion device 1500.

FIG. 67C is a side view of the sheath expansion zone 6875 of FIG. 67B after the distal advancement of the outer sheath 1580 to capture the occlusion with perfusion device 1500. Arrows indicate the relative displacement of segments 6830 and flexible joints 6827 in the sections of the expansion zone 6875 that have captured the occlusion with perfusion device 1500 of FIG. 65A. When captured in this stowed condition, the occlusion with perfusion device 1500 is between an outer wall of the guide catheter 5105 and an inner wall of the outer sheath expansion zone 6875.

FIG. 67D is an end view of the sheath expansion zone 6875 of FIG. 67C taken along section A-A where the expansion zone 6875 remains against the outer wall of the inner shaft 1525 (i.e., the expansion zone 6875 remains in an unexpanded condition). This view shows a cross section of three segments 6830 within a section 6820 of the outer sheath expansion zone 6875 against the outer wall of the inner shaft 1525.

FIG. 67E is an end view of the sheath expansion zone 6875 of FIG. 67C taken along section B-B. This view shows a cross section of three segments 6830 within a section 6820 of the outer sheath expansion zone 6875 that has captured the occlusion with perfusion device 1500. In this configuration the occlusion with perfusion device 1500 is in a stowed configuration between the outer wall of the guide catheter 6840 or 5105 and the inner wall of the outer sheath expansion zone 6875. In this configuration, the inner wall of the outer sheath 1580 is provided by the inner wall of each of the three segments 6830. The inner wall of an embodiment of the expandable distal end portion 6875 may vary depending on the specific construction of the particular expandable section being implemented.

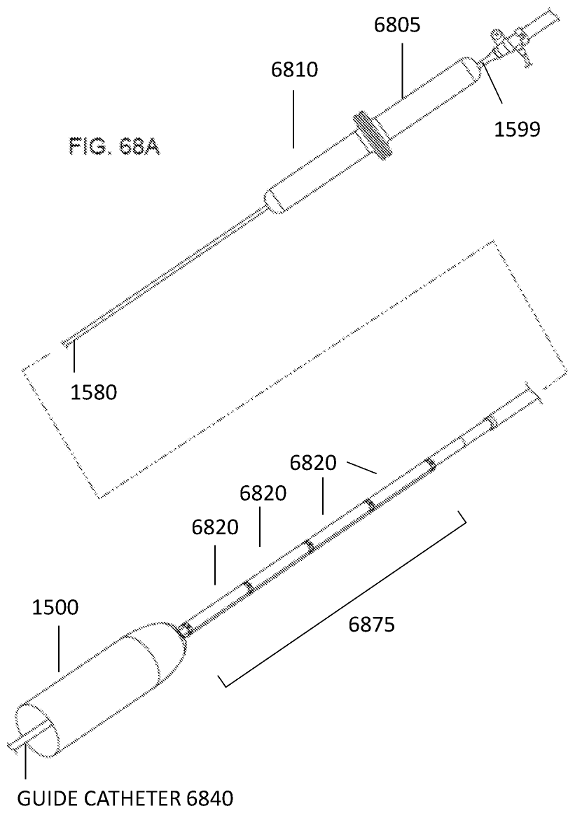

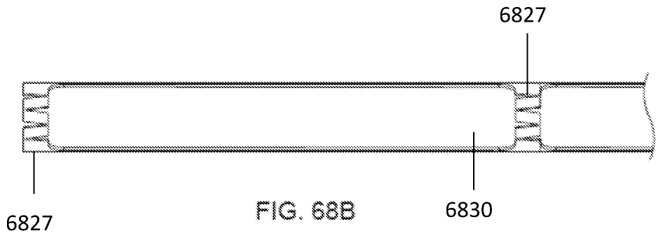

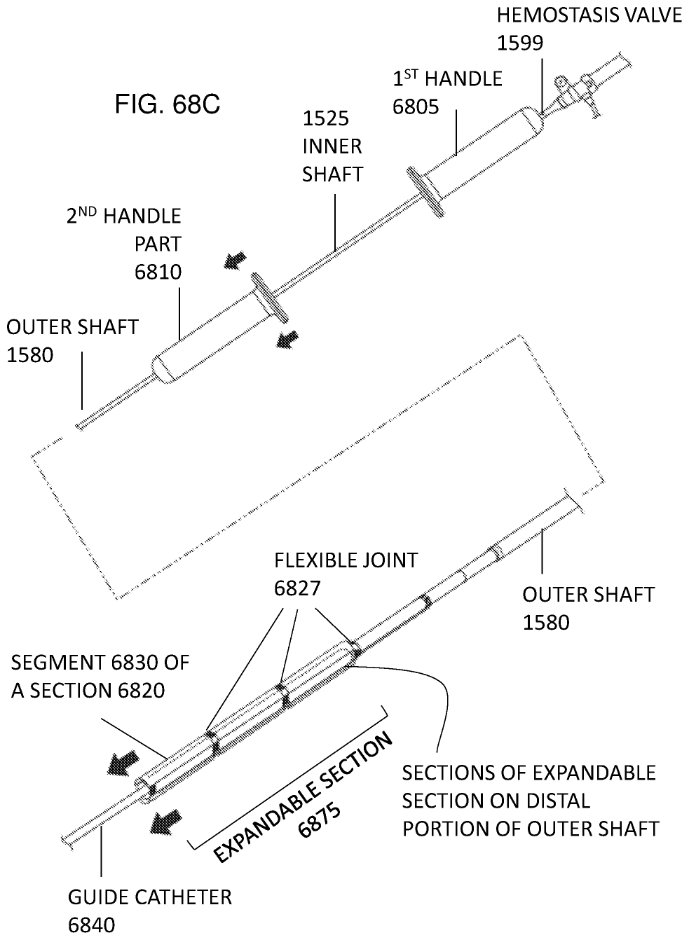

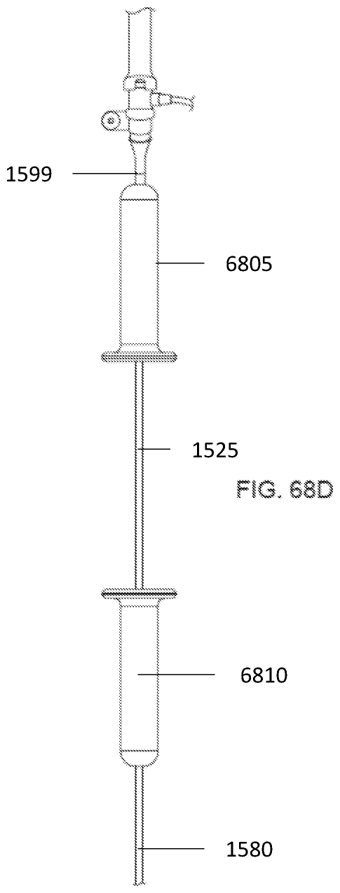

FIG. 68A is a perspective view of a combination occlusion with perfusion device 1500 having an outer sheath 1580 with an expansion zone 6875. The outer sheath expansion zone includes four sections 6820. There is an outer sheath handle 6810 and inner sheath handle 6805 at the proximal end. An occlusion with perfusion device 1500 is shown beyond the distal end of the outer sheath 1580 in a deployed configuration. A guide catheter 6840 is shown within the interior of the deployed occlusion with perfusion device 1500. FIG. 57F illustrates a cross section view of a similar arrangement of a guide catheter within a deployed occlusion with perfusion device. When the handles 6810, 6805 are together, the occlusion with preclusion device 1500 is deployed as shown.

FIG. 68B is a side view of one complete section 6820 and a partion section 6820 of the expansion zone 6875 of FIG. 68A. A segment 6830 has a flexible coupling 6827 on each end. The flexible coupling 6827 on the proximal end is shown joined to an adjacent segment 6830.

FIG. 68C is a perspective view of the combination occlusion with perfusion device having an outer sheath 1580 with an expansion zone 6875 of FIG. 68A. Arrows indicate the movement of the second handle part or outer sheath handle 6810 relative to the first handle part or inner sheath handle 6805 to advance one or more segments of the outer sheath expansion zone 6875 along the occlusion with perfusion device 1500. The occlusion with perfusion device 1500 is in a stowed condition within the expansion zone 6875 against the outer wall of the guide catheter 6840. The guide catheter 6840 is shown extending from and beyond the stowed occlusion device 1500 and the distal end of the outer sheath expansion zone 6875. This configuration is similar to that illustrated in FIG. 57H.

FIG. 68D is an enlarged view of the final position of the second or outer sheath handle 6810 and the first or the inner sheath handle 6805 at the proximal end. When the handles 6810, 6805 are in this position, the occlusion with perfusion device 1500 is stowed and distal perfusion occurs while a procedure is performed using the guide catheter or other devices inserted via the lumen of the shaft coupled to the occlusion with perfusion device.

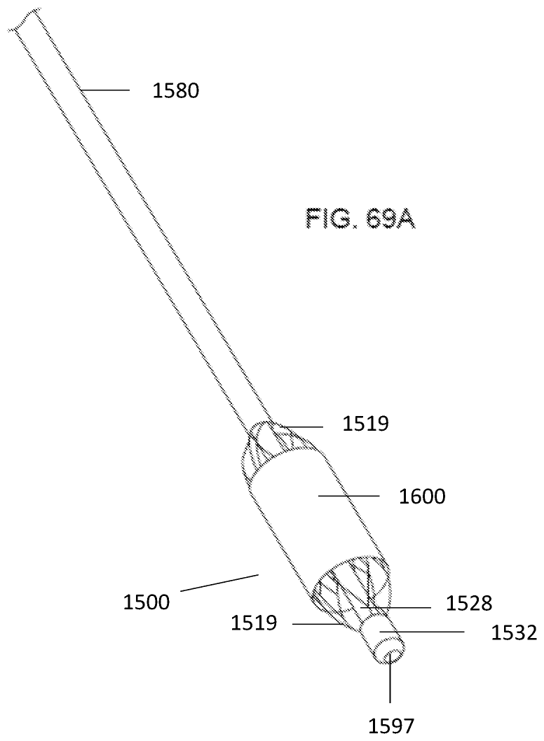

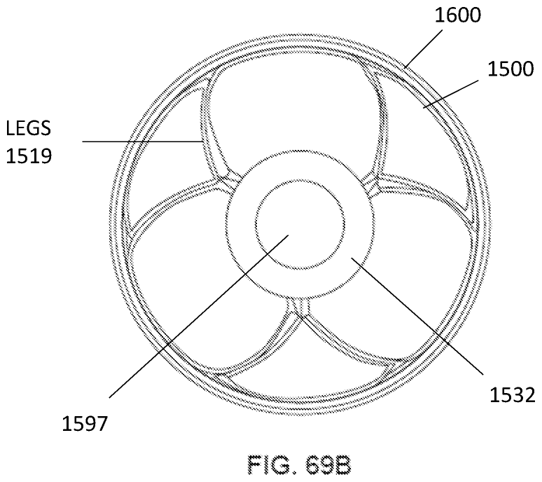

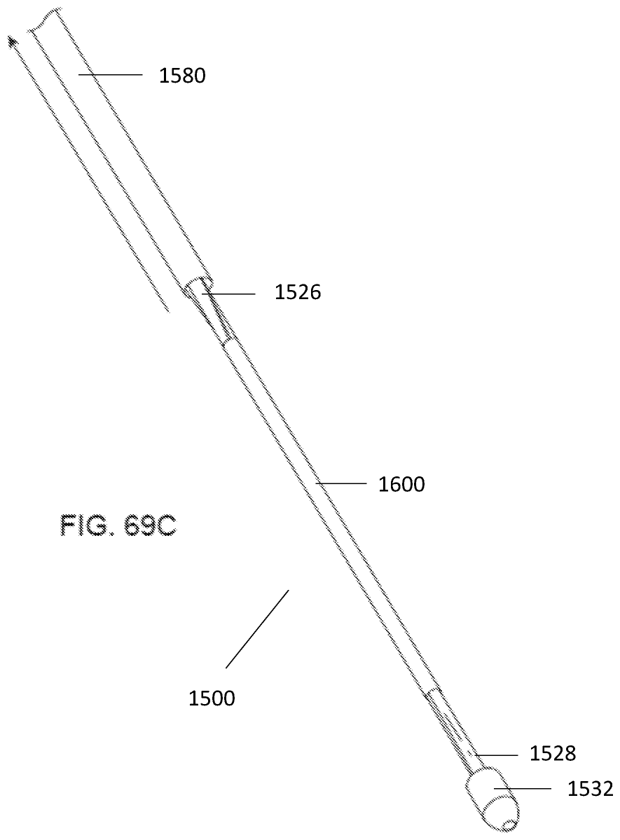

FIG. 69A is a perspective view of a distal end of an additional embodiment of an occlusion with perfusion device 1500 in a deployed configuration. The legs 1519 of proximal end of the scaffold are attached to the distal end of the outer shaft 1580. The legs 1519 at the distal end of the scaffold are attached to the distal end of the inner tube 1528 or to an atraumatic tip 1532. The working channel or lumen 1597 of the inner shaft 1525 is shown in the atraumatic tip 1532.

FIG. 69B is a distal end view of the deployed occlusion with perfusion device of FIG. 69A.

FIG. 69C is a perspective view of the occlusion with perfusion device of FIG. 69A transitioned into a stowed configuration by proximal movement of the outer sheath 1580 as indicated by the arrow. In the stowed configuration the occlusion with perfusion device is against the outer wall of the inner shaft or occlusion device shaft 1525. The outer shaft and inner shaft may be coupled to the handles of FIG. 68D in one embodiment. Relative movement of the handles will transition the occlusion device of FIG. 69A between a stowed condition (FIG. 69C) and a deployed or occlusion position (FIG. 69A).

FIG. 70 a view of a diagrammatic portion of a torso of a patient. The aorta is illustrated from the aortic arch to the internal and external iliac arteries along with many of the branch vessels. Also visible in this view is a portion of the bony anatomy including the vertebra of the spine, the right and the left pelvic bones, the sacrum and a portion of the coccyx.

The ascending aorta arises from the aortic orifice from the left ventricle and ascends to become the aortic arch. It is 2 inches long in length and travels with the pulmonary trunk in the pericardial sheath. The branches include the left and right aortic sinuses are dilations in the ascending aorta, located at the level of the aortic valve. They give rise to the left and right coronary arteries that supply the myocardium.

The aortic arch is a continuation of the ascending aorta and begins at the level of the second sternocostal joint. It arches superiorly, posteriorly and to the left before moving inferiorly. The aortic arch ends at the level of the T4 vertebra. There are three major branches arising from the aortic arch. From proximal to distal they include:

Brachiocephalic trunk: The first and largest branch that ascends laterally to split into the right common carotid and right subclavian arteries. These arteries supply the right side of the head and neck, and the right upper limb.

Left common carotid artery: Supplies the left side of the head and neck.

Left subclavian artery: Supplies the left upper limb.

The thoracic aorta or descending aorta spans from the level of T4 to T12. Continuing from the aortic arch, it initially begins to the left of the vertebral column but approaches the midline as it descends. It leaves the thorax via the aortic hiatus in the diaphragm, and becomes the abdominal aorta. The branches include, in descending order:

Bronchial arteries: Paired visceral branches arising laterally to supply bronchial and peribronchial tissue and visceral pleura. However, most commonly, only the paired left bronchial artery arises directly from the aorta whilst the right branches off usually from the third posterior intercostal artery.

Mediastinal arteries: Small arteries that supply the lymph glands and loose areolar tissue in the posterior mediastinum.

Oesophageal arteries: Unpaired visceral branches arising anteriorly to supply the oesophagus.

Pericardial arteries: Small unpaired arteries that arise anteriorly to supply the dorsal portion of the pericardium.

Superior phrenic arteries: Paired parietal branches that supply the superior portion of the diaphragm.

Intercostal and subcostal arteries: Small paired arteries that branch off throughout the length of the posterior thoracic aorta. The 9 pairs of intercostal arteries supply the intercostal spaces, with the exception of the first and second (they are supplied by a branch from the subclavian artery). The subcostal arteries supply the flat abdominal wall muscles.

The abdominal aorta is a continuation of the thoracic aorta beginning at the level of the T12 vertebrae. It is approximately 13 cm long and ends at the level of the L4 vertebra. At this level, the aorta terminates by bifurcating into the right and left common iliac arteries that supply the lower body. The branches of the abdominal aorta are, in descending order:

Inferior phrenic arteries: Paired parietal arteries arising posteriorly at the level of T12. They supply the diaphragm.

Coeliac artery: A large, unpaired visceral artery arising anteriorly at the level of T12. It is also known as the celiac trunk and supplies the liver, stomach, abdominal oesophagus, spleen, the superior duodenum and the superior pancreas.

Superior mesenteric artery: A large, unpaired visceral artery arising anteriorly, just below the celiac artery. It supplies the distal duodenum, jejuno-ileum, ascending colon and part of the transverse colon. It arises at the lower level of L1.

Middle suprarenal arteries: Small paired visceral arteries that arise either side posteriorly at the level of L1 to supply the adrenal glands.

Renal arteries: Paired visceral arteries that arise laterally at the level between L1 and L2. They supply the kidneys.

Gonadal arteries: Paired visceral arteries that arise laterally at the level of L2. Note that the male gonadal artery is referred to as the testicular artery and in females, the ovarian artery.

Inferior mesenteric artery: A large, unpaired visceral artery that arises anteriorly at the level of L3. It supplies the large intestine from the splenic flexure to the upper part of the rectum.

Median sacral artery: An unpaired parietal artery that arises posteriorly at the level of L4 to supply the coccyx, lumbar vertebrae and the sacrum.

Lumbar arteries: There are four pairs of parietal lumbar arteries that arise posterolaterally between the levels of L1 and L4 to supply the abdominal wall and spinal cord.

In various alternative embodiments, the length of an occlusion with perfusion device may be adapted in order to cover one or more branches of an aorta of a patient. The length of the device used to temporarily occlude branches of the aorta is the therapeutic length of the occlusion with perfusion device. The therapeutic effect of temporarily and reversibly occluding one or more branches of the aorta may be accomplished by the among of scaffold covering material applied to the scaffold of the occlusion with perfusion device. These alternatives are applicable to occlusion with perfusion devices having an occlusion with perfusion device and an outer sheath. These alternatives are also applicable to those embodiments where the occlusion with perfusion device and outer sheath are modified to provide a single vascular access point via the lumen of the inner shaft coupled to the occlusion device for a variety of types and sizes of guide catheters, therapeutic catheters, therapeutic devices, vascular prosthesis, implantable devices including transcatheter aortic valves (See FIGS. 71 and 72) and described elsewhere herein.

In one aspect, an embodiment of an occlusion with perfusion device may be configured into a number of different therapeutic lengths. The different therapeutic lengths advantageously allow different embodiments of the occlusion with perfusion device to provide selective, temporary occlusion of a variety or mixed combination of branches of the aorta. The therapeutic length for a particular occlusion with perfusion device will depend on the clinical scenario in which the device is employed. In one exemplary configuration, an occlusion with perfusion device may be used to selectively, temporarily and reversibly occlude the renal arteries. In another exemplary configuration, an occlusion with perfusion device may be used to selectively, temporarily and reversibly occlude some or all of the aorta branches in the abdominal aorta. Such an occlusion with perfusion device may have a length from a proximal end that is superior to the iliac split and a distal end that is at or inferior to the diaphragm. In still another exemplary configuration, an occlusion with perfusion device may be used to selectively, temporarily and reversibly occlude some or all of the aorta branches in the thoracic and abdominal aorta. Such an occlusion with perfusion device may have a length from a proximal end that is superior to the iliac split and a distal end that is at or inferior to the diaphragm.

Exemplary Therapeutic Length 1

FIG. 70 includes an exemplary occlusion with perfusion device length (1). In one embodiment, the therapeutic length of the occlusion device means that length of the device along a vessel containing the device within which the device may occlude a side branch of the vessel. In the case where the device is intended to selectively, temporarily and reversibly occlude the renal arteries, the length of the device would be about 5.5 cm or in a range of about 4.5 cm to about 6.5 cm. In one possible deployment scenario within the aorta for selective and temporary occlusion of the renal arteries, the distal end of the device is position at or near L1, the first lumbar vertebra. In this position, when the device is deployed into an occlusion with distal profusion configuration, a portion of the covering that is unattached to the scaffold structure will distend into the opening of the branch vessel. In this illustrative example, the branch vessel is a renal artery.

Exemplary Therapeutic Length 2

FIG. 70 includes an exemplary occlusion with perfusion device length (2). In an additional alternative embodiment, the therapeutic length of the occlusion device is selected to occlude the renal arteries as well as the branches of the aorta along the abdominal aorta or the celiac trunk. In one illustrative example, the occlusion with perfusion device would have a length from a proximal end that is superior to the iliac split and a distal end that is at or inferior to the diaphragm. In the case where the device is intended to selectively, temporarily and reversibly occlude the renal arteries and those within a portion of the abdominal aorta, the length of the device would be about 11 cm or in a range of about 10 cm to about 12 cm. In one possible deployment scenario within the aorta for selective and temporary occlusion of the renal arteries and branches of the abdominal aorta, the distal end of the device is position at or near vertebra T9, or at or near the diaphragm or at or near the abdominal hiatus or within the celiac trunk. In this position, a device with this therapeutic length when the device is deployed into an occlusion with distal profusion configuration, a portion of the covering that is unattached to the scaffold structure will distend into the opening of the renal arteries and one or more of the branch vessels of the abdominal aorta and/or the celiac trunk or branch vessels inferior to the renal arteries and/or branch vessels superior to the renal arteries. Still further, in various alternative embodiments, the branch vessels that may be temporarily occluded by the device include, for example and not limitation, the inferior mesenteric artery, the superior mesenteric artery, the gonadal artery, the common hepatic artery, the adrenal artery, the left and right gastric arteries, and the splenic artery.

Exemplary Therapeutic Length 3

FIG. 70 includes an exemplary occlusion with perfusion device length (3). In an additional alternative embodiment, the therapeutic length of the occlusion device is selected to occlude the renal arteries as well as the branches of the aorta along the abdominal aorta or the celiac trunk (Length 2) and an additional therapeutic length of the device which could be used to occlude branches of the thoracic aorta.

In one illustrative example, the occlusion with perfusion device would have a length from a proximal end that is superior to the iliac split and a distal end that is at or superior to the diaphragm. In the case where the device is intended to selectively, temporarily and reversibly occlude the renal arteries and those within a portion of both the abdominal aorta and the thoracic aorta, the length of the device would be about 17 cm or in a range of about 15 cm to about 19 cm. In one possible deployment scenario within the aorta for selective and temporary occlusion of the renal arteries and branches of the abdominal aorta, the celiac trunk and the thoracic aorta, the distal end of the device is position at or near vertebra T6. In this position, a device with this therapeutic length when the device is deployed into an occlusion with distal profusion configuration, a portion of the covering that is unattached to the scaffold structure will distend into the opening of the renal arteries and one or more of the branch vessels of the abdominal aorta and/or the celiac trunk and/or branch vessels of the thoracic aorta. In addition to the branch vessels mentioned above, the device may also be used to selectively and temporarily occlude one or more of the branch vessels superior to the aortic hiatus and inferior to the aortic arch.

In each of these various therapeutic lengths, it is to be appreciated that the amount or percentage of the scaffold device that is covered, the location of the covering on the device relative to a likely branch vessel location when deployed as well as the relative size and number of branch openings are each one different design attributes for various alternatives. In still other alternatives, the device may be covered along the entire length and deployed in a general way as an emergency occlusion device when a length of the aorta or branch vessel has been damaged. In this way, a device of therapeutic length 1 may be used but instead of directing delivery to the renal arteries, delivery is guided to the injured or suspected injured branch vessel or portion of the aorta. In a similar way, therapeutic lengths 2 and 3 may be similarly employed should the zone of damage to the aorta or the number of branches involved require temporary occlusion in support of repairs to the damaged aorta or damaged branch vessel or vessels.

Additionally or alternatively, the different lengths of the device may be employed to protect other organs or portions of the body from harmful materials similar to the way that the temporary occlusion of the renal arteries during contrast injection aids in preventing harm to the kidneys. As such, by temporarily occluding one or more branch vessels of the abdominal aorta, celiac trunk or thoracic aorta, reduced exposure of those organs or body functions supported by the aorta may also be provided. In still other alternative clinical scenarios, a patient who is undergoing chemotherapy may also benefit from the use of an embodiment of a temporary occlusion device to prevent chemotherapeutic agents from being carried into the organs and functions supplied by the abdominal aorta, celiac trunk or thoracic aorta.

In still other aspects, an occlusion with perfusion device may also have sections where the cover material or configuration may vary depending upon the clinical scenario and the grouping of branch vessels to be temporarily and reversibly occluded. As a result, there are also provided occlusion with perfusion device embodiments having one or more or a combination of: continuous scaffold sections that are uncovered, discontinuous scaffold covering sections, scaffold coverings including pressure relief features (FIGS. 33B, 40A-40E), scaffold covering both sides or only one side of a scaffold, scaffold covering sections of different, partial or tapered shapes (FIGS. 12C, 14D, 34, 40F insert figs for scalloped covers), different sectional configurations (FIGS. 30, 31, 32, 33A, 36, 37) as well as those scaffold covering configurations having attached and unattached sections (FIGS. 29A-29C, 40A-40E, 44B and 44C).

FIG. 71 is a table detailing the characteristics and other details of a number of different devices for transcatheter aortic valve replacement (TAVR) procedures. FIG. 72 is a table detailing the various exemplary sizes of introducers and sheaths used in the delivery of a variety of different sized TAVR devices. These and other details were obtained from “Current TAVR devices” by Denise Todaro, M D et al obtained online from Cardiac Interventions Today, March/April 2017 edition available at: https://citoday.com/articles/2017-mar-apr/current-tavr-devices.

In embodiments of the single point vascular access device adapted for combined use with TAVR delivery, the outer sheath and the occlusion with perfusion device and associated guide catheter are adapted to accommodate the size of the particular device being implanted. As described elsewhere, the occlusion with perfusion device operates to protect the kidneys from damage by temporarily occluding the renal arteries when contrast is used. Other organs may also be protected from potential damage from contrast exposure by selecting an occlusion with perfusion device of an appropriate therapeutic length for those organs to be protected.

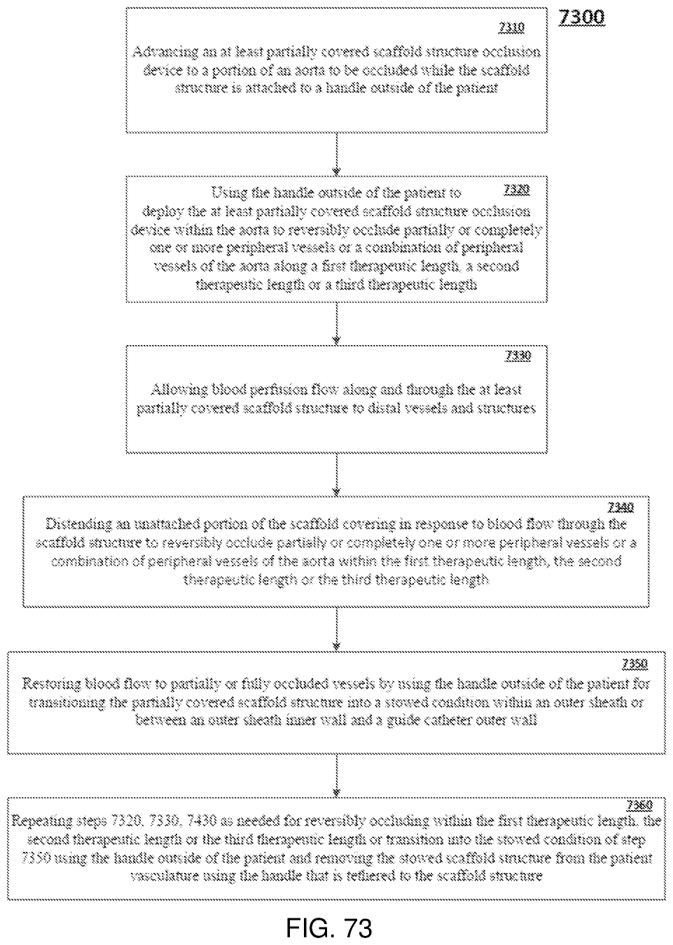

FIG. 73 is a flow chart of an exemplary method of providing occlusion with perfusion using an embodiment of a vascular occlusion device according to the method 7300.

First, at step 7310, there is the step of advancing an at least partially covered scaffold structure occlusion device to a portion of an aorta to be occluded while the scaffold structure is attached to a handle outside of the patient. This step may be further understood by reference to FIG. 55 and FIG. 70.

Next, at step 7320, there is the step of using the handle outside of the patient to deploy the at least partially covered scaffold structure occlusion device within the aorta to reversibly occlude partially or completely one or more peripheral vessels or a combination of peripheral vessels of the aorta along a first therapeutic length, a second therapeutic length or a third therapeutic length. This step is performed by appropriate manipulation of a handle embodiment described herein according to the disclosure related to FIG. 70.

Next, at step 7330, there is the step of allowing blood perfusion flow through the at least partially covered scaffold structure to distal vessels and structures.

Next, at step 7340, there is a step of distending an unattached portion of the scaffold covering in response to blood flow through the scaffold structure to reversibly occlude partially or completely one or more peripheral vessels or a combination of peripheral vessels of the aorta within the first therapeutic length, the second therapeutic length or the third therapeutic length. This step may be appreciated by way of illustration and not limitation the additional details of FIGS. 44C and 51C.

Next, at step 7350, there is a step of restoring blood flow to partially or fully occluded vessels by using the handle outside of the patient for transitioning the partially covered scaffold structure into a stowed condition within an outer sheath or between an outer sheath inner wall and a guide catheter outer wall. Aspects of the details of this step may be appreciated by reference to FIGS. 39A, 51A and 57H, for example.

Next, at step 7360, there is a step of repeating steps 7320, 7330, 7430 as needed for reversibly occluding within the first therapeutic length, the second therapeutic length or the third therapeutic length or transition into the stowed condition of step 7350 using the handle outside of the patient and removing the stowed scaffold structure from the patient vasculature using the handle that is tethered to the scaffold structure.

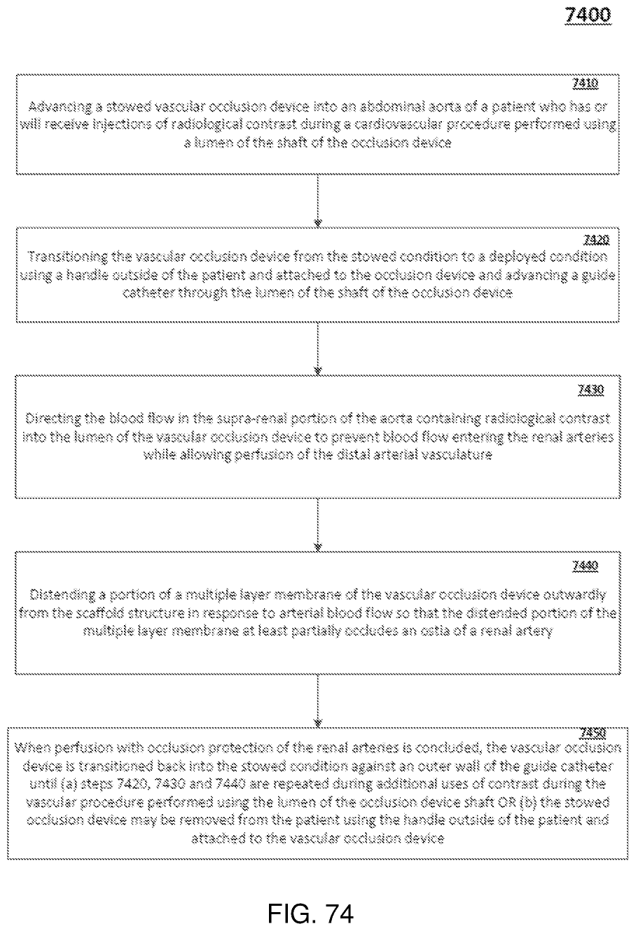

FIG. 74 is a flow chart of an exemplary method of providing occlusion with perfusion using an embodiment of a vascular occlusion device according to the method 7400.

First, at step 7410 there is a step of advancing a stowed vascular occlusion device into an abdominal aorta of a patient who has or will receive injections of radiological contrast during a cardiovascular procedure performed using a lumen of the shaft of the occlusion device. This step may be further understood by reference to FIG. 55 and FIG. 70.

Next, at step 7420, there is a step of transitioning the vascular occlusion device from the stowed condition to a deployed condition using a handle outside of the patient and attached to the occlusion device and advancing a guide catheter through the lumen of the shaft of the occlusion device. This step may be further understood with reference to FIGS. 51A, 51B, and 57F.

Next, at step 7430, there is a step of directing the blood flow in the supra-renal portion of the aorta containing radiological contrast into the lumen of the vascular occlusion device to prevent blood flow entering the renal arteries while allowing perfusion of the distal arterial vasculature. This step may be further understood with reference to FIGS. 44A-44C.

Next, at step 7440, there is a step distending a portion of a multiple layer membrane of the vascular occlusion device outwardly from the scaffold structure in response to arterial blood flow so that the distended portion of the multiple layer membrane at least partially occludes an ostia of a renal artery. This step may be further understood with reference to FIGS. 44A-44C.

Next, at step 7450, when perfusion with occlusion protection of the renal arteries is concluded, the vascular occlusion device is transitioned back into the stowed condition against an outer wall of the guide catheter until (a) steps 7420, 7430 and 7440 are repeated during additional uses of contrast during the vascular procedure performed using the lumen of the occlusion device shaft OR (b) the stowed occlusion device may be removed from the patient using the handle outside of the patient and attached to the vascular occlusion device. This step may be further understood by reference to FIGS. 51A and 57H.

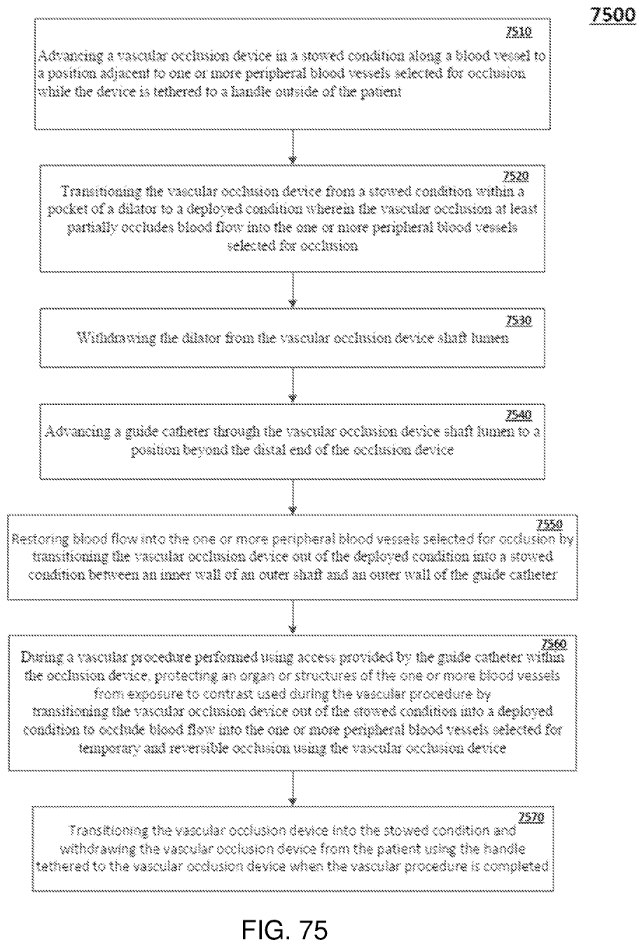

FIG. 75 is a flow chart of an exemplary method of providing occlusion with perfusion using an embodiment of a vascular occlusion device according to the method 7500.

First, at step 7510, there is the step of advancing a vascular occlusion device in a stowed condition along a blood vessel to a position adjacent to one or more peripheral blood vessels selected for occlusion while the device is tethered to a handle outside of the patient. This step may be further understood by reference to FIG. 55 and FIG. 70.

Next, at step 7520, there is the step of transitioning the vascular occlusion device from a stowed condition within a pocket of a dilator to a deployed condition wherein the vascular occlusion at least partially occludes blood flow into the one or more peripheral blood vessels selected for occlusion. This step may be further appreciated by reference to FIGS. 57A-59.

Next, at step 7530, there is the step of withdrawing the dilator from the vascular occlusion device shaft lumen. This step may be appreciated by specific reference to FIGS. 57D and 57E.

Next, at step 7540, there is the step of advancing a guide catheter through the vascular occlusion device shaft lumen to a position beyond the distal end of the occlusion device. This step may be appreciated by reference to FIG. 57F.

Next, at step 7550, there is the step of restoring blood flow into the one or more peripheral blood vessels selected for occlusion by transitioning the vascular occlusion device out of the deployed condition into a stowed condition between an inner wall of an outer shaft and an outer wall of the guide catheter. This step may be appreciated by reference to FIGS. 57H, 51A, 67C and 68C.

Next, at step 7560, during a vascular procedure performed using access provided by the guide catheter within the occlusion device, protecting an organ or structures of the one or more blood vessels from exposure to contrast used during the vascular procedure by transitioning the vascular occlusion device out of the stowed condition into a deployed condition to occlude blood flow into the one or more peripheral blood vessels selected for temporary and reversible occlusion using the vascular occlusion device. The benefits of this step and the protection of various organs and structures may be appreciated with reference to FIG. 70.

Finally, at step 7570, there is a step of transitioning the vascular occlusion device into the stowed condition and withdrawing the vascular occlusion device from the patient using the handle tethered to the vascular occlusion device when the vascular procedure is completed.

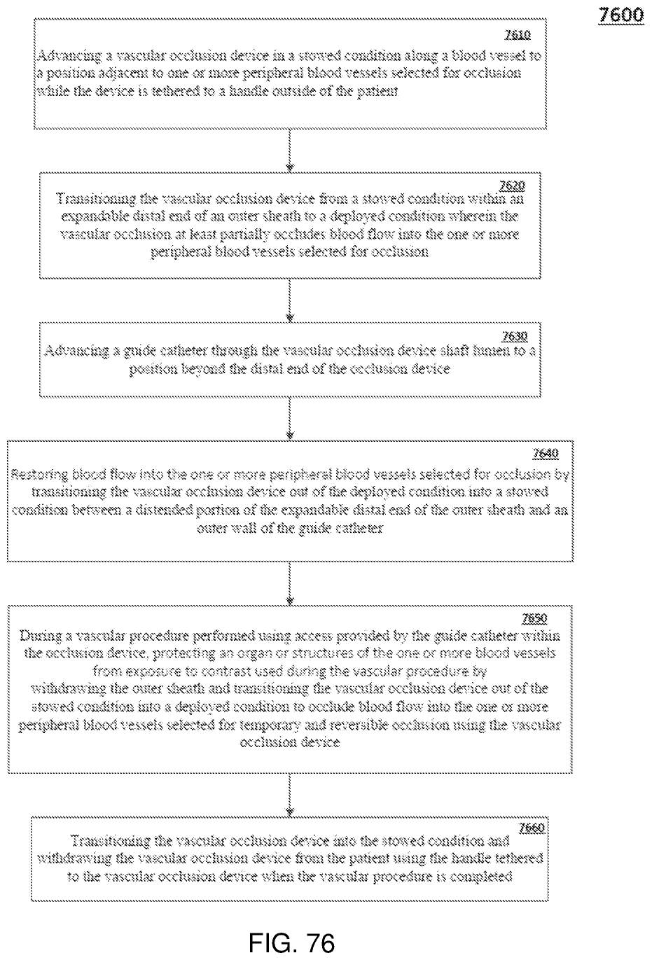

FIG. 76 is a flow chart of an exemplary method of providing occlusion with perfusion using an embodiment of a vascular occlusion device according to the method 7600.

First, at step 7610, there is the step of advancing a vascular occlusion device in a stowed condition along a blood vessel to a position adjacent to one or more peripheral blood vessels selected for occlusion while the device is tethered to a handle outside of the patient.

Next, at step 7620, there is the step of transitioning the vascular occlusion device from a stowed condition within an expandable distal end of an outer sheath to a deployed condition wherein the vascular occlusion at least partially occludes blood flow into the one or more peripheral blood vessels selected for occlusion. Exemplary embodiments related to the expandable section of an outer sheath include, by way of example and not limitation, those described with regard to FIGS. 60-68C.

Next, at step 7630, there is the step of advancing a guide catheter through the vascular occlusion device shaft lumen to a position beyond the distal end of the occlusion device. This step may be appreciated by reference to FIGS. 68A and 57F.

Next, at step 7640, there is the step of restoring blood flow into the one or more peripheral blood vessels selected for occlusion by transitioning the vascular occlusion device out of the deployed condition into a stowed condition between a distended portion of the expandable distal end of the outer sheath and an outer wall of the guide catheter. This step may be appreciated with reference, for example, to FIGS. 67A-67E and 68C.

Next, at step 7650, during a vascular procedure performed using access provided by the guide catheter within the occlusion device, protecting an organ or structures of the one or more blood vessels from exposure to contrast used during the vascular procedure by withdrawing the outer sheath and transitioning the vascular occlusion device out of the stowed condition into a deployed condition to occlude blood flow into the one or more peripheral blood vessels selected for temporary and reversible occlusion using the vascular occlusion device. This step may be accomplished by reversing the direction of movement in FIGS. 67A-67C.

Finally, at step 7660, there is a step of transitioning the vascular occlusion device into the stowed condition and withdrawing the vascular occlusion device from the patient using the handle tethered to the vascular occlusion device when the vascular procedure is completed.

Exemplary Combination Vascular Access and Occlusion with Perfusion Devices

The various alternative configurations and capabilities of the perfusion with occlusion device and combination occlusion with access device may be sized for a variety of applications and different vascular procedures. In one aspect, for example, sizes ranging from 5 Fr to 8 Fr (0.065 to 0.105 inches) when used for occlusion with perfusion alone and from 6 Fr to 24 Fr (0.079-0.315 inches) when used as a combination occlusion with perfusion and vascular access device. Additionally, the occlusion device shaft lumen may also be sized based on when used alone or in a combination product for vascular access. When used alone, the lumen of the occlusion device shaft may range from 4 Fr to 7 Fr (0.053 to 0.092 inches) When used in a combination occlusion and vascular access product the lumen size will be increased to allow access to a range of different sized guide catheters. In this case, the lumen will range from 5 Fr to 22 Fr (0.066-0.288 inches)

Advantageously, a dilator may also be used which has been modified to provide a pocket sized to hold an occlusion with perfusion device in a stowed configuration to further decrease the profile—size of the combination device during introduction to the vasculature. The dilator pocket may have a length of about 10 cm, or a range of 5 cm to 40 cm, for the recessed portion of the dilator used to hold the occlusion device. The recessed outer diameter will be about 0.035 inches, with a range of 0.035 to 0.050 inches. The recessed portions inner diameter will be about 0.021 inches, with a range of 0.021 inches and 0.040 inches. (See FIGS. 57A-59).

Various exemplary embodiments of the invention are described herein. Reference is made to these examples in a non-limiting sense. They are provided to illustrate more broadly applicable aspects of the invention. Various changes may be made to the invention described and equivalents may be substituted without departing from the true spirit and scope of the invention. In addition, many modifications may be made to adapt a particular situation, material, composition of matter, process, process act(s) or step(s) to the objective(s), spirit or scope of the present invention. Further, as will be appreciated by those with skill in the art that each of the individual variations described and illustrated herein has discrete components and features which may be readily separated from or combined with the features of any of the other several embodiments without departing from the scope or spirit of the present inventions. All such modifications are intended to be within the scope of claims associated with this disclosure.

Any of the devices described for carrying out the subject diagnostic or interventional procedures may be provided in packaged combination for use in executing such interventions. These supply “kits” may further include instructions for use and be packaged in sterile trays or containers as commonly employed for such purposes.

The invention includes methods that may be performed using the subject devices. The methods may comprise the act of providing such a suitable device. Such provision may be performed by the end user. In other words, the “providing” act merely requires the end user obtain, access, approach, position, set-up, activate, power-up or otherwise act to provide the requisite device in the subject method. Methods recited herein may be carried out in any order of the recited events which is logically possible, as well as in the recited order of events.

Exemplary aspects of the invention, together with details regarding material selection and manufacture have been set forth above. As for other details of the present invention, these may be appreciated in connection with the above-referenced patents and publications as well as generally known or appreciated by those with skill in the art. For example, one with skill in the art will appreciate that one or more lubricious coatings (e.g., hydrophilic polymers such as polyvinylpyrrolidone-based compositions, fluoropolymers such as tetrafluoroethylene, hydrophilic gel or silicones) may be used in connection with various portions of the devices, such as relatively large interfacial surfaces of movably coupled parts, if desired, for example, to facilitate low friction manipulation or advancement of such objects relative to other portions of the instrumentation or nearby tissue structures. The same may hold true with respect to method-based aspects of the invention in terms of additional acts as commonly or logically employed.

In addition, though the invention has been described in reference to several examples optionally incorporating various features, the invention is not to be limited to that which is described or indicated as contemplated with respect to each variation of the invention. Various changes may be made to the invention described and equivalents (whether recited herein or not included for the sake of some brevity) may be substituted without departing from the true spirit and scope of the invention. In addition, where a range of values is provided, it is understood that every intervening value, between the upper and lower limit of that range and any other stated or intervening value in that stated range, is encompassed within the invention.

Also, it is contemplated that any optional feature of the inventive variations described may be set forth and claimed independently, or in combination with any one or more of the features described herein. Reference to a singular item, includes the possibility that there are plural of the same items present. More specifically, as used herein and in claims associated hereto, the singular forms “a,” “an,” “said,” and “the” include plural referents unless the specifically stated otherwise. In other words, use of the articles allow for “at least one” of the subject item in the description above as well as claims associated with this disclosure. It is further noted that such claims may be drafted to exclude any optional element. As such, this statement is intended to serve as antecedent basis for use of such exclusive terminology as “solely,” “only” and the like in connection with the recitation of claim elements, or use of a “negative” limitation.

Without the use of such exclusive terminology, the term “comprising” in claims associated with this disclosure shall allow for the inclusion of any additional element—irrespective of whether a given number of elements are enumerated in such claims, or the addition of a feature could be regarded as transforming the nature of an element set forth in such claims. Except as specifically defined herein, all technical and scientific terms used herein are to be given as broad a commonly understood meaning as possible while maintaining claim validity.

The breadth of the present invention is not to be limited to the examples provided and/or the subject specification, but rather only by the scope of claim language associated with this disclosure.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

Although preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein can be employed in practicing the invention. It is intended that the following claims define the scope of the invention and that methods and structures within the scope of these claims and their equivalents be covered thereby.

What is claimed is:

1. A vascular occlusion device, comprising:
a. A handle having a first part and a second part;
b. An inner shaft coupled to the handle first part;
c. An outer shaft over the inner shaft and coupled to the handle second part;
d. A scaffold structure having a distal end, a scaffold transition zone and a proximal end having one or a plurality of legs wherein the one leg or each leg of the plurality legs is coupled to a distal portion of the inner shaft, wherein the scaffold structure moves from a stowed configuration when the outer shaft is extended over the scaffold structure and a deployed configuration when the outer shaft is retracted from covering the scaffold structure by relative movement of the handle first part and the handle second part; and
e. A one or more layer scaffold covering over at least a portion of the scaffold structure, the one or more layer scaffold covering having a distal scaffold attachment zone where a portion of the scaffold covering is attached to a distal portion of the scaffold, a proximal scaffold attachment zone where a portion of the scaffold covering is attached to a proximal portion of the scaffold and an unattached zone between the distal attachment zone and the proximal attachment zone wherein the scaffold covering is unattached to an adjacent portion of the scaffold.

2. The vascular occlusion device of claim 1 wherein the plurality of legs is two legs, three legs, four legs or more legs.

3. The vascular occlusion device of claim 1 or claim 2 wherein the scaffold covering extends from the distal end of the scaffold structure to the one leg or to each of the two legs, three legs, the four legs or the more legs.

4. The vascular occlusion device of claim 1 wherein the scaffold covering extends from the distal end of the scaffold structure proximally to cover approximately 20%, 50%, 80% or 100% of the overall length of the scaffold structure.

5. The vascular occlusion device of claim 1 wherein the scaffold covering extends completely circumferentially about the scaffold structure from the distal attachment zone to the proximal attachment zone.

6. The vascular occlusion device of claim 1 the scaffold covering further comprising one or more pressure relief features within the scaffold covering.

7. The vascular occlusion device of claim 6 wherein the one or more pressure relief features is a slit or an opening in the scaffold covering.

8. The vascular occlusion device of claim 1 wherein a distal end portion of the outer sheath further comprises an expansion zone.

9. The vascular occlusion device of claim 8 wherein the expansion zone of the outer sheath comprises a plurality of sections joined by one or more flexible couplings.

10. The vascular occlusion device of claim 9 wherein each section of the plurality of sections includes two or three segments.

11. The vascular occlusion device of claim 8 wherein when outer sheath is advanced over the scaffold structure in a deployed configuration, the expansion zone of the outer sheath transitions into a larger diameter to accommodate the scaffold structure of the occlusion with perfusion device.

12. The vascular occlusion device of claim 1 wherein a distal end portion of the outer sheath further comprises an expansion zone having one or a combination of slits, zig-zag cuts, braids or expansion features.

13. A combination vascular occlusion and vascular access device, comprising:
a. A handle;
b. An inner shaft coupled to the handle, the inner shaft having a lumen accessible via a hemostasis valve in the handle;
c. An outer shaft over the inner shaft and coupled to the handle;
d. An occlusion with perfusion device having a scaffold structure coupled to the inner shaft, and a scaffold covering over at least a portion of the scaffold structure, the scaffold covering having a distal scaffold attachment zone where a portion of the scaffold covering is attached to a distal portion of the scaffold, a proximal scaffold attachment zone where a portion of the scaffold covering is attached to a proximal portion of the scaffold and an unattached zone between the distal attachment zone and the proximal attachment zone wherein the scaffold covering is unattached to an adjacent portion of the scaffold; and
e. A dilator having an occlusion device pocket proximal to a distal end of the dilator, the occlusion device pocket is sized to hold the occlusion with perfusion device.

14. The device of claim 13 wherein the occlusion device pocket is formed by dilator shaft that joins the dilator tip to the dilator body.

15. The device of claim 13 wherein the length of the occlusion device pocket is 5 cm, 10 cm, 20 cm or 40 cm.

16. The device of claim 15 wherein the occlusion device pocket has a recessed outer diameter of about 0.035 inches or from 0.035 to 0.050 inches and a recessed portion inner diameter of about 0.021 inches or from 0.021 inches to 0.040 inches.

17. The device of claim 13 the scaffold structure having a distal end, a scaffold transition zone and a proximal end having one or a plurality of legs wherein the one leg or each leg of the plurality legs is coupled to a distal portion of the inner shaft, wherein the scaffold structure moves from a stowed configuration when the outer shaft is extended over the scaffold structure and a deployed configuration when the outer shaft is retracted from covering the scaffold structure.

18. The device of claim 13 the lumen of the inner shaft sized to allow access for a guide catheter adapted for passing an intravascular device that is one of a diagnostic instrument, or an instrument selected from the group consisting of: an angiography catheter, an intravascular ultrasound testing instrument, or an intravascular optical coherence tomography instrument, and the therapeutic instrument is preferably a balloon catheter, a drug-eluting balloon catheter, a bare metal stent, a drug-eluting stent, a drug-eluting biodegradable stent, a rotablator, a thrombus suction catheter, a drug administration catheter, a guiding catheter, a support catheter, or a device or a prosthesis delivered as part of a TAVR, TMVR, or TTVR procedure or system.

19. The device of claim 13 wherein the scaffold covering extends partially circumferentially about the scaffold structure from the distal attachment zone to the proximal attachment zone with an uncovered scaffold structure.

20. The device of claim 13 occlusion device of claim 6 wherein the scaffold covering extends partially circumferentially about 270 degrees of the scaffold structure from the distal attachment zone to the proximal attachment zone.

21. The device of claim 13 wherein a first scaffold covering extends partially circumferentially about 45 degrees of the scaffold structure from the distal attachment zone to the proximal attachment zone and a second scaffold covering extends partially circumferentially about 45 degrees of the scaffold structure from the distal attachment zone to the proximal attachment zone, wherein the first scaffold covering and the second scaffold covering are on opposite sides of the longitudinal axis of the scaffold structure.

22. The device of claim 13 wherein the scaffold structure is formed from slots cut into a tube or a plurality of formed wires, or a single wire form.

23. The device of claim 13 wherein scaffold covering is formed from a single or multiple layers.

24. The device of claim 23 wherein the layers of the multiple layer scaffold covering are selected from ePFTE, PTFE, FEP, polyurethane or silicone.

25. The vascular occlusion device of claim 1 or claim 13 wherein the scaffold covering or the more than one layers of a multiple layer scaffold covering is applied to a scaffold structure external surface, to a scaffold structure internal surface, to encapsulate the distal scaffold attachment zone and the proximal scaffold attachment zone, as a series of spray coats, dip coats or electron spin coatings to the scaffold structure.

26. The vascular occlusion device of claim 1 or claim 13 wherein the multiple layer scaffold covering has a thickness of 5-100 microns.

27. The vascular occlusion device of claim 1 or claim 13 wherein the multiple layer scaffold covering has a thickness of about 0.001 inches in an unattached zone and a thickness of about 0.002 inches in an attached zone.

28. A method of providing selective occlusion with distal perfusion using a vascular occlusion device, comprising:
advancing the vascular occlusion device in a stowed condition along a blood vessel to a position adjacent to one or more peripheral blood vessels in the portion of the vasculature of the patient selected for occlusion while the vascular occlusion device is tethered to a handle outside of the patient;
transitioning the vascular occlusion device from the stowed condition to a deployed condition using the handle wherein the vascular occlusion device at least partially occludes blood flow into the one or more peripheral blood vessels selected for occlusion wherein the position of the vascular occlusion device engages with a superior aspect of the vasculature to direct blood flow into and along a lumen defined by a covered scaffold structure of the vascular occlusion device;
deflecting a portion of an unattached zone of the covered scaffold in response to the blood flow through the lumen of the covered scaffold into an adjacent opening of the one or more peripheral blood vessels in the portion of the vasculature of the patient selected for occlusion;
transitioning the vascular occlusion device from the deployed condition to the stowed condition using the handle; and
withdrawing the vascular occlusion device in the stowed condition from the patient.

29. The method of claim 28 wherein the one or more peripheral blood vessels in the portion of the vasculature of the patient selected for occlusion is selected from the group consisting of a hepatic artery, a gastric artery, a celiac trunk, a splenic artery, an adrenal artery, a renal artery, a superior mesenteric artery, an ileocolic artery, a gonadal artery and an inferior mesenteric artery.

30. The method of claim 28 the covered scaffold unattached zone further comprising a position of a portion of the unattached zone to deflect into a portion of at least one of a hepatic artery, a gastric artery, a celiac trunk, a splenic artery, an adrenal artery, a renal artery, a superior mesenteric artery, an ileocolic artery, a gonadal artery and an inferior mesenteric artery when the vascular occlusion device is positioned within a portion of the aorta.

31. A method of temporarily occluding a blood vessel, comprising:
a. Advancing a vascular occlusion device in a stowed condition along a blood vessel to a position adjacent to one or more peripheral blood vessels selected for temporary occlusion;
b. Transitioning the vascular occlusion device from the stowed condition to a deployed condition wherein the vascular occlusion at least partially occludes blood flow into the one or more peripheral blood vessels selected for temporary occlusion while directing the blood flow through and along a lumen of a covered scaffold of the vascular occlusion device; and
c. Transitioning the vascular occlusion device out of the deployed condition to restore blood flow into the one or more peripheral blood vessels selected for temporary occlusion when a period of temporary occlusion is elapsed.

32. The method of claim 31 wherein directing the blood flow through and along the lumen of the vascular occlusion device maintains blood flow to components and vessels distal to the vascular occlusion device while at least partially occluding the blood flow to the one or more peripheral blood vessels.

33. The method of claim 31 or claim 32 wherein the one or more peripheral blood vessels are the vasculature of a liver, a kidney, a stomach, a spleen, an intestine, a stomach, an esophagus, or a gonad.

34. The method of claim 31 or claim 32 wherein the blood vessel is an aorta and the peripheral blood vessels are one or more or a combination of: a hepatic artery, a gastric artery, a celiac trunk, a splenic artery, an adrenal artery, a renal artery, a superior mesenteric artery, an ileocolic artery, a gonadal artery and an inferior mesenteric artery.

35. A method of providing vascular access and reversibly and temporarily occluding a blood vessel, comprising:
a. Advancing an at least partially covered scaffold structure of a tethered vascular occlusion device to a portion of an aorta to be occluded; and
b. Using a handle of the vascular occlusion device to deploy the at least partially covered scaffold structure within the aorta to occlude partially or completely one or more or a combination of: a hepatic artery, a gastric artery, a celiac trunk, a splenic artery, an adrenal artery, a renal artery, a superior mesenteric artery, an ileocolic artery, a gonadal artery and an inferior mesenteric artery using a portion of a multiple layer scaffold covering while simultaneously allowing perfusion flow through a lumen of the at least partially covered scaffold structure to distal vessels and structures; and
c. Using the handle to transition the at least partially covered scaffold structure to a stowed condition between an inner wall of an introducer sheath and an outer wall of a guide catheter within the vascular occlusion device.

36. The method of claim 35 wherein the insertion of the vascular occlusion device or of the at least partially covered scaffold device to a blood vessel which is the aorta is introduced by transfemoral artery approach or by trans-brachial artery approach or by trans-radial artery approach.

37. The method of claim 35 or 36 further comprising: advancing the vascular occlusion device over a guidewire into a position adjacent to a landmark of the skeletal anatomy.

38. A method of providing vascular occlusion with distal perfusion during an interventional vascular procedure, comprising:
Accessing an artery of the arterial vasculature with an introducer sheath having an outer wall and an inner wall and a central lumen which is concentric and coaxial with an occlusion with perfusion device in a stowed condition against the introducer sheath inner wall;
Advancing the introducer sheath with the stowed occlusion with perfusion device into an occlusion position within an aorta with the occlusion with perfusion device adjacent to one or more branch vessels and a distal end of the introducer sheath superior to the one or more branch vessels;
Withdrawing the introducer sheath to transition the occlusion with perfusion device into a deployed condition within the aorta and in position to reversibly occlude the one or more branch vessels;
Advancing a guide catheter through a lumen of a shaft of the occlusion with perfusion device;
Accessing the vasculature with an interventional therapy device via the guide catheter; and
Performing a catheter based therapy at a vascular access therapy site more than 2 cm distal to the occlusion with perfusion device.

39. The method of claim 38 further comprising transitioning the occlusion with perfusion device to a stowed configuration between the inner wall of the introducer sheath and an outer wall of the guide catheter.

40. The method of claim 38 further comprising withdrawing a dilator from the lumen of the occlusion with perfusion shaft before performing the step of advancing a guide catheter through a lumen of a shaft of the occlusion with perfusion device.

41. The method of claim 38 wherein during the step of withdrawing the introducer sheath to transition the occlusion with perfusion device into a deployed condition the occlusion with perfusion device moves out of contact with an occlusion device pocket of a dilator within the lumen of the shaft of the occlusion with perfusion device.

42. The method of claim 38 further comprising transitioning the occlusion with perfusion device to a deployed condition to temporarily and reversibly occlude the one or more branch vessels before performing a step of injecting a contrast solution in support of the catheter based therapy.

43. The method of claim 38 further comprising transitioning the occlusion with perfusion device from the stowed condition in contact with the outer wall of the guide catheter into a position to at least partially occlude at least one ostia of a renal artery and back to the stowed condition at least once during the step of performing a catheter based therapy.

44. The method of claim 38 wherein the catheter based therapy site is at least 8 cm, 10 cm, 20 cm or more distal to the one or more renal ostia.

45. The method of claim 38 wherein the catheter based therapy site is at least 8 cm, 10 cm, 20 cm or more distal to the location of the occlusion with perfusion device.

46. The method of claim 38 wherein the catheter based therapy device is a prosthetic heart valve or component used as part of a TAVR, TMVR or TTVR procedure or system.

47. The method of claim 38 wherein the outer diameter of the introducer sheath is from 7 Fr to 21 Fr.

48. The method of claim 38 wherein after performing the catheter based therapy the catheter based therapy device has a diameter of 15-31 mm.

49. The method of claim 38 the step of performing a catheter based therapy further comprising injecting an amount of contrast agent into the vasculature of the patient.

50. The method of claim 38 further comprising transitioning the occlusion with perfusion device from the stowed condition into a position to at least partially occlude at least one ostia of a renal artery for a contrast protection time period and when the contract protection time period has elapsed transitioning the occlusion with perfusion device back to the stowed condition.

51. The method of claim 38 wherein after the completing of the performing a catheter based therapy and withdrawing all instruments used in the therapy, withdrawing the introducer and occlusion with perfusion device from the artery.

52. The method of claim 50 wherein the step of transitioning the occlusion with perfusion device between stowed and the position to at least partially occlude one or more ostia of the renal arteries is performed without adjusting position or introducer or interfering with the working channel used for the distal cardiovascular procedure.

53. A vascular occlusion device, comprising:
a. A handle having a slider knob;
b. An inner shaft coupled to the handle;
c. An outer shaft over the inner shaft and coupled within the handle to the slider knob;
d. A scaffold structure having at least two legs and a multiple layer scaffold covering, the at least two legs of the scaffold structure attached to an inner shaft coupler in a distal portion of the inner shaft;
e. The multiple layer scaffold covering positioned over at least a portion of the scaffold structure, wherein the scaffold structure moves from a stowed condition when the outer shaft is extended over the scaffold structure and a deployed condition when the outer shaft is retracted from covering the scaffold structure.

54. The vascular occlusion device of claim 53 wherein the scaffold structure is formed from slots cut into a tube.

55. The vascular occlusion device of claim 53 wherein the covering is applied to nearly all, 80%, 70%, 60%, 50%, 30% or 20% of the scaffold structure.

56. The vascular occlusion device of claim 53 wherein the multiple layer scaffold covering is made of ePFTE, PTFE, polyurethane, FEP or silicone.

57. The vascular occlusion device of any one of claim 53-56 wherein the multiple layer scaffold covering is folded over a proximal portion and a distal portion of the scaffold.

58. The vascular occlusion device of any one of claims 53-56 wherein after the multiple layer scaffold covering is attached to the scaffold, the scaffold further comprises a distal attachment zone, a proximal attachment zone and an unattached zone.

59. The vascular occlusion device of any one of claims 53-56 wherein the multiple layer scaffold covering further comprises a proximal attachment zone, a distal attachment zone and an unattached zone wherein a thickness of the multiple layer covering in the proximal attachment zone and the distal attachment zone is greater than the thickness of the multiple layer scaffold covering in the unattached zone.

60. The vascular occlusion device of claim 59 wherein the multiple layer scaffold covering on the scaffold structure has a thickness of 5-100 microns.

61. The vascular occlusion device of any one of claims 53-56 wherein scaffold structure has a cylindrical portion and a conical portion wherein the terminal ends of the conical portion are coupled to the inner shaft.

62. The vascular occlusion device of any one of claims 53-56 wherein the inner shaft further comprises one or more spiral cut sections to increase flexibility of the inner shaft.

63. The vascular occlusion device of claim 62 wherein the one or more spiral cut sections are positioned proximally or distally or both proximal and distal to an inner shaft coupler where the scaffold structure is attached to the inner shaft.

64. The vascular occlusion device of any one of claims 53-56 the scaffold structure further comprising two or more legs wherein each of the two or more legs terminates with a connection tab that is joined to a corresponding key feature on an inner shaft coupler.

65. The vascular occlusion device of any one of claims 53-56 wherein the single or the multiple layer scaffold covering includes one or more or a pattern of apertures that are shaped, sized or positioned relative to the scaffold structure to modify the amount of distal perfusion provided by the vascular occlusion device in use within the vasculature.

66. The vascular occlusion device of any one of claims 53-56 wherein the single or the multiple layer scaffold covering includes one or more regular or irregular geometric shapes arranged in a continuous or discontinuous pattern which is selected to adapt the distal perfusion flow profile of the vascular occlusion device in use within the vasculature.

67. The vascular occlusion device of any one of claims 1, 13, 28, 31, 35, 38 and 53 wherein when in a stowed configuration within the outer shaft the overall diameter is between 0.100 inches and 0.104 inches and when in a deployed configuration the covered scaffold has an outer diameter from 19 to 35 mm.

68. The vascular occlusion device of any one of claims 1, 13, 28, 31, 35, 38 and 53 wherein the covered scaffold has an occlusive length of 40 mm to 100 mm measured from a distal end of the scaffold to a scaffold transition zone.

69. An introducer with an occlusion and perfusion device according to any one of claims 1, 13, 28, 31, 35, 38 and 53 adapted for or used to perform an intravascular procedure in a radial artery, an ulnar artery, a coronary artery, a posterior tibial artery, a fibular artery, an anterior tibial artery, a popliteal artery, a vein, a femoral artery or a portion of an aorta.

70. An introducer with an occlusion and perfusion device according to any one of claims 1, 13, 28, 31, 35, 38 and 53 adapted for or used to perform an intravascular procedure wherein the intravascular device is at least one of a diagnostic instrument, an angiography catheter, a balloon catheter, a drug-eluting balloon catheter, a bare metal stent, a drug-eluting stent, a drug-eluting biodegradable stent, an intravascular ultrasound testing instrument, a rotablator, a thrombus suction catheter, a drug administration catheter, a prosthesis for a portion of the vasculature, a prosthesis for a portion of an organ, a prosthesis for a portion of a heart, a prosthetic heart valve, or a device described in Appendix A or used in TMVR, TTVR, TAVR or other transcatheter coronary repair or replacement component, device, system of procedure.

71. An introducer with an occlusion and perfusion device according to any one of claims 1, 13, 28, 31, 35, 38 and 53 the introducer further comprising an expansion capability along all or a portion of the length of the introducer wherein the expansion capability is provided by one or more of a selection of flexible biocompatible polymers alone or in any combination with a braided portion.

72. The method of any one of claims 28, 31, 35 and 38 wherein a portion of an unattached zone of a multiple layer scaffold covering distends in response to blood flow along a lumen of the scaffold of the vascular occlusion device to occlude an opening of any of a hepatic artery, a gastric artery, a celiac trunk, a splenic artery, an adrenal artery, a renal artery, a superior mesenteric artery, an ileocolic artery, a gonadal artery and an inferior mesenteric artery.

73. The device of claim 13 wherein the length of the occlusion device pocket is sufficient to hold an occlusion device having a therapeutic length 1, a therapeutic length 2 or a therapeutic length 3.
